(19) United States
(12) Patent Application Publication
(10) Pub. No.: US 2022/0028529 A1
(43) Pub. Date: Jan. 27, 2022

(54) METHODS AND SYSTEMS FOR TREATING GASTROINTESTINAL AND INFLAMMATORY HEALTH CONDITIONS USING PRESCRIPTION DIGITAL THERAPEUTICS

(71) Applicant: Mahana Therapeutics, Inc., San Francisco, CA (US)

(72) Inventors: Robert Bradley Paull, Tiburon, CA (US); Gaurav Kumar, San Francisco, CA (US); Christopher Michael Browning, Concord, CA (US); Megan Leigh Oser, San Francisco, CA (US); Ann Harriet Montgomery, San Anselmo, CA (US); Jennifer Lily Shim, Oakland, CA (US); Simon Levy, Denver, CO (US)

(73) Assignee: Mahana Therapeutics, Inc., San Francisco, CA (US)

(21) Appl. No.: 17/497,501

(22) Filed: Oct. 8, 2021

Related U.S. Application Data
(63) Continuation-in-part of application No. 16/906,085, filed on Jun. 19, 2020.
(60) Provisional application No. 63/104,322, filed on Oct. 22, 2020.
(60) Provisional application No. 62/867,275, filed on Jun. 27, 2019.

(51) Int. Cl. G16H 20/70; G16H 50/20; A61B 5/00; G09B 19/00

(52) U.S. Cl. CPC G16H 20/70; G16H 50/20; G09B 19/00; A61B 5/4836; A61B 5/7282; A61B 5/4255

(57) ABSTRACT

A prescription digital therapeutic (PDT) system is provided to patients/users suffering from gastrointestinal (GI) and/or inflammatory health conditions, wherein the PDT system allows guided behavioral therapy and skills training to be administered in a convenient and flexible, yet structured fashion, via a system associated with an application such as a mobile application. Guided behavioral therapy technologies may be based at least in part on cognitive behavioral therapy (CBT) techniques to allow for development of a skillset for treating gastrointestinal (GI) and/or inflammatory diseases, disorders and/or conditions, and for managing stress and/or other psychological symptoms associated with such diseases, disorders and/or conditions. Patient interactions with the PDT system are monitored to control progression through the system content and to continually refine one or more personalized intervention regimens associated with the guided behavioral therapy.

### RELATED APPLICATIONS

This application is a continuation-in-part of Paull et al., U.S. patent application Ser. No. 16/906,085, filed on Jun. 19, 2020, entitled “ADAPTIVE INTERVENTIONS FOR GASTROINTESTINAL HEALTH CONDITIONS,” which claims the benefit of Paull et al., U.S. Provisional Application No. 62/867,275 filed on Jun. 27, 2019, entitled “ADAPTIVE INTERVENTIONS FOR GASTROINTESTINAL HEALTH CONDITIONS.” This application also claims the benefit of Paull et al., U.S. Provisional Application No. 63/104,322, filed on Oct. 22, 2020, entitled “SYSTEMS AND METHODS FOR GUIDED ADMINISTRATION OF BEHAVIORAL THERAPY.” Each of the above applications is hereby incorporated by reference in their entirety as if they were fully set forth herein.

This application is related to Levy, U.S. patent application Ser. No. 16/717,291, filed on Dec. 17, 2019, entitled “METHOD AND SYSTEM FOR REMOTELY MONITORING THE PSYCHOLOGICAL STATE OF AN APPLICATION USER BASED ON HISTORICAL USER INTERACTION DATA.” This application is related to Levy, U.S. patent application Ser. No. 16/888,061, filed May 29, 2020, entitled “METHOD AND SYSTEM FOR REMOTELY IDENTIFYING AND MONITORING ANOMALIES IN THE PHYSICAL AND/OR PSYCHOLOGICAL STATE OF AN APPLICATION USER USING BASELINE PHYSICAL ACTIVITY DATA ASSOCIATED WITH THE USER.” This application is related to Levy, U.S. patent application Ser. No. 17/216,993, filed Mar. 30, 2021, entitled “METHOD AND SYSTEM FOR DYNAMICALLY GENERATING PROFILE-SPECIFIC THERAPEUTIC PROTOCOLS USING MACHINE LEARNING MODELS.” This application is related to Levy, U.S. patent application Ser. No. 17/217,052, filed on Mar. 30, 2021, entitled “METHOD AND SYSTEM FOR DYNAMICALLY GENERATING PROFILE-SPECIFIC THERAPEUTIC IMAGERY USING MACHINE LEARNING MODELS.” Each of the above applications is hereby incorporated by reference in their entirety as if they were fully set forth herein.

### BACKGROUND

In recent years, digital applications have come to play an increasingly large role in the daily lives of billions of people all over the world. Currently, a vast number of applications are readily available to users over a wide variety of technologies. These applications range greatly in type and purpose, providing users with information and services such as productivity tools, educational materials, and entertainment options. As technology advances, these applications are becoming increasingly sophisticated in terms of the content and experiences they are able to provide to users. For example, in addition to providing users with information and other types of static content, most modern applications are also able to provide users with a variety of interactive features, thereby allowing a user to select specific and/or customized content based on user input, user interactions, and user behavior. In this way, the benefits that applications provide to a user can be customized to meet the needs or desires of specific individuals.

Due to the increased use of these digital applications in the daily lives of users, many such applications are now being used to supplement or replace traditional human to human, i.e., in person, interactions. Further, it has become increasingly clear that this trend will continue to grow in the years to come. However, while these types of interactive applications can provide many beneficial features to users, the underlying systems that enable these applications to provide beneficial features to users are growing more and more technically complex. As such, the technical complexities of modern application systems present a variety of challenges that need to be addressed in order for this interactive technology to achieve its fullest potential.

As a specific example, every day, millions of people are diagnosed with a wide variety of medical conditions, ranging greatly in type and severity. A patient who has been diagnosed with a medical condition often experiences many hardships as a result of their diagnosis. In addition to physical effects, such as pain, discomfort, or loss of mobility that may accompany the diagnosis, the hardships faced by patients often further include financial difficulties resulting from lost work, medical bills and the cost of treatments. Further still, a patient's diagnosis often negatively impacts their social interactions and overall emotional well-being. The result is that many patients experience significant psychological distress as a result of their diagnosis, and often do not receive adequate support or treatment to alleviate this distress.

Often, when a patient is diagnosed with one or more medical conditions, the patient may be referred to additional health professionals for further care and treatment. For example, a patient may be referred to a psychologist, psychiatrist, counselor, or other mental health professional. A patient may also be directed to one or more support groups to assist with any psychological distress that the patient may be experiencing. While these traditional face-to-face options may be greatly beneficial to a patient, often times they do not provide enough psychological support. For example, when a patient is alone, at home, or not otherwise engaged directly with their mental health professional or support group, they may experience a significant degree of one or more negative emotional states, such as fear, anxiety, panic, and depression. Additionally, left unidentified and untreated, these negative emotional states often exacerbate the physical symptoms associated with a patient's diagnosis, which in turn can lead to greater psychological distress.

As specific examples, gastrointestinal (GI) health conditions and inflammatory health conditions have significant impact on the quality of life of patients worldwide. An estimated 60-70 million people in the U.S. alone have diagnosed GI health conditions, with millions more undiagnosed individuals experiencing symptoms but failing to receive treatment. In relation to treatment and therapy for GI and/or inflammatory health conditions, current approaches typically focus on reducing or eliminating physiological symptoms, by implementation of medication regimens, supplement regimens, diet changes, and/or lifestyle changes. However, GI and/or inflammatory health conditions have other adverse effects on lives of patients due to the nature of symptoms, and current approaches to treatment fail to address such adverse effects. Furthermore, current methods of improving patient states associated with GI and/or inflammatory health conditions are limited in relation to: educating patients regarding standard and non-standard treatment options; detecting, in real or near-real time, states of symptom severity in non-invasive manners; and delivering therapy in a customized and adaptive manner.

One type of therapy that has been shown to improve patient physiological states is behavioral therapy. Behavioral therapy is also a valuable tool for addressing a variety of mental health issues, including anxiety and depression, and improving an individual's ability to manage and respond effectively to external stimuli, such as stress. Typically, behavioral therapy is administered via a mental health professional, such as a therapist, for example through regular sessions between an individual and their therapist. Such interactions, however, can be time consuming, inconvenient, and costly, thereby limiting accessibility of behavioral therapy treatment.

Because current mechanisms for enabling mental health professionals to remotely provide behavioral therapy treatment to patients suffering from GI and/or inflammatory health conditions outside of a medical office or support group setting are limited, the shortcomings associated with traditional psychological support and treatment options presents a technical problem, which requires a technical solution. As digital applications and systems continue to replace human interactions, this problem becomes even more pronounced, due to the fact that people are increasingly relying on digital applications to provide them with support and assistance in a wide variety of aspects of their daily lives. As such, the failure of traditional solutions to address the technical system complexities involved with providing remote care to patients suffering from GI and/or inflammatory health conditions has the potential to lead to significant consequences for a large number of people.

Thus, there is a need in the fields of GI and inflammatory health care and digital therapeutics to provide a new and useful system and method for detecting patient states and providing adaptive interventions for improving patient states, including systems and methods for treating gastrointestinal and inflammatory health conditions using prescription digital therapeutics in order to ensure that patients receive adequate care, support, and treatment.

### SUMMARY

Embodiments of the present disclosure provide a technical solution to the technical problem of effectively, efficiently, and remotely treating gastrointestinal and inflammatory health conditions using prescription digital therapeutics in order to ensure that patients receive adequate care, support, and treatment. The inventions covered by the system and method disclosed herein can confer several benefits over conventional systems and methods, and such inventions are further implemented into many practical applications related to improvement of technologies utilized for patient healthcare.

The systems and methods disclosed herein allow behavioral therapy to be remotely administered to patients suffering from gastrointestinal (GI) and/or inflammatory health conditions in a convenient and flexible, yet structured fashion, via a prescription digital therapeutics (PDT) system. In some embodiments, the invention(s) disclosed herein can employ non-traditional systems and methods for providing services such as interventions to patients exhibiting symptoms associated with one or more GI and/or inflammatory health conditions. In some embodiments, the invention(s) can deliver psychological-based interventions to patients, such as, but not limited to, cognitive behavioral therapy (CBT)-based interventions, as well as other types of interventions, which are described in more detail below, by way of a platform having components implemented in a mobile device environment and/or other computer or internet-based architecture. Thus, in various embodiments, the invention(s) use components of the platform to process large amounts of user data, remotely deliver personalized interventions, and remotely monitor user interactions with such interventions in near real-time in a manner that cannot be practically implemented by the human mind.

In some embodiments, prescription digital therapeutics (PDT) technologies (e.g., systems and methods) disclosed herein may be used to administer behavioral therapy treatments in a controlled fashion, as treatment for one or more conditions described herein. In some embodiments, provided technologies address physiological conditions (e.g., conditions with one or more physical symptoms, features, or manifestations) that may be affected by a subject's mental health state, for example, presence of a mental health condition such as, but not limited to anxiety, depression, and/or stress. As discussed herein, mental health conditions and/or stress levels may be triggered and/or worsened by symptoms of a particular associated physiological condition, and/or may trigger and/or worsen the symptoms of the same particular physiological conditions, for example, in a feedback-like manner, which is often referred to as a ‘vicious cycle.’

Accordingly, in some embodiments, the PDT technologies disclosed herein can be used to administer guided behavioral therapies to treat individuals suffering from certain particular physiological conditions. As described herein, GI and/or inflammatory health conditions are particularly relevant, thus, in certain embodiments, guided behavioral therapy approaches disclosed herein may be administered to individuals suffering from one or more GI and/or inflammatory health conditions. In this manner, approaches described herein may be used to ameliorate symptoms associated with a variety of GI and/or inflammatory health conditions.

In one embodiment, a patient is provided with a user interface to a prescription digital therapeutics (PDT) system wherein the PDT system remotely administers guided behavioral therapy through an intervention regimen defining a plurality of interactive therapy modules to be administered to the patient.

In one embodiment, a pre-assessment of a patient exhibiting one or more GI and/or inflammatory health condition symptoms is performed by the PDT system to generate patient profile and pre-assessment data.

In one embodiment, the patient profile and pre-assessment data is processed by the PDT system to generate patient condition data, wherein the patient condition data includes an identification of the patient's condition, condition sub-type, and/or condition severity.

In one embodiment, the patient profile and pre-assessment data and the patient condition data are processed by the PDT system to generate a personalized intervention regimen for the patient, wherein the personalized intervention regimen defines one or more interactive therapy modules to be administered to the patient in a manner tailored to the patient's needs.

In one embodiment, the one or more interactive therapy modules are administered, through the user interface of the PDT system, to the patient according to the personalized intervention regimen generated for the patient.

In one embodiment, the patient's interactions with the content of the interactive therapy modules are monitored remotely in near real-time to generate patient interaction data.

In one embodiment, at least partly based on the patient interaction data, patient progression through the interactive therapy modules is dynamically and remotely controlled.

In one embodiment, at least partly based on the patient interaction data, the patient's personalized intervention regimen is dynamically updated.

In some embodiments, the invention(s) can also provide interventions that are tailored to individual users/patients suffering from a variety of symptoms, such as, but not limited to, symptoms associated with digestion, defecation, various stooling symptoms, pain, social/interpersonal effects, emotional effects, cognitive effects, and behavioral effects, in a customized manner, with implementation of real-time or near real-time assessments of data from multiple sources, including, but not limited to, electronic health record sources, self-report sources, and sensor sources.

In some embodiments, the invention(s) can also be used for acquisition of user/patient data from multiple data sources, including, but not limited to, health data, biometric data, user demographic data, and user behavior data. In this regard, the invention(s) can also be used for generation of training datasets, whereby the training datasets can be used for training machine learning models (e.g., neural networks, etc.) that take input data pertaining to users/patients and produce outputs that can be used to guide customization of interventions.

In some embodiments, the invention(s) can also be used to provide automated delivery of health-promoting or improving interventions, automated tracking/monitoring of user interactions with such interventions, automated communications with users (e.g., through transmission of notifications), and/or automated delivery of modified interventions to users, through a mobile device application platform and/or other platform (e.g., web platform).

In some embodiments, such interventions can also be delivered as digital therapeutics, alone as a monotherapy or in combination with other therapeutics, such as medications and/or medical devices, through technical systems intended to diagnose and/or treat and/or improve symptoms or health-related quality of life, in collaboration with healthcare providers, health insurers, and/or other entities in the healthcare system. The invention(s) can also employ non-traditional systems and methods for delivering prescription digital therapeutics (PDT) for improving patient health (e.g., in relation to disease management), whereby digital therapeutics are prescribed through healthcare providers (e.g., with associated billing codes).

Additionally or alternatively, in some embodiments, the invention(s) can include systems and methods for improving patient states (e.g., in the context of health, symptoms, disease progression, quality of life, and other contexts). Additionally or alternatively, in some embodiments, the system and/or method can confer any other suitable benefit.

In some embodiments, the present disclosure provides methods for remotely administering behavioral therapy to a user/patient via a controlled progression of interactive therapy modules, through a graphical user interface (GUI) of a prescription digital therapeutics (PDT) system. In some embodiments, technologies described herein allow an individual user/patient to access and take part in a series of guided lessons that provide training in various behavioral skills. In one embodiment, these guided lessons may be presented as a sequence of interactive lesson modules that provide training and practice via a graphical user interface (GUI) of a PDT system. In some embodiments, approaches described herein include control features that manage progression of a user through lesson sequences.

In some embodiments, approaches described herein provide structured behavioral therapy that is targeted at managing triggers and/or symptoms associated with specific physical conditions. Accordingly, in some embodiments, a behavioral therapy toolkit as provided by the systems and methods described herein can be tailored for a particular physical condition.

In some embodiments, the present disclosure provides methods for providing for interactive creation of a user personal model via a graphical user interface (GUI) of a PDT system, allowing a user to identify cycles of behaviors, thoughts, emotions, and stressors that influence the frequency and/or severity of symptoms associated with a particular physical condition from which the user is suffering,

Thus, among other things, technologies described herein can increase access to and/or facilitate effective administration of behavioral therapy, and moreover can achieve effective impact on physiological conditions through guided behavioral therapy. In some embodiments, the present disclosure provides improvements to technologies and methods for administering cognitive behavioral therapy (CBT). In some embodiments, the present disclosure provides improvements to technologies for administering a wide variety of therapy modalities, individually, or in combination with other modalities.

Consequently, embodiments of the present disclosure provide a technical solution to the technical problem of effectively, efficiently, and remotely treating gastrointestinal and inflammatory health conditions using prescription digital therapeutics in order to ensure that patients receive adequate care, support, and treatment.

### BRIEF DESCRIPTION OF THE DRAWINGS

FIG. 1A depicts a schematic of a system for treating gastrointestinal and inflammatory health conditions using prescription digital therapeutics, according to one or more embodiments.

FIG. 1B depicts a block diagram of a production environment for treating gastrointestinal and inflammatory health conditions using prescription digital therapeutics, according to one or more embodiments.

FIG. 2A depicts a flowchart of a method for treating gastrointestinal and inflammatory health conditions using prescription digital therapeutics, according to one or more embodiments.

FIG. 2B depicts a flowchart of a method for providing adaptive interventions for gastrointestinal and inflammatory health conditions, according to one or more embodiments.

FIG. 2C depicts a flowchart of a method for providing adaptive interventions for gastrointestinal and inflammatory health conditions, according to one or more embodiments.

FIG. 3A depicts a schematic of architecture implemented for delivery of intervention regimen components and/or modules, according to one or more embodiments.

FIG. 3B depicts examples of individual sections that may make up an introduction and education module of an intervention regimen, according to one or more embodiments.

FIG. 3C depicts examples of individual sections that may make up a pain management module of an intervention regimen, according to one or more embodiments.

FIG. 4 depicts an example of formation of a personal disease model, according to one or more embodiments.

FIG. 5A depicts a flowchart of a process for determining severity of a gastrointestinal and/or inflammatory health condition, according to one or more embodiments.

FIG. 5B depicts examples of a process for determining severity of a gastrointestinal and/or inflammatory health condition, according to one or more embodiments.

FIG. 6 depicts a flowchart of a pre-assessment and onboarding process of a method for providing adaptive interventions, according to one or more embodiments.

FIG. 7 depicts examples of system aspects of a program for personalized health condition monitoring and improvement, according to one or more embodiments.

FIG. 8A, FIG. 8B, FIG. 8C, FIG. 8D, and FIG. 8E depict example schematics of conditional branching architecture implemented for delivery of intervention regimen components, according to one or more embodiments.

FIG. 9A, FIG. 9B, FIG. 9C, and FIG. 9D are screenshots of several portions of an exemplary GUI for a system for treating gastrointestinal and/or inflammatory health conditions using digital therapeutics, according to one or more embodiments.

FIG. 10A, FIG. 10B, FIG. 10C, and FIG. 10D are screenshots of example user interactions with an initial lesson module for content tailored for a patient with IBS, according to one or more embodiments.

FIG. 11A and FIG. 11B are screenshots showing gate features of an exemplary GUI for a system for treating gastrointestinal and/or inflammatory health conditions using digital therapeutics, in accordance with one or more embodiments.

FIG. 12A, FIG. 12B, FIG. 12C, and FIG. 12D are screenshots of an exemplary GUI for a symptom diary lesson module, according to one or more embodiments.

FIG. 13A, FIG. 13B, FIG. 13C, and FIG. 13D are screenshots of example user interactions with a symptom diary practice module, in accordance with one or more embodiments.

FIG. 14A, FIG. 14B, FIG. 14C, and FIG. 14D are screenshots of an exemplary GUI for introducing a personal model lesson module, in accordance with one or more embodiments.

FIG. 15A, FIG. 15B, FIG. 15C, and FIG. 15D are screenshots of an exemplary GUI for a personal model lesson module, according to one or more embodiments.

FIG. 16A, FIG. 16B, and FIG. 16C are screenshots of an exemplary personal model graphical representation, according to one or more embodiments.

FIG. 17A, FIG. 17B, FIG. 17C, and FIG. 17D are screenshots of an exemplary GUI for a reflections section of a personal model lesson module, according to one or more embodiments.

FIG. 18A, FIG. 18B, FIG. 18C, and FIG. 18D are screenshots of an exemplary GUI for a symptom management lesson module, according to one or more embodiments.

FIG. 19A, FIG. 19B, FIG. 19C, and FIG. 19D are screenshots of an exemplary GUI for an unhelpful thought pattern lesson module, according to one or more embodiments.

Common reference numerals are used throughout the figures and the detailed description to indicate like elements. One skilled in the art will readily recognize that the above figures are merely illustrative examples and that other architectures, modes of operation, orders of operation, and elements/functions can be provided and implemented without departing from the characteristics and features of the invention, as set forth in the claims.

### DETAILED DESCRIPTION

Embodiments will now be discussed with reference to the accompanying figures, which depict one or more exemplary embodiments. Embodiments may be implemented in many different forms and should not be construed as limited to the embodiments set forth herein, shown in the figures, or described below. Rather, these exemplary embodiments are provided to allow a complete disclosure that conveys the principles of the invention, as set forth in the claims, to those of skill in the art.

The systems and methods disclosed herein allow behavioral therapy to be administered to patients suffering from gastrointestinal (GI) and/or inflammatory health conditions in a convenient and flexible, yet structured fashion, via a prescription digital therapeutics (PDT) system.

In various embodiments, behavioral therapy may include therapies such as, but not limited to, psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy.

In some embodiments, such guided behavioral therapy technologies are based at least in part on cognitive behavioral therapy (CBT), and provide structured modules and/or lessons via a graphical user interface (GUI) of a prescription digital therapeutics (PDT) system, for example to allow patients to develop a skillset for treating a physiological disease, disorder and/or condition, and for managing stress and/or other psychological symptoms associated with such disease, disorder and/or condition. In some embodiments, a relevant disease, disorder or condition may be or comprise an inflammatory health condition and/or a GI health condition.

In some embodiments, approaches described herein provide patients with structured behavioral therapy that is targeted at managing triggers and/or symptoms associated with specific physical and/or physiological conditions. In particular, many physiological conditions, such as GI and/or inflammatory health conditions, can be triggered and/or exacerbated by stress and mental health disorders such as anxiety and/or depression. Moreover, at the same time, symptoms and effects of these physiological conditions may trigger and/or worsen related mental health conditions and can be a source of significant stress for many patients. This feedback loop can create a vicious cycle, in which symptoms of a particular physiological condition trigger and/or worsen mental health states, causing issues such as anxiety and/or depression, which, in turn, trigger and/or worsen physical/physiological symptoms. Feedback loops of this kind are particularly relevant to GI and inflammatory health conditions. As one illustrative example, symptoms associated with irritable bowel syndrome (IBS) are often triggered by stress, and mental health conditions such as depression and/or anxiety can worsen such symptoms. At the same time, physical pain, as well as the many complexities of managing IBS (e.g., avoiding certain foods, planning to be in proximity to a bathroom, etc.), are themselves significant stressors and may contribute to depression and/or anxiety.

Accordingly, in some embodiments, a behavioral therapy toolkit as provided by technologies (e.g., systems and methods) disclosed herein can be tailored for a particular physical/physiological condition. For example, particular behavioral therapy lesson modules can be designed to facilitate tracking of specific symptoms, tracking and elucidating specific stressors, and/or providing targeted exercises (e.g., breathing, progressive relaxation, etc.), which are well suited for managing specific triggers and symptoms of the particular physiological condition. For example, as described herein, behavioral therapy lesson modules targeted at a GI health condition, such as IBS, may include specially designed symptom diaries, meal trackers, personal model interfaces and/or other types of therapy modules, which allow a user to discover and reflect upon combinations of stressors, triggers, thoughts, and symptoms that contribute to their own, personal, vicious cycle.

Accordingly, by providing a toolkit for managing mental health issues that are linked with GI and/or inflammatory health conditions, the technologies, methods, and systems disclosed herein provide a valuable complement to physician visits and recommendations (e.g., with regard to lifestyle changes) and standard of care therapies (e.g., administration of medication(s)). Furthermore, technologies disclosed herein can increase access to and/or facilitate effective administration of behavioral therapy to achieve effective impact on physiological conditions such as, but not limited to, GI and/or inflammatory health conditions, through guided behavioral therapy.

### Term Definitions

As used herein, the term “patient,” and/or “subject,” may include an individual who is suffering from a relevant disease, disorder or condition. In some embodiments, a patient/subject is an individual who is susceptible to a disease, disorder, or condition. In some embodiments, a patient/subject displays one or more symptoms or characteristics of a disease, disorder or condition. In some embodiments, a patient/subject does not display any symptom or characteristic of a disease, disorder, or condition. In some embodiments, a patient/subject is someone with one or more features characteristic of susceptibility to or risk of a disease, disorder, or condition. In some embodiments, a patient/subject is an individual to whom diagnosis and/or therapy is and/or has been administered. In some embodiments, a patient/subject is an individual who has been diagnosed with one or more diseases, disorders, and/or conditions and is the recipient of one or more therapies in a clinical or non-clinical setting. In some embodiments, a patient/subject is an individual who has not been diagnosed with a health condition, but is a recipient of one or more therapies in a clinical or non-clinical setting.

As used herein, the term “therapeutics system,” “prescription digital therapeutics (PDT)” and/or “prescription digital therapeutics (PDT) system,” may include a system utilized for remotely administering a therapy to a patient, wherein the PDT system is required to be approved by a government agency before it can be marketed for administration to humans. Typically, a PDT system requires FDA approval and rigorous clinical evidence to substantiate intended use and impact on disease state. Further, a PDT system is typically a system for which a medical prescription is required for administration to patients.

As used herein, the term “user” may include a patient/subject who utilizes a therapeutics system or a prescription digital therapeutics (PDT) system.

As used herein, an individual who is “suffering from” a disease, disorder, and/or condition displays one or more symptoms of a disease, disorder, and/or condition and/or has been diagnosed with the disease, disorder, or condition.

As used herein, the term “therapy,” “behavioral therapy,” and/or “guided behavioral therapy” may include psychological techniques, methodologies, and/or modalities intended or demonstrated to achieve impact on and/or alteration of one or more behaviors of a patient/subject. Examples of therapies may include, but are not limited to, psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy.

As used herein, the term “mind-body intervention,” may include one or more therapeutic practices that employ a variety of techniques designed to facilitate the mind's capacity to affect bodily function and systems. Examples of mind-body interventions may include, but are not limited to, relaxation, imagery, biofeedback, meditation, hypnosis, tai, chi, and yoga.

As used herein, the phrase “administration” may include providing, delivering, and/or applying a therapy to a patient. A therapy may be administered to a patient directly by a health practitioner. A therapy may be administered to a patient remotely, for example, over the internet or through a computer system, without the direct involvement of a health practitioner. For example, the therapy may be self-administered by the patient. A therapy may also be administered to a patient remotely with partial involvement of a health practitioner. For example, the therapy to be administered may be selected by a health practitioner, but the therapy may then be self-administered by the patient, utilizing a computer system, or the therapy may be administered to the patient by a computer system, but a health practitioner may monitor the patient's response data. Those of ordinary skill in the art, reading the present disclosure, will appreciate, for example, that a variety of routes are available for administration of compositions; for example, some compositions may be administered by one or more routes such as ocular, oral, parenteral, topical, etc. Furthermore, the present disclosure, in some embodiments, describes administration of behavioral therapy, for example via interaction with a counselor (e.g., a therapist) and/or with a device or computing system as described herein. In some embodiments, administration may involve dosing, application, or interaction that is intermittent (e.g., a plurality of doses separated in time) and/or periodic (e.g., individual doses separated by a common period of time) dosing. In some embodiments, administration may involve continuous dosing (e.g., perfusion), application or interaction for at least a selected period of time.

As used herein, the term “treat,” “treatment,” or “treating” may include administration of therapy that has been established to partially or completely alleviate, ameliorate, relieve, inhibit, prevent, delay onset of, reduce severity of, and/or reduce incidence of one or more symptoms or features of a disease, disorder, and/or condition (e.g., when administered to a relevant population). In some embodiments, treatment may be administered to a patient who is not exhibiting (and/or has not exhibited) one or more signs of a relevant disease, disorder, and/or condition. In some embodiments, treatment may be administered to a patient who exhibits only early signs of the disease, disorder, and/or condition, for example for the purpose of decreasing risk of developing one or more features of pathology associated with the disease, disorder, and/or condition. In some embodiments, a treatment is termed “therapeutic” when administered to a patient who is displaying or has displayed one or more features, symptoms, or other characteristics of a relevant disease, disorder and/or condition. In some embodiments, a treatment is termed “prophylactic” when administered to a patient who has not displayed features, symptoms, or other characteristics of a relevant disease, disorder and/or condition.

As used herein, the term “protocol” or “therapeutic protocol” may include procedures and/or systems of rules for administration of a therapy. A therapeutic protocol defines the rules, syntax, semantics, and synchronization of communications with a patient. For example, a therapy may include a series of modules, lessons, questionnaires, and exercises, and a related protocol may dictate the order, speed, and/or frequency in which various modules, lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. A therapy may be administered to a patient according to any number of protocols or any number of combinations of protocols.

As used herein, the term “therapeutic regimen” or “intervention regimen” may include a therapy for administration to a patient as part of a therapeutic treatment, wherein the therapy is administered to the patient according to a specific set of therapeutic protocols. For example, a therapeutic/intervention regimen may include a specific set of modules, lessons, questionnaires, exercises, and other content, which may be administered to a patient in a particular order, at a particular frequency, utilizing a particular layout, etc. In some embodiments, a therapeutic/intervention regimen may be correlated with a desired or beneficial therapeutic outcome. In some embodiments, a therapeutic/intervention regimen may be personalized or tailored to meet the needs of a specific patient.

As used herein, the term “therapeutic agent” may include any agent that elicits a desired effect when administered to an organism, e.g., in a pharmaceutical composition, via a prescription digital therapeutics (PDT) system, and/or according to a therapeutic regimen as described herein. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting conditions, etc. In some embodiments, a therapeutic agent can be used to alleviate, ameliorate, relieve, inhibit, prevent, delay onset of, reduce severity of, and/or reduce incidence of one or more symptoms or features of a disease, disorder, and/or condition.

As used herein, the term “amelioration” may include prevention (e.g., delay), reduction (e.g., in frequency and/or intensity), improvement, and/or palliation of a state, or one or more features thereof, experienced by a patient. Amelioration may include, but does not require, complete recovery or complete prevention of a disease, disorder or condition (e.g., radiation injury).

As used herein, the terms “improve,” “increase,” “inhibit,” “reduce,” or grammatical equivalents thereof, may include values that are relative to a baseline or other reference measurement. In some embodiments, an appropriate reference measurement may be or may comprise a measurement in a particular system (e.g., in a single individual) under otherwise comparable conditions absent (e.g., prior to and/or after addition of) a particular agent or treatment, or in presence of an appropriate comparable reference agent. In some embodiments, an appropriate reference measurement may be or may comprise a measurement in a comparable system known or expected to respond in a particular way, for example in presence of the relevant agent or treatment.

As used herein, the terms “prevent” and/or “prevention,” when used in connection with the occurrence of a disease, disorder, and/or condition, may include reducing a risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

As used herein, the term “patient illness narrative,” “personal illness narrative,” and/or “physical illness narrative” may include a narrative expressed by a patient regarding the patient's personal experiences with a disease, disorder, and/or condition. An illness narrative is typically a narrative solicited from a patient, which enables a healthcare practitioner to build a more complete picture of the patient's past and present health state in the context of the patient's life, while providing the patient with an opportunity for self-reflection and validation.

As used herein, the term “personal model” and/or “personal disease model” may include a construction built based on patient input, which enables the patient to identify stressors, counter-productive behaviors, unhelpful thoughts, and negative emotions as associated with the patient's disease, disorder, and/or condition. In some embodiments, a personal model is constructed as a graphical representation, which comprises text corresponding to patient-selected counter-productive behavior(s), unhelpful thought(s), and negative emotion(s), superimposed on a flow diagram illustrating links between the patient's behaviors, thoughts, and emotions. In some embodiments, a personal model graphical representation comprises text corresponding to causes and/or stressors of symptoms. A personal model may be utilized to help a patient identify links between their behaviors, thoughts, and emotions, and to help a patient consider possible changes in their behavior that could be implemented to address their symptoms.

As used herein, the term “ecological momentary assessment” may include repeatedly sampling a subject's current behaviors and experiences in real time, in the subject's natural environment, with the aim of minimizing recall bias and allowing study of microprocesses that influence behavior in real-world contexts.

As used herein, the term “machine learning module” may include a computer implemented process that implements one or more particular machine learning algorithms, such as supervised, unsupervised, and semi-supervised systems, an artificial neural network (ANN), random forest, decision trees, support vector machines, and the like, in order to determine, for a given input, one or more output values.

### System

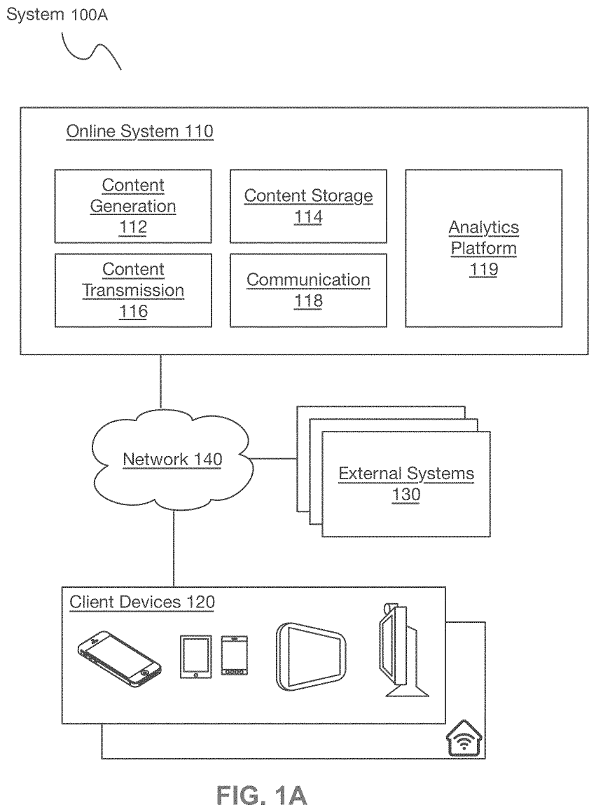

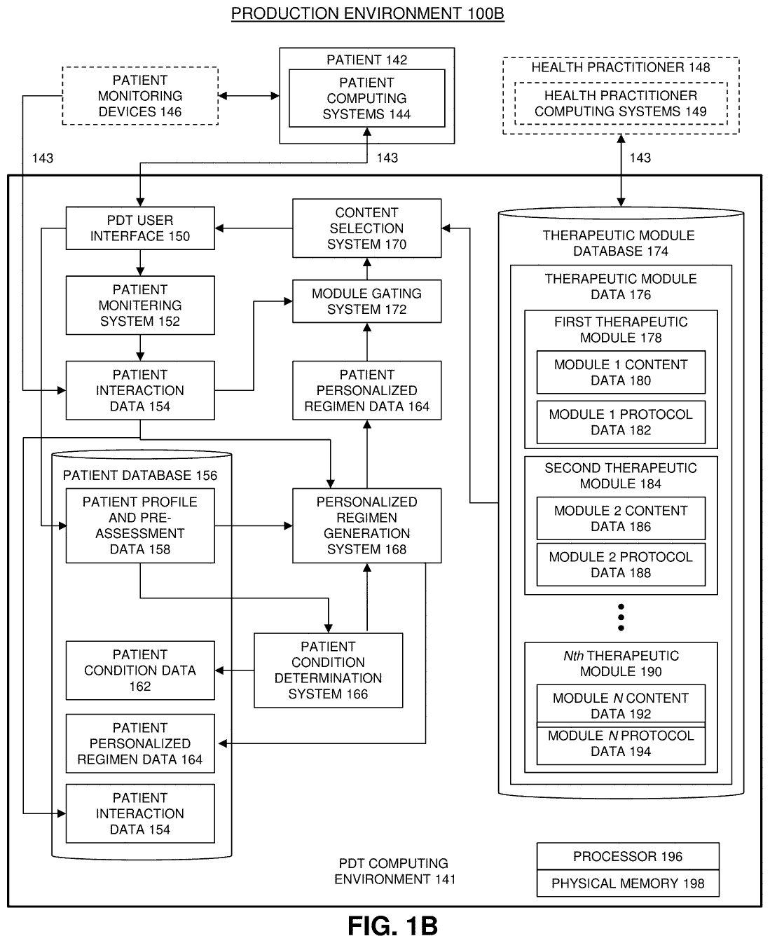

FIG. 1A depicts a schematic of a system 100A for treating gastrointestinal and inflammatory health conditions using prescription digital therapeutics, according to one or more embodiments.

As shown in FIG. 1A, in one embodiment, system 100A includes: an online system 110 for digital content associated with the adaptive interventions, one or more client devices including client device 120 for delivering the behavioral therapy and skills training to one or more users, one or more external systems including external system 130, and a network 140 for data transmission between the online system 110, the client device(s) 120, and the external system(s) 130. In one embodiment, the system 100A includes functionality for educating patients (e.g., patients, users of the platform, etc.) regarding treatment and therapy options in the context of improving symptoms associated with GI and/or inflammatory health conditions; detecting, in real or near-real time, states of GI and/or inflammatory health condition symptom severity in non-invasive manners; and delivering therapeutic interventions in a customized, and adaptive manner to one or more users/patients exhibiting GI and/or inflammatory health condition symptoms.

In some embodiments, the system 100A can provide tailored cognitive behavioral therapies (CBTs) and/or other therapeutic modalities, such as psychotherapy, acceptance commitment therapy (ACT), dialectical behavioral therapy (DBT), exposure therapy, mindfulness-based cognitive therapy (MCBT), hypnotherapy, experiential therapy, and psychodynamic therapy for patients in an adaptive and customizable manner. In some embodiments, components of the above-listed therapeutic modalities may be combined to tailor a therapeutic intervention regimen to the needs of a particular patient. Furthermore, while GI and/or inflammatory health conditions are indicated herein, variations of the system 100A can be adapted for generation and provision of interventions for systems associated with other health conditions.

In one embodiment, the online system 110 functions to generate, store, and transmit digital content associated with the behavioral therapy and related adaptive interventions, according to algorithms that allow the online system 110 to administer (or guide administration) of interventions to patients in a timely and customized manner. The online system 110 thus procures digital content associated with one or more therapeutic interventions and allows users/patients of the system 100A to access the digital content in an active or passive manner, in order to improve the patient(s)′ ability to manage GI and/or inflammatory health condition symptoms. In some embodiments, the online system 110 can include content generation components 112, content storage components 114, content transmission components 116, communication elements 118, and/or analytic platform 119 elements, implemented in computer architecture. The online system 110 can additionally or alternatively include any other suitable subsystems or components associated with administration of guided therapy, adaptive interventions, and/or monitoring of patient health condition states.

In relation to content generation components 112, in some embodiments, the online system 110 can include computing architecture configured for generation of interactive digital objects in computer-readable formats, where such interactive digital objects can be included in modules of therapeutic interventions provided to patients exhibiting one or more GI and/or inflammatory health condition symptoms. In various embodiments, the content generation components 112 can include architecture for generation of content in one or more of: visual formats (e.g., with image objects, video objects, etc.), audible formats, haptic formats, and any other suitable formats. Such content can be delivered through output devices of other components of the system 100A, such as display components (e.g., of a device, of an augmented reality device, of a virtual reality device, etc.), speaker components, haptic output device components, and/or any other suitable components.

In relation to content storage components 114, in some embodiments, the online system 110 can include architecture for storage and retrieval of computer-readable media associated with digital content and/or other objects. Data storage systems can be associated with any suitable format, and include components configured for cloud and/or non-based cloud computing. In particular embodiments, the information stored in the content storage components 114 can be organized according to specific data structures (e.g., with relational, columnar, correlation, or other suitable architecture). Stored content can be associated with various digital objects (e.g., graphical/textual/audio/visual/haptic objects associated with content, and/or rearrangement of objects within particular environments, as associated with therapeutics and/or communications between entities, as described in more detail below).

In relation to content transmission components 116, in some embodiments, the online system 110 can be configured to transmit content over wired and/or wireless interfaces, through network 140 (described in more detail below). As such, the content transmission components 116 of the online system 110 can include interfaces to the network 140, for content transmission to client devices 120 and/or external systems 130.

In relation to communication elements 118, in some embodiments, the online system 110 can include elements that enable communications between patients and other entities (e.g., care providers, coaches associated with health interventions, other patients, etc.) in text format, in audio format, and/or in any other suitable formats. In examples, the online system 110 can support messaging, calling, and/or any other suitable communication types using web or other computer-based communication subsystems.

In relation to analytics, in some embodiments, the online system 110 can include architecture for an analytics platform 119 for performing analytics in relation to generation of interventions (e.g., digital therapeutics as monotherapies, digital therapeutics as combinatorial therapies), evaluation of performance of interventions (e.g., in relation to performance, in relation to effectiveness, etc.), modification of interventions (e.g., in relation to content aspects, in relation to frequency aspects, etc.), provision of interventions (e.g., delivery method, etc.), generating and processing training data for refinement of models for intervention generation and provision, and other architecture for performing analytics.

In some embodiments, one or more portions of the online system 110 can include processing subsystem components comprising non-transitory media storing instructions for executing one or more method operations described below. The processing subsystem components can be distributed across the online system 110, client devices 120, and external systems 130, or organized in any other suitable manner.

In some embodiments, the online system 110 can be implemented in a network-addressable computing system that can host one or more components for generating, storing, receiving, and sending data (e.g., content-related data, user-related data, data related to entities associated with various therapeutics, etc.). The online system 110 can thus be accessed by the other components of the system 100A either directly or via network 140 described below. In particular embodiments, the online system 110 can include one or more servers (e.g., unitary servers, distributed servers spanning multiple computers or multiple datacenters, etc.). The servers can include one or more server types (e.g., web server, messaging servers, advertising servers, file servers, application servers, exchange servers, database servers, proxy servers, etc.) for performing functions or processes described. In particular embodiments, each server can thus include one or more of: hardware, software, and embedded logic components for carrying out the appropriate functionalities associated with the method(s) described below.

In some embodiments, the client device(s) 120 function to deliver the behavioral therapy and/or adaptive interventions generated and/or stored by the online system 110 to patients exhibiting GI and/or inflammatory health condition symptoms in a timely manner. The client device(s) 120 can include computing components, input devices, and/or output devices providing interfaces for receiving patient inputs and transmitting digital content data and/or sensor-derived data over the network 140 (described in more detail below). In some embodiments, the client device(s) 120 can include one or more of: mobile computing devices (e.g., a smartphone a personal digital assistant); a conventional computing system (e.g., desktop computer, laptop computer); a tablet computing device; a wearable computing device (e.g., a wrist-borne wearable computing device, a head-mounted wearable computing device, an apparel-coupled wearable computing device); a toilet-interfacing computing device; and any other suitable computing device.

In some embodiments, the client device(s) 120 can be configured to store and/or execute an application (e.g., mobile application, web application) that allows a user of the client device 120 to interact with the online system 110 by way of the network 140, in order to receive digital content associated with one or more therapeutic interventions and/or provide data associated with survey responses, sensor-derived data associated with interactions with such interventions, and/or any other suitable data. In relation to providing treatments, in some embodiments, the client device(s) 120 can include operation modes for administering treatments to the user (e.g., in relation to providing prescription digital therapeutics upon diagnosis of the GI and/or inflammatory health condition of the user, in relation to providing medications, in relation to providing pain management therapies, etc.).

In some embodiments, the external system(s) 130 function to transmit data (e.g., 3^{rd} party data) and/or receive data (e.g., 3^{rd} party data) associated with therapeutic interventions and/or user data (e.g., patient data). The external system(s) 130 can include systems associated with electronic health records (EHRs) of the patient(s), systems associated with collection and/or storage of patient data (e.g., biometric data, behavioral data, social network data, communication data, etc.), systems associated with care providers (e.g., health insurance providers, health care practitioners, etc.), and/or any other suitable systems. In some embodiments, the external system(s) can provide applications for communicating data in a manner that is protective of personal health information (PHI) and/or other sensitive patient data. Additionally or alternatively, the external system(s) can be associated with 3^{rd} party content generators and generate digital content in visual formats, audible formats, haptic formats, and/or any other suitable formats.

In some embodiments, the external system(s) 130 and/or client device(s) 120 can be configured to interact with the online system 110 by way of an application programming interface (API) executing on a native operating system of the external system(s) 130 and/or client device(s), in order to access API-associated data associated with the therapeutic interventions, patient health records, and/or other data (e.g., biometric data, patient behavior data through social networks, communication data through communication subsystems, etc.).

As indicated above, in some embodiments, the external system(s) 130 and/or client devices 120 can further include sensing components configured to generate data from which patient biometrics and/or behaviors can be extracted. In relation to biometric data, the external system(s) 130 and/or client devices 120 can include sensing components associated with one or more of: activity of a patient (e.g., through accelerometers, gyroscopes, motion coprocessing devices, etc.); facial expressions of the patient (e.g., through eye tracking, through image/video processing) for determination of cognitive states (e.g., associated with depression, anxiety, emotions, etc.) and/or performance of activities and/or interacting with content provided through the intervention regimen; physiological and/or psychological stress of a patient (e.g., in relation to respiration parameters, in relation to cardiovascular parameters, in relation to galvanic skin response, in relation to neurological activity, in relation to other stress biometrics, etc.); sleep behavior of a patient (e.g., with a sleep-monitoring device); digestive health of a patient (e.g., in relation to microbiome composition, in relation to stool-based assays, in relation to urine-based assays, in relation to smart-pill devices, in relation to smart toilet devices); and any other suitable sensors or devices from which biometric signals can be acquired for assessment of patient health.

In relation to behavioral data, in some embodiments, the external system(s) 130 and/or client devices 120 can include components for extracting behavioral data associated with communications and social behavior, which can be indicative of changes in patient health associated with different symptoms. Such components can include location sensors (e.g., direct location sensors, location sensing modules based on connections to local networks, triangulation systems, etc.) for tracking user motility and/or other behavior patterns, components associated with API access to social networking data, components associated with messaging communication behavior (e.g., components for accessing SMS or other messaging application data of a patient, with respect to messaging entities, messaging content, etc.), components associated with calling communication behavior (e.g., in relation to inbound/outbound calls, in relation to call duration, in relation to call content, etc.), data from digital assistants (e.g., voice-activated digital assistants) and any other suitable components from which behavioral data can be extracted.

In some embodiments, the network 140 functions to enable data transmission between the online system 110, the client device(s) 120, and the external system(s) 130, in relation to detection of patient states of wellbeing (e.g. with respect to GI and/or inflammatory health condition symptoms). The network 140 can include a combination of one or more of local area networks and wide area networks, and/or can include wired and/or wireless connections to the network 140. The network 140 can implement communication linking technologies including one or more of: Ethernet, worldwide interoperability for microwave access (WiMAX), 802.11 architecture (e.g., Wi-Fi, etc.), 3G architecture, 4G architecture, 5G architecture, long term evolution (LTE) architecture, code division multiple access (CDMA) systems, digital subscriber line (DSL) architecture, and any other suitable technologies for data transmission.

In some embodiments, the network 140 can be configured for implementation of networking protocols and/or formats including one or more of: hypertext transport protocol (HTTP), multiprotocol label switching (MPLS), transmission control protocol/Internet protocol (TCP/IP), file transfer protocol (FTP), simple mail transfer protocol (SMTP), hypertext markup language (HTML), extensive markup language (XML), and any other suitable protocol/format. The network 140 can also be configured for and/or provide, through communication links, encryption protocols for improving security of patient data transmitted over the network 140.

In some embodiments, the system 100A can include or be configured to interface with other system components associated with generation and/or delivery of behavioral therapy and related adaptive interventions. For instance, the system 100A can include or be associated with environmental control devices configured to affect patient states of wellbeing passively or actively, in relation to the intervention types described in more detail below. In some embodiments, such devices can include environmental control devices, including one or more of: lighting control devices, audio output devices, temperature control devices, and any other suitable environmental control devices. The system 100A can coordinate operation of such devices with delivery of adaptive interventions to patients, such that aspects of the patient's environment can be modulated in coordination with other therapeutic measures to improve patient wellbeing in relation to GI and/or inflammatory health condition symptoms. For instance, in variations of the method below, the system 100A can include and/or communicate control instructions for devices in the environment of the patient, in order to facilitate control of pain volume, in relation to magnitude of pain/intensity of pain (e.g., by focusing the user on real time environmental changes) and/or to cause improvements in lives of patients in another suitable manner.

As such, in one embodiment, the system 100A can include an output device (e.g., component of client device 120, component of external system 130, etc.) that functions as an environmental control device in an environment of the patient, where the processing subsystem further includes instructions for adjusting the operation mode in coordination with monitoring a change in symptoms (e.g., pain symptoms) of the patient. Modulation of output device operation modes can thereby produce an adjustment in symptoms (e.g., pain volume) associated with the condition of the patient. In various embodiments, the environmental control device can modulate one or more of: an audio output, a thermal parameter adjustment, a visually-observed output, a haptic output, and a light output in the environment.

In some embodiments, the system 100A can include an output device (e.g., component of client device 120, component of external system 130, etc.) that functions as a communication device for transmitting communications between the patient and an entity associated with the patient, where the processing subsystem further includes instructions for generating a scripted communication for transmission to an entity associated with the patient, in coordination with monitoring a change in a physiological symptoms of the patient.

In some embodiments, the system 100A can be configured to interface or include any other suitable system components. Embodiments, variations, and examples of one or more components of the system 100A described above can implement one or more embodiments, variations, and examples of the methods 200A, 200B, and/or 200C, as described below. The system 100A can additionally or alternatively be configured to implement other methods.

### Production Environment

FIG. 1B depicts a block diagram of a production environment 100B for treating gastrointestinal and inflammatory health conditions using prescription digital therapeutics, according to one or more embodiments.

As shown in FIG. 1B, in one embodiment, production environment 100B includes PDT computing environment 141, patient 142, and patient computing systems 144. In various embodiments, production environment 100B optionally includes patient monitoring devices 146, health practitioner 148, and/or health practitioner computing systems 150. In various embodiments, production environment 100B includes communications channels 143, which facilitate communication between PDT computing environment 141 and one or more of patient computing systems 144, patient monitoring devices 146, and health practitioner computing systems 150.

In one embodiment, PDT computing environment 141 includes PDT user interface 150, patient monitoring system 152, patient condition determination system 166, personalized regimen generation system 168, content selection system 170, and module gating system 172.

In one embodiment, PDT computing environment 141 further includes patient database 156. In one embodiment patient database 156 includes patient profile and pre-assessment data 158, patient condition data 162, patient personalized regimen data 164, and patient interaction data 154.

In one embodiment PDT computing environment 141 further includes therapeutic module database 174. In one embodiment, therapeutic module database 174 includes therapeutic module data 176, which further includes first therapeutic module 178, and second therapeutic module 184 through Nth therapeutic module 190. In one embodiment, first therapeutic module 178 includes module 1 content data 180 and module 1 protocol data 182, second therapeutic module 184 includes module 2 content data 186 and module 2 protocol data 188, and Nth therapeutic module 190 includes module N content data 192 and module N protocol data 194.

In one embodiment, PDT computing environment 141 further includes processor 196 and physical memory 198, which together coordinate the operation and interaction of the data and data processing systems associated with PDT computing environment 141. Each of the above listed elements will be discussed in further detail below.

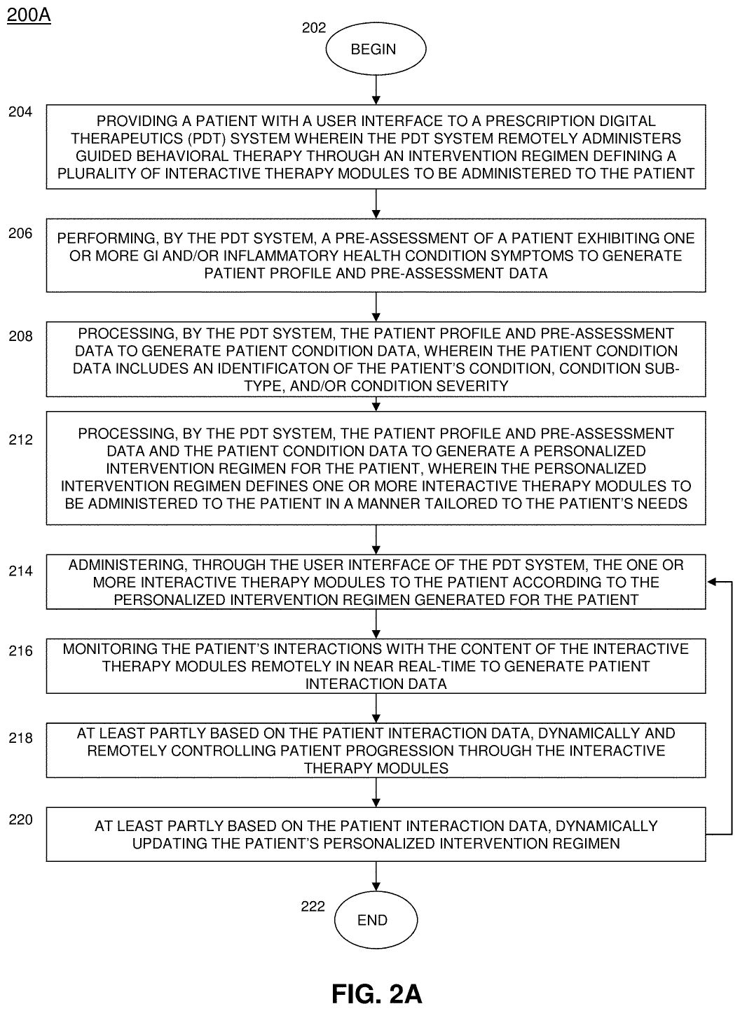

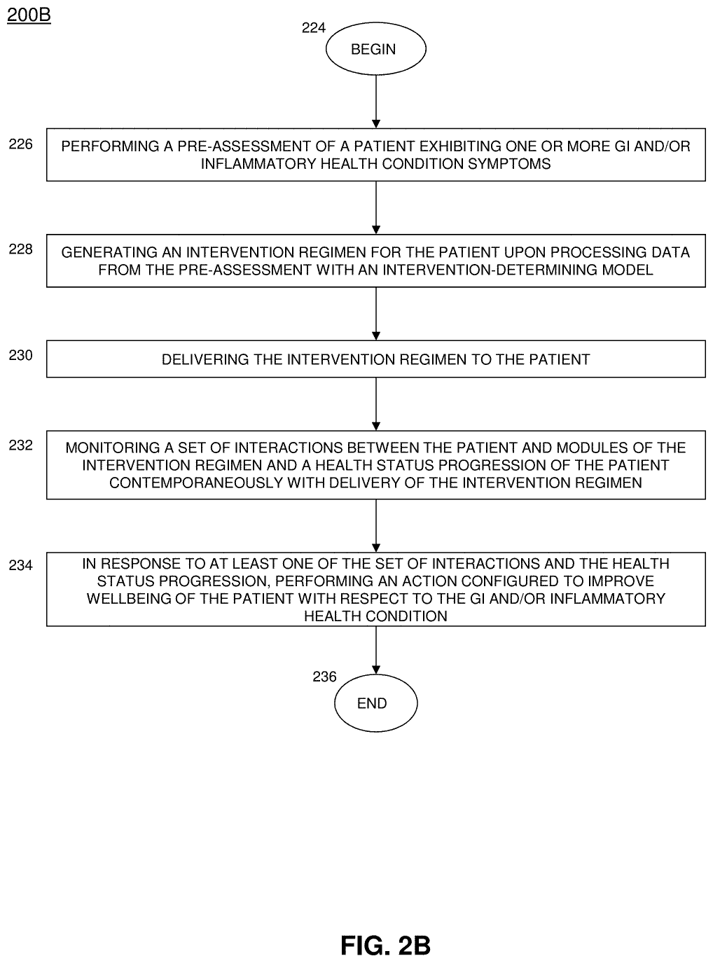

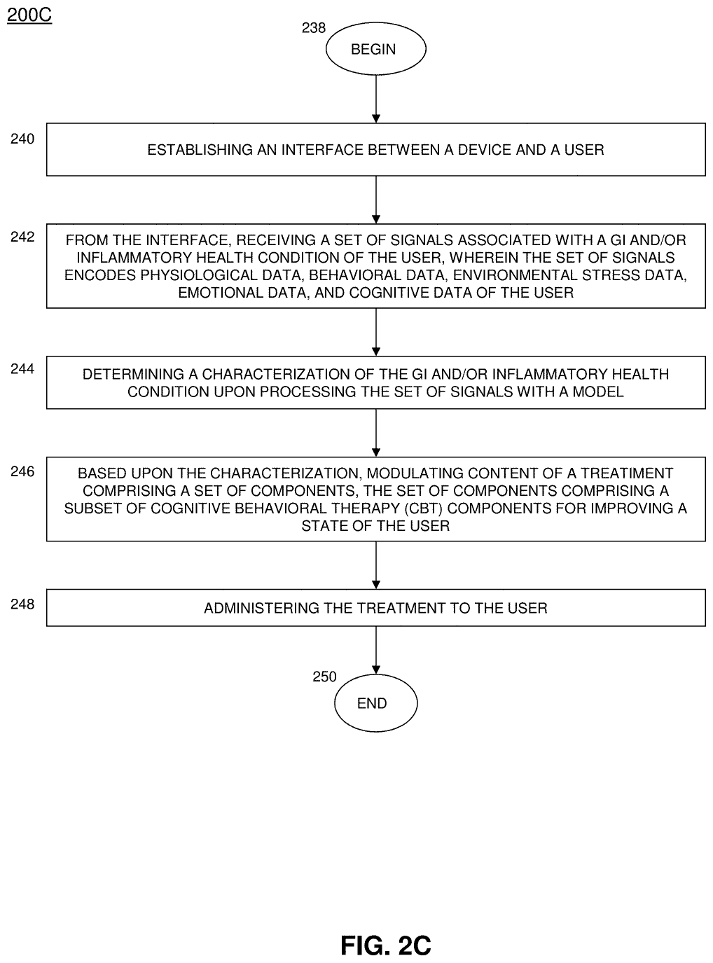

As will be discussed in further detail in relation to the method of FIG. 2A below, in one embodiment, patient 142 is provided with a prescription digital therapeutics (PDT) system, wherein the PDT system remotely administers guided behavioral therapy through an adaptive intervention regimen including a plurality of interactive therapy modules. In one embodiment, the PDT computing environment 141 communicates with patient 142 via one or more communications channels 143 between patient computing systems 144 and a user interface of the PDT system, such as PDT user interface 150.

In one embodiment, therapeutic module database 174 contains a repository of data related to each of the available therapy modules, including module content data and module protocol data. As noted above, a therapeutic protocol defines the rules, syntax, semantics, and synchronization of communications with a patient. In various embodiments, the therapeutic module database may be populated and/or updated periodically by health practitioner 148, for example, through health practitioner computing systems 150.

In one embodiment, module gating system 172 is responsible for determining which parts of the intervention regimen patient 142 has already completed, if any, as well as determining which modules and/or module content should be gated, locked, and/or unlocked. The operation of module gating system 172 will be discussed in additional detail below.

In one embodiment, upon a determination by module gating system 172 that patient 142 has not completed any modules, content selection system 170 may select module 1 content data 180 from first therapeutic module 178 of therapeutic module database 174. Content selection system 170 may then administer module 1 content data 180 to patient 142 through PDT user interface 150. In one embodiment, module 1 content data 180 is administered to patient 142 according to one or more therapeutic protocols defined by module 1 protocol data 182.

In one illustrative embodiment, first therapeutic module 178 may be an introduction and education module, which introduces the patient to the system features, and provides education to the patient relating to the methods utilized by the system and/or relating to the patient's particular disease, disorder, and/or condition. In one embodiment, first therapeutic module 178 also generates patient profile and pre-assessment data 158 by virtue of interaction between patient 142 and the content provided through PDT user interface 150 of the PDT system. In one embodiment, patient profile and pre-assessment data is generated independently of first therapeutic module 178. Additional details regarding first therapeutic module 178 (the introduction and education module) will be provided below.

In one embodiment, upon a determination by module gating system 172 that patient 142 has completed first therapeutic module 178, but has not completed second therapeutic module 184, content selection system 170 may select module 2 content data 186 from second therapeutic module 184 of therapeutic module database 174. Content selection system 170 may then administer module 2 content data 186 to patient 142 through PDT user interface 150. In one embodiment, module 2 content data 186 is administered to patient 142 according to one or more therapeutic protocols defined by module 2 protocol data 188.

In one illustrative embodiment, second therapeutic module 184 may be a physical illness narrative module, which, in some embodiments, solicits narratives from the patient regarding the impact that the patient's disease, disorder, and/or condition has had on their lifestyle, mental state, and overall well-being. In one embodiment, second therapeutic module 184 generates patient illness narrative data (not shown) by virtue of interaction between patient 142 and the content provided through PDT user interface 150 of the PDT system. In one embodiment, second therapeutic module 184 also introduces the patient to the concept of a personal disease model, and guides the user through the process of creating a personal model. In various embodiments, second therapeutic module 184 solicits additional data from the patient for use in creation of the personal model, such as data related to the patient's counter-productive behaviors, unhelpful thoughts, and negative emotions. Additional details regarding second therapeutic module 184 (the physical illness narrative module) will be provided below.

In one embodiment, patient profile and pre-assessment data 158 is processed by patient condition determination system 166 of the PDT system to generate patient condition data 162, as will be discussed in additional detail below.

In one embodiment, once patient profile and pre-assessment data 158 and patient illness condition data 162 have been generated, personalized regimen generation system 168 utilizes patient profile and pre-assessment data 158 and patient condition data 162 to generate a personalized intervention regimen for the patient, which is represented in FIG. 1B by patient personalized regimen data 164. In various embodiments, patient personalized regimen data 164 includes data representing regimen details such as, but not limited to, which of the available remaining therapy modules to administer to the patient, in what order to administer the therapy modules, a time schedule for when/how often to administer the therapy modules, what content to include in each of the therapy modules, and how to present the therapy module content to the patient. Additional details regarding generation of a personalized intervention regimen for the patient will be discussed below.

In one embodiment, once the personalized intervention regimen for the patient has been generated, patient personalized regimen data 164 is provided to module gating system 172 to determine which components of the intervention should be gated, locked, or unlocked, and content selection system 170 may then administer content data related to the appropriate therapeutic module to patient 142 through PDT user interface 150.

In one embodiment, as the patient is progressing through the personalized intervention regimen and interacting with the content of the therapeutic modules, the patient's interactions with the module content may be monitored remotely, either at fixed intervals, or in near real-time. As will be discussed in additional detail below, there are a variety of ways to remotely monitor a patient's interactions with the module content, such as through a patient monitoring system 152 of the PDT system, or through external patient monitoring devices 146, such as sensors, etc., which may then transmit patient data and/or patient interaction data 154 over one or more communications networks 143.

In one embodiment, based on the patient interaction data 154, the progression of the user through the through the therapeutic modules may be dynamically and remotely controlled, for example, though a system such as module gating system 172. As one illustrative example, module gating system 172 may be programmed to gate, lock, or unlock various modules and module components at set intervals. If the patient interaction data 154 indicates that the user would benefit from shorter or longer intervals between lesson modules, module gating system 172 may dynamically adjust how often to unlock new content. A more detailed description of the module gating system used to dynamically and remotely control patient progression through the modules will be provided below.

In one embodiment, based on the patient interaction data 154, the patient's personalized intervention regimen may be updated or changed. For example, user input in one module might change the recommendation for how to present subsequent modules. In the case where the patient is being remotely monitored in near real-time, this allows for the personalized intervention regimen to be dynamically adaptive, thus resulting in administration of the guided therapy in a manner that is most efficient and effective for the patient. Additional details regarding dynamically updating the patient's personalized intervention regimen will be discussed in further detail below.

### Methods Overview

FIG. 2A depicts a flowchart of a method 200A for treating gastrointestinal and inflammatory health conditions using prescription digital therapeutics, according to one or more embodiments.

As shown in FIG. 2A, in one embodiment, the method 200A can include operations for: providing a patient with a user interface to a prescription digital therapeutics (PDT) system wherein the PDT system remotely administers guided behavioral therapy through an intervention regimen defining a plurality of interactive therapy modules to be administered to the patient 204; performing, by the PDT system, a pre-assessment of a patient exhibiting one or more GI and/or inflammatory health condition symptoms to generate patient profile and pre-assessment data 206; processing, by the PDT system, the patient profile and pre-assessment data to generate patient condition data, wherein the patient condition data includes an identification of the patient's condition, condition sub-type and/or condition severity 208; processing, by the PDT system, the patient profile and pre-assessment data and the patient condition data to generate a personalized intervention regimen for the patient, wherein the personalized intervention regimen defines one or more interactive therapy modules to be administered to the patient in a manner tailored to the patient's needs 212; administering, through the user interface of the PDT system, the one or more interactive therapy modules to the patient according to the personalized intervention regimen generated for the patient 214; monitoring the patient's interactions with the content of the interactive therapy modules remotely in near real-time to generate patient interaction data 216; at least partly based on the patient interaction data, dynamically and remotely controlling patient progression through the interactive therapy modules 218; and at least partly based on the patient interaction data, dynamically updating the patient's personalized intervention regimen 220.

In various embodiments, method 200A functions to educate users regarding treatment and therapy options in the context of improving symptoms associated with a variety of health conditions; detect, in real or near-real time, states of health condition symptom severity in non-invasive manners; and administer therapeutic interventions in a customized, and adaptive manner to one or more patients exhibiting health condition symptoms. In some embodiments, the method 200A can be used to provide tailored behavioral therapy to patients in an adaptive and customizable manner. Method 200A will be discussed in additional detail below.

In some embodiments, providing guided behavioral therapy and skills training includes providing adaptive interventions for patients with gastrointestinal (GI) and/or inflammatory health conditions.

FIG. 2B depicts a flowchart of a method 200B for providing adaptive interventions for gastrointestinal and inflammatory health conditions, according to one or more embodiments.

As shown in FIG. 2B, in one embodiment, a method 200B can include operations for: performing a pre-assessment of a patient exhibiting one or more GI and/or inflammatory health condition symptoms 226; generating an intervention regimen for the patient upon processing data from the pre-assessment with an intervention-determining model 228; delivering the intervention regimen to the patient 230; monitoring a set of interactions between the patient and modules of the intervention regimen and a health status progression of the patient contemporaneously with delivery of the intervention regimen 232; and in response to at least one of the set of interactions and the health status progression, performing an action configured to improve wellbeing of the patient with respect to the GI and/or inflammatory health condition 234. Method 200B will be discussed in additional detail below.

FIG. 2C depicts a flowchart of a method 200C for providing adaptive interventions for gastrointestinal and inflammatory health conditions, according to one or more embodiments.

As shown in FIG. 2C, in one embodiment, a method 200C can include operations for: establishing an interface between a device and a user 240; from the interface, receiving a set of signals associated with a GI and/or inflammatory health condition of the user, wherein the set of signals encodes physiological data, behavioral data, environmental stress data, emotional data, and cognitive data of the user 242; determining a characterization of the GI and/or inflammatory health condition upon processing the set of signals with a model 244; based upon the characterization, modulating content of a treatment comprising a set of components, the set of components comprising a subset of cognitive behavioral therapy (CBT) components for improving a state of the user 246; and administering the treatment to the user 248. Method 200C will be discussed in additional detail below.

Methods 200B and 200C function to educate patients regarding treatment and therapy options in the context of improving symptoms associated with GI and/or inflammatory health conditions; detect, in real or near-real time, states of GI and/or inflammatory health condition symptom severity in non-invasive manners; and deliver interventions in a customized, and adaptive manner to one or more users exhibiting GI and/or inflammatory health condition symptoms. In some embodiments, methods 200B and 200C can be used to provide tailored cognitive behavioral therapy (CBT) and/or other therapeutic modalities to patients in an adaptive and customizable manner. While GI and/or inflammatory health condition symptoms are described, variations of the methods 200B and 200C can be adapted for generation and provision of interventions for systems associated with other health conditions.

Aspects of methods 200A, 200B, and 200C, such as provision of components, promotion of interactions with the system, processing of data, performance of analyses (e.g., associated with treatment efficacy, associated with user symptoms, etc.), model refinement, and other aspects can be performed at desired frequencies (e.g., weekly, more often than weekly, less often than weekly). For instance, in relation to triggered interactions with the system, the method can promote interactions more often than weekly (e.g., daily, 2 times a week, 3 times a week, four times a week, five times a week, six times a week, etc.) or less often than weekly, in relation to reinforcement of skills acquired by the patients. Furthermore, received data can be processed in real time, or non-real time. However, the methods 200A, 200B, and 200C can have delivery and processing aspects associated with other suitable frequencies.

The methods 200A, 200B, and 200C can be performed by an embodiment, variation, or example of the system 100A described in above (e.g., in relation to processing subsystem components with instructions stored in non-transitory media and other input/output devices); however, the methods 200A, 200B, and 200C can additionally or alternatively be performed using any other suitable system components.

### Method 200A

As noted above, FIG. 2A depicts a flowchart of a method 200A for treating gastrointestinal and inflammatory health conditions using prescription digital therapeutics, according to one or more embodiments.

As shown in FIG. 2A, in one embodiment, method 200A begins at BEGIN 202, and method flow proceeds to operation 204. In one embodiment, at operation 204, a patient is provided with a user interface to a prescription digital therapeutics (PDT) system wherein the PDT system remotely administers guided behavioral therapy through an intervention regimen defining a plurality of interactive therapy modules to be administered to the patient.

In various embodiments, a patient may consult with one or more healthcare practitioners regarding symptoms that the patient is experiencing, and the healthcare practitioner may determine that the patient is suffering from one or more health-related conditions. As noted above, it has been shown by a variety of studies that some health-related conditions can be managed or alleviated through administration of a variety of therapies, such as, but not limited to, psychotherapy, cognitive behavioral therapy (CBT), acceptance commitment therapy (ACT), dialectical behavioral therapy (DBT), mindfulness-based cognitive therapy (MCBT), exposure therapy, hypnotherapy, experiential therapy, and psychodynamic therapy. In various embodiments, components of the above listed modalities may be combined to form a hybrid type of therapy. For example, a hybrid therapy may utilize particular components taken from CBT, ACT, and DBT, wherein the components are selected based on the specific needs of the patient.

Those skilled in the art will be aware that behavioral therapy, and in particular cognitive behavioral therapy (CBT), has traditionally been considered effective for treatment of psychological conditions such as, for example, alcohol and drug use problems, anxiety disorders, depression, eating disorders, emotional trauma, grief or loss, marital or other relationship problems, mental illness, obsessive-compulsive disorder, pain, phobias, post-traumatic stress disorder (PTSD), schizophrenia, sexual disorders, sleep disorders, etc. Additionally, behavioral therapy has traditionally involved counseling by a mental health provider such as a psychiatrist, psychologist, or other provider; typically, behavioral therapy provides a structured format and a limited (i.e., finite) number of sessions. The present disclosure provides new behavioral therapy technologies which may, in some embodiments, be provided to an individual via non-human interactions, such as via a computer-based system. In some embodiments such computer-based systems are designed to mimic portions of interactions, such as useful exercises, assessments, and techniques that may traditionally be carried out in the context of counseling sessions with a mental health provider and/or via exercises recommended thereby (e.g., ‘homework,’ such as values inventories, journaling exercises, self-assessments, and the like). Alternatively or additionally, in some embodiments, provided behavioral therapies (e.g., cognitive behavioral therapies) may be useful in the treatment of certain physiological conditions, such as, but not limited to, inflammatory and/or GI health conditions (e.g., for example, IBS, IBD, etc.).

In one embodiment, upon a determination by a healthcare practitioner that the patient is likely to benefit from administration of a guided behavioral therapy intervention regimen, the doctor may prescribe a therapeutics system to the patient. In some embodiments, the therapeutics system is a prescription digital therapeutics (PDT) system. As noted above, a PDT system differs from traditional computer-based wellness systems, in that the PDT system is required to be approved by a government agency before it can be marketed for administration to humans. Typically, a PDT system requires FDA approval and rigorous clinical evidence to substantiate intended use and impact on disease state. Further, a PDT system is typically a system for which a medical prescription is required for administration to patients.

In one embodiment, once a patient is provided with a prescription digital therapeutics (PDT) system at operation 204, method flow proceeds to operation 206. In one embodiment, at operation 206, the PDT system performs a pre-assessment of a patient exhibiting one or more GI and/or inflammatory health condition symptoms to generate patient profile and pre-assessment data.

In relation to system components described in FIG. 1A above, an embodiment of the online system, in coordination with the network and a client device, can perform the pre-assessment of operation 206 contemporaneously with executing an onboarding process with the patient with the online system. Operation 206 functions to retrieve data describing characteristics of the patient, preferences of the patient, goals of the patient and/or any other suitable patient features that can be used to provide adaptive interventions through an intervention regimen associated with administration of guided behavioral therapy to the patient in a customized and personalized manner.

In relation to patients, in various embodiments, operation 206 can include pre-assessing and onboarding patients to generate patient profile and pre-assessment data, wherein the patient profile and pre-assessment data includes one or more of: demographics (e.g., genders, ages, familial statuses, residential location, ethnicities, nationalities, socioeconomic statuses, sexual orientations, etc.), household situations (e.g., living alone, living with family, living with a caregiver, etc.), dietary characteristics (e.g., omnivorous, vegetarian, pescatarian, vegan, reduced carbohydrate consumption, reduced acid consumption, gluten-free, simple carbohydrate, or other dietary restrictions, etc.), levels of activity, levels of alcohol consumption, levels of drug use, psychological symptom severity, levels of mobility (e.g., in relation to distance traveled in a period of time), biomarker statuses (e.g., fecal calprotectin, cholesterol levels, lipid states, blood biomarker statuses, etc.), weight, height, body mass index, genotypic factors, durations of mindfulness (e.g., mindful minutes), and any other suitable patient characteristic associated with GI and/or inflammatory health conditions.

In one embodiment, patient profile and pre-assessment data includes data, such as, but not limited to, physiological data, behavioral data, environmental stress data, emotional data, and cognitive data of the patient. In relation to mental health associated with GI and/or inflammatory health condition symptoms, in various embodiments, the pre-assessment and/or onboarding process performed in operation 206 can identify mental health statuses of the patient, in relation to comorbid or non-comorbid conditions (e.g., associated with anxiety, associated with depression, associated with social behavior, etc.), where the intervention regimen described in more detail below can be configured to improve mental health states of the patient in a timely and adaptive manner.

In various embodiments, related data can include psychological and/or disease symptom/clinical profile data that informs selection of high priority therapy components, where examples include data such as, but not limited to: illness-related ruminations being predominant; symptoms triggered by anticipatory anxiety; aspects adapted for types of reinforcement based on level of anhedonia, as assessed from system-provided tools associated with depression assessment (e.g., upon identification of anhedonia characteristics of the patient, promoting behavioral activation content by the system and response chaining, where response chaining involves linking of effortful avoided tasks to those that are neutral or slightly rewarding); sources of motivation; reward sensitivity (e.g., sensitivity associated with drive and reward responsiveness (e.g., using a BIS/BAS assessment tool); and threat sensitivity. These types of reward processing can then inform a user's responsivity to progress and failure in goal-pursuit. As such, the method 200A can include receiving a reward sensitivity dataset characterizing motivation and reinforcement behavior of the patient, and modulating aspects of the treatment upon processing the reward sensitivity dataset with one or more models described herein. Mental health, reward tendencies and sensitivity, and motivational aspect identification can, however, be assessed outside of the pre-assessment of operation 206.

In relation to user/patient preferences (e.g., with respect to receiving transmissions associated with the intervention regimen), in various embodiments, the pre-assessment and/or onboarding process performed in operation 206 can identify user preferences associated with scheduling of content delivery (e.g., in relation to frequencies of content delivery described above) associated with one or more aspects of the intervention regimen, preferred formats (e.g., visual formats, audio formats, haptic formats, etc.) of content delivery, frequency of content delivery, location of user when content is delivered, specific device(s) to which content is delivered, and/or any other suitable user preferences.

In relation to assessing goals of the patient, in various embodiments, the pre-assessment and/or onboarding process performed in operation 206 can identify user goals for improving health, in relation to the intervention regimen. Such goals can include one or more goals, such as, but not limited to: reduction of anxiety, reduction of negative emotions, reduction of depression symptoms, improvement of sleep behavior, improvement in socialization, improvement of GI and/or inflammatory health condition symptoms, improvement of medication adherence, improvement in GI and/or inflammatory-related quality of life, improvement of other health condition symptoms, and/or any other suitable goals. Goals can be organized at a high level of abstraction (e.g., improve sleep behavior), and/or at lower levels of abstraction (e.g., improve quality of sleep, reduce number of symptom-induced disturbances to sleep, etc.).

In various embodiments, the patient profile and pre-assessment data can be obtained through various mechanisms, including, but not limited to, from a pre-assessment module of the PDT system, from patient health records accessible by the PDT system, from API access of health monitoring systems through the PDT system, and/or from biometric sensor data obtained by the PDT system from various devices utilized by the patient. Furthermore, patient profile and pre-assessment data can be collected repeatedly throughout performance of the methods described herein, as will be discussed in additional detail below.

In relation to performing the pre-assessment and/or onboarding process, in various embodiments, the online system and/or other system components can implement surveying tools (e.g., to obtain self-report data from the patient) and/or non-survey-based tools for acquisition of data. Survey tools can be delivered through an application associated with the PDT system executing on the client device of the patient and/or through another suitable method, where the survey tools can implement architecture for assessing the patient in relation to mental health, pain, GI and/or inflammatory health symptom severity or disease activity, types of GI and/or inflammatory health condition symptoms, and/or other statuses.

In various embodiments the surveying tools can be derived from one or more patient reported outcome instruments, such as, but not limited to: a Functional Bowel Disorder Severity Index (FBDSI) instrument, a Gastrointestinal Symptom Rating Scale-IBS (GSRS-IBS) instrument, an IBS-Adequate Relief (IBS-AR) instrument, an IBS Global Assessment of Improvement Scale (IBS-GAI) instrument, an IBS Symptom Severity Score (IBS-SSS) instrument, an IBS Quality of Life (IBS-QOL) instrument, a Patient Reported Outcome Measurement Information System (PROMIS) instrument (e.g., Abdominal Pain PROMIS scale, Constipation PROMIS scale, Diarrhea PROMIS scale, etc.), a Visual Analogue Scale for Irritable Bowel Syndrome (VAS-IBS), a patient health questionnaire (e.g., PHQ-9), an anxiety disorder questionnaire (e.g., GAD-7, PC-PTSD, SCARED), a work and social adjustment scale (WSAS)-derived tool, a pain assessment questionnaire (e.g., numerical rating scale, Wong-Baker faces scale, FLACC scale, CRIES scale, COMFORT scale, McGill scale, Color Analog scale, etc.), a clinical disease activity measurement (e.g., CDAI, PUCAI, Mayo Score) and any other tool or instrument.

In various embodiments, survey components can be implemented during pre-assessment of a patient and/or within modules of the intervention regimen, as described in more detail below. In relation to performing the pre-assessment and/or onboarding process at operation 206, in various embodiments, the online system and/or other system components can implement data from devices (e.g., non-survey data). For instance, embodiments of the system can perform pre-assessment with implementation of data from devices including one or more devices, such as, but not limited to: electronic health record-associated devices; wearable devices (e.g., wrist-borne wearable devices, head-mounted wearable devices, etc.) for monitoring behavior and activities (e.g., related to physiological/cognitive stress, related to respiration activity, related to sedentary and active states, etc.) of the user; non-invasive torso-coupled devices (e.g., abdominal or stomach sensors configured to detect GI or digestive activity); ingestible smart-pill devices; smart toilet devices and/or other devices for analyzing stool and/or urine samples from the patient; and other devices. Non-survey-derived data can additionally or alternatively include data derived from API access of social networking platforms, other communication platforms (e.g., for extracting social behavior characteristics associated with text, voice, and other communications of the users), location-determining platforms, and/or other platforms, in order to assess social behaviors of the user.

In some embodiments, multiple surveying tools may be combined in a single assessment module, or may be provided as distinct, separate, assessment modules. Assessment modules may be based on, and used to solicit input for, a variety of diagnostic questionnaires, depending on a particular GI and/or inflammatory health condition that a patient is to be evaluated for. Additionally or alternatively, assessment modules may be designed to evaluate any other physiological and/or psychological conditions of the patient.

In some embodiments, additionally or alternatively, data pertaining to patient symptoms, quality of life, and other medical information may be obtained via access to electronic health records (EHRs) and/or electronic medical records (EMRs). For example, applications in accordance with technologies as described herein may provide for communication with a secure database in order to receive and/or access EHR and/or EMR data for the user. Such data may be used to populate a patient profile, and as a basis for tailoring content, e.g., as described herein.

In one embodiment, once patient profile and pre-assessment data is generated at operation 206, method flow proceeds to operation 208. In one embodiment, at operation 208, the PDT system processes the patient pre-assessment data to generate patient condition data, wherein the patient condition data includes an identification of the patient's condition, condition sub-type, and/or condition severity.

In relation to GI health conditions, in various embodiments, processing of the patient pre-assessment data in operation 208 can result in identification of the patient as having GI health condition symptoms associated with GI health, such as, but not limited to, one or more of: irritable bowel syndrome (MS), inflammatory bowel disease (IBD, such as associated with Crohn's disease or ulcerative colitis), lactose intolerance, gastroesophageal reflux disease (GERD), ulcers (e.g., peptic ulcer disease, gastric ulcers, etc.), functional dyspepsia, hernias, celiac disease, diverticulitis, malabsorption, short bowel syndrome, intestinal ischemia, pancreatitis, cysts, gastroparesis, gastritis, esophagitis, achalasia, strictures, anal fissures, hemorrhoids, proctitis, prolapse, gall stones, cholecystitis, cholangitis, GI-associated cancers, bleeding, bloating, constipation (e.g. chronic idiopathic constipation (CIC)), diarrhea, heartburn, fecal incontinence/encopresis, nausea, cyclic vomiting syndrome, abdominal pain, swallowing issues, weight maintenance issues, and/or any other suitable symptoms.

In relation to inflammatory health conditions, in various embodiments, processing of the patient pre-assessment data in operation 208 can result in identification of the patient as having inflammatory health condition symptoms associated with chronic inflammation, such as, but not limited to, one or more of: diabetes, cardiovascular diseases (e.g., hypertension), liver diseases, arthritis and joint diseases (e.g., rheumatoid arthritis), various allergies, asthma, chronic obstructive pulmonary disease (COPD), and/or any other suitable symptoms.

In various embodiments, operation 208 can further identify one or more sub-types of a GI and/or inflammatory health condition. As one illustrative example, one common type of GI health condition is irritable bowel syndrome (IBS), which can be further classified into a variety of subtypes. Thus, in various embodiments, the PDT system can be configured to receive, automatically detect, or automatically extract information regarding the particular subtype(s) of IBS that a patient has, based on a combination of patient health record data, data collected from health monitoring systems, data collected from biometric sensors, and data collected from patient-reported outcome instruments, in order to prioritize relevant content provided to the patient, in the interests of customizing the program. As specific examples, subtypes of IBS may include IBS-C (with predominant constipation), IBS-D (with predominant diarrhea), and IBS-M (with mixed bowel habits). If the processing operation 208 identifies that the patient is predominantly subtype IBS-C, subsequent portions of the intervention regimen can prioritize content associated more highly with IBS-C. In some embodiments, however, subtype identification may be determined outside of the processing of operation 208.

In addition to identifying type and subtype of a GI and/or inflammatory health condition, in one embodiment, operation 208 of method 200A includes a method of determining severity of the GI and/or inflammatory health condition. For example, in some embodiments, operation 208 can calculate levels of a GI and/or inflammatory health condition-associated marker (e.g., from a sample from the patient, such as a stool sample or a breath sample, from interactions with the system, etc.) to identify the patient as having a certain state of severity (e.g., expression, phenotype, etc.) of the GI and/or inflammatory health condition. As one illustrative example, operation 208 can be implemented through the PDT system executing on a mobile device or other device associated with the patient, where a user interface of the PDT system prompts inputs from the patient pertaining to various symptoms (e.g., pain, defecation, abdominal distension, digestive issues, cognitive symptoms, behavioral effects, etc.) and generates a report indicating severity of the GI and/or inflammatory health condition (e.g., IBS, IBD, etc.).

As one illustrative example, patients who suffer from irritable bowel syndrome (IBS) may be provided with a survey tool, such as the IBS Symptom Severity Scale (IBS-SSS), in which a patient provides grades for five facets of their IBS symptoms: severity of abdominal pain, frequency of abdominal pain, severity of abdominal distention, dissatisfaction with bowel habits, and interference with quality of life. Patient inputs are used to assign a score ranging from 0 to 500, with higher scores indicating more severe symptoms. Based on a patient score, a classification of mild, moderate, or severe IBS may be assigned.

As such, the PDT system disclosed herein can include architecture for receiving data derived from the patient (e.g., through sensor components, through survey components, associated with pain characteristics, digestive characteristics, defecation characteristics, and other characteristics), processing the data with one or more models, and returning scores (e.g., measures of symptom severity, etc.). Scores can also be used for tagging user data with symptom severity, in relation to model aspects and model training/refinement described below.

In one embodiment, once patient condition data is generated at operation 208, method flow proceeds to operation 212. In one embodiment, at operation 212, the patient profile and pre-assessment data and the patient condition data are processed to generate a personalized intervention regimen for the patient, wherein the personalized intervention regimen defines one or more interactive therapy modules to be administered to the patient in a manner tailored to the patient's needs.

In various embodiments, the personalized intervention regimen provides, through client devices, an array of empirically-supported intervention options or actions delivered via a modular and flexible approach, whereby modules of the regimen (a set of overarching principles and evidence-based interventions) can be adaptively provided based on patient states assessed in real-time or near real-time. This allows for individualized treatment planning.

In some embodiments, guided behavioral therapy technologies as provided herein provide a user with a sequence of interactive lesson modules that the user accesses and interacts with via a graphical user interface (GUI) of a PDT system, which may include a web-based application accessible via a web-browser of a user personal computer (e.g., desktop, laptop, etc.), and/or a mobile application, running, at least in part, on and/or accessible via a user mobile computing device. Each interactive lesson module may represent a guided lesson in a particular behavioral therapy skill and includes specific graphical content and/or graphical widgets designed to introduce a user to a particular behavioral therapy skill, such as keeping a symptom diary, managing symptoms, setting goals, identifying and understanding thoughts (e.g., unhelpful and/or irrational thoughts), and the like. Thus, among other things, as a user completes various interactive lesson modules, they are introduced to, and learn to practice, a specific behavioral therapy skill.

In some embodiments, interactive lesson modules are arranged in a particular sequence. For example, a PDT system in accordance with approaches described herein may include controls that encourage and/or require a user to progress through a particular sequence of lesson modules in a prescribed order. For example, the PDT system may restrict access by the user to certain lesson modules, occurring later in the sequence, until others have been completed first.

The order of modules of the intervention regimen provided can vary from patient to patient and/or vary based on other factors (e.g., due to refinement and training of models, as described in further detail below); however, in some embodiments, all patients will have access to and be offered all of the skill modules through PDT systems executing on their respective client devices. The skills-based interventions rely on skill acquisition (initial phase of learning the new skill), then skill practice before proceeding to learn the subsequent new skill (e.g., in one's natural home/social environment). Monitoring of task performance and practicing skills is described in further detail below. In particular, the modules can allow users to develop and train core skills (e.g., 8 core skills, another suitable number of core skills, etc.) associated with understanding their disease, disorder, and/or condition, therapies available, brain-gut connections; relaxation skills; behavioral change, avoidance, and activation; problem solving and coping; pain management; cognitive flexibility; social problem solving and communication; and relapse prevention and skills maintenance.

In some embodiments, sizes of lesson modules—for example, a number of screens a user cycles through, a number of graphical widgets they interact with, an estimated approximate time they are expected to spend with various lesson module(s), etc.—may be tailored to remain relatively small, so as to provide a user ‘bite-sized’ lessons that can and/or are designed to facilitate retention. For example, in some embodiments lesson modules may be designed such that they may be completed with no more than about twenty minutes of continuous user interaction. In some embodiments lesson modules may be designed such that they may be completed with no more than about fifteen minutes of continuous user interaction. In some embodiments lesson modules may be designed such that they may be completed with no more than about ten minutes of continuous user interaction.

Disease, condition, and/or syndrome-specific components include content addressing one or more of: an illness narrative, symptom management for pain and other symptoms, disease-specific psychoeducation, social skills training, and emphasis on GI and/or inflammatory health condition (e.g., IBS-related, IBD-related) cognitions, beliefs, and behaviors. Intervention modules can further include general cognitive behavioral components shared across psychological conditions/disorders such as behavioral activation, attentional processes, relaxation, problem solving, cognitive reframing, and other areas.

In one embodiment, generating a personalized intervention regimen includes one or more of: defining a plurality of interactive therapy modules; processing, through the therapeutics system, the patient profile and pre-assessment data and the patient condition data to identify one or more of the plurality of interactive therapy modules to be administered to the patient; defining a plurality of therapeutic protocols to be utilized in administration of one or more interactive therapy modules; processing, through the therapeutics system, the patient profile and pre-assessment data and the patient condition data to identify one or more of the plurality of therapeutic protocols to utilize while administering the identified one or more interactive therapy modules to the patient; and processing, by the therapeutics system, the identified one or more interactive therapy modules and the identified one or more therapeutic protocols to generate a personalized intervention regimen for the patient. In one embodiment, the therapeutics system is a prescription digital therapeutics (PDT) system.

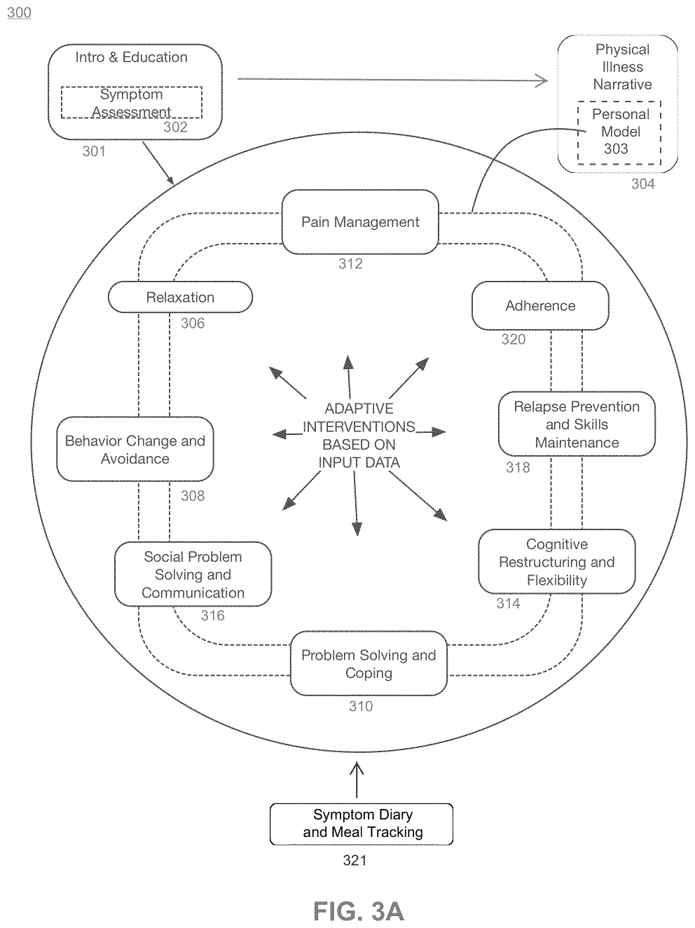

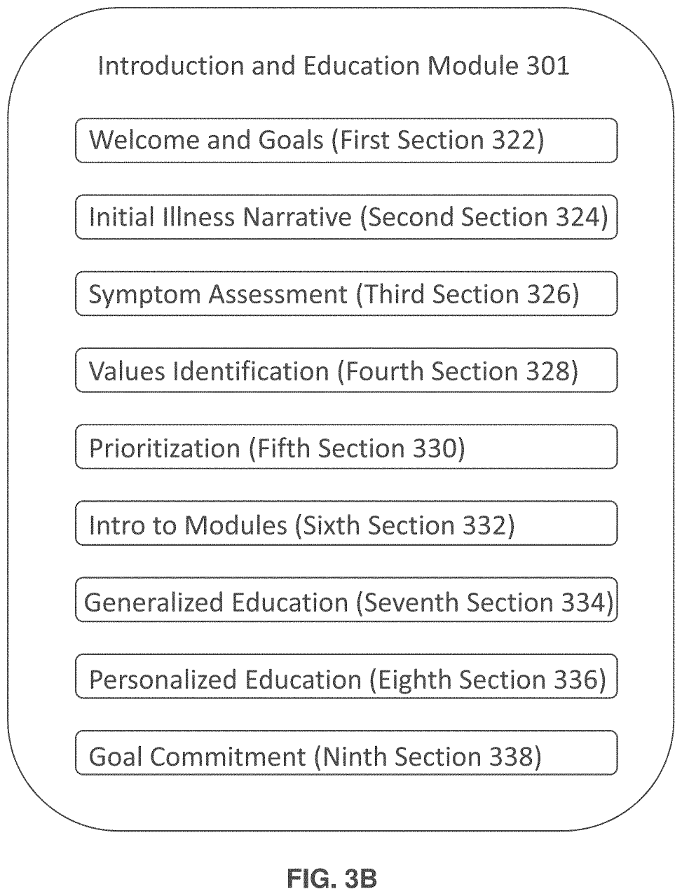

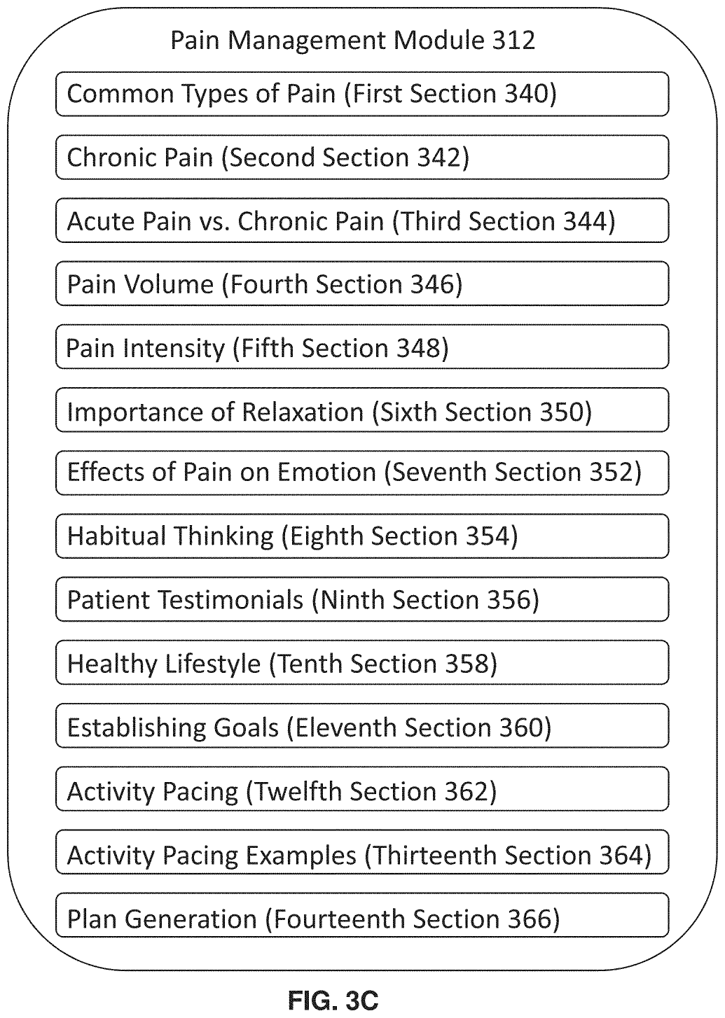

FIG. 3A depicts a schematic of architecture 300 implemented for delivery of intervention regimen components and/or modules, according to one or more embodiments.

As shown in FIG. 3A, in one embodiment, architecture 300 includes introduction and education module 301, which in some embodiments, also includes symptom assessment module 302. In one embodiment, introduction and education module 301 is utilized to generate patient profile and pre-assessment data. In various other embodiments, the patient profile and pre-assessment data is generated and/or obtained outside of the functioning of the introduction and education module 301. In one embodiment, architecture 300 further includes physical illness narrative module 304, which is utilized to generate patient illness narrative data, which in turn, may be utilized for personalization of an intervention regimen, as well as for constructing personal model 303. In one embodiment, the patient profile and pre-assessment data generated by introduction and education module 301, and the patient illness narrative data generated by physical illness narrative module 304 are processed through the PDT system to generate a personalized and adaptive intervention regimen for the patient, as will be discussed in additional detail below.

In one embodiment, the personalized intervention regimen includes one or more additional interactive therapy modules, such as, but not limited to, relaxation module 306, behavioral change and avoidance module 308, problem solving and coping module 310, pain management module 312, cognitive restructuring and flexibility module 314, social problem-solving and communication module 316, relapse prevention and skills maintenance module 318, and adherence module 320. In one embodiment, a symptom diary and meal tracking module 321 is also provided. Each of the above listed modules will be discussed in additional detail below.

In one embodiment, once a personalized intervention regimen is generated at operation 212, method flow proceeds to operation 214. In one embodiment, at operation 214, the one or more interactive therapy modules are administered to the patient through the user interface of the prescription digital therapeutics (PDT) system according to the personalized intervention regimen generated for the patient.

With respect to mechanisms of action, the behavioral and cognitive change interventions described below interrupt the problematic behaviors that are maintaining/perpetuating the targeted symptoms, provide new adaptive coping strategies, and improve perceived control of symptom management in a positive manner. Furthermore, with the adaptive intervention design, the ability to tailor ‘at the right time’ requires relevant information about the user that is used to decide under what conditions to provide an intervention and the appropriateness of the intervention. The following additional interactive therapy modules will be discussed with reference to FIG. 3A. As noted above, inclusion of these therapy modules and/or the order of inclusion of these additional therapy modules, in various embodiments, is dependent on the personalized intervention regimen generated for a particular patient.

While the below modules are described in a particular order, it should be noted that the modules can be performed in any other suitable sequence, omit operations, and/or include additional operations (e.g., based on refinement and training of models described below, and/or based on other factors). Furthermore, aspects of the modules can overlap with each other in any suitable manner.

### Introduction and Education Module 301

In one embodiment of an intervention regimen, introduction and education module 301 focuses on education about the patient's disease and symptoms (e.g., more common symptoms, less common symptoms, etc.). As one illustrative example, an introduction and education module tailored for GI health conditions may provide information regarding methods of diagnosis, promote understanding of functional implications of symptoms in the context of brain-gut axis education (e.g., with effect to the brain's role in gut motility, secretion, nutrient delivery, and microbial balance, and the gut's role in neurotransmitter dynamics, stress and anxiety, mood, and behavior). In general, the introduction and education module is designed to create awareness about what matters to the patient (their reason for trying the program), introduce therapy concepts (e.g., related to CBT, related to other therapies), introduces skills that the user will build by interacting with the system, and assesses user's level of commitment for change.

In one embodiment, an overview of this program links to the patient's specific psychological/disease management challenges. The following points are emphasized: (1) the treatment is modular/flexible in nature and tailored for patient's needs (2) the patient will learn skills, that if practiced, will help them manage their symptoms (e.g., with highlighting of red flag symptoms), improve their quality of life, and lessen the toll that the patient's health conditions take on the patient. Introduction and education module 301 thus can guide the patient to explore the influence that moods, attitudes, beliefs and behavior exert on health and the impact of illness. Introduction and education module 301 can further function to provide tools for education, persuasion (e.g., regarding effectiveness of program completion), personalization, motivation enhancement, setting expectations, eliciting commitment by users, and establishing a relationship between users and the system (e.g., in lieu of a human coach, with supplementation of therapy by a human coach, etc.). Delivery methods for introduction and education module 301 can include one or more of: graphics/animations, metaphorical digital content, interactive exercises provided in a PDT system environment, and a clinical vignette simulating patient-provider interactions.

In various embodiments, introduction and education module 301 may include a variety of individual sections designed to lay a foundation for progression through later modules. While the sections described below are described in a particular order for illustrative purposes, variations of introduction and education module 301 can additionally or alternatively be arranged in another suitable order, omit sections as desired, and/or include additional sections as desired.

FIG. 3B depicts examples of individual sections that may make up an introduction and education module 301 of an intervention regimen, according to one or more embodiments.

As shown in FIG. 3B, in one specific embodiment, introduction and education module 301 includes a First Section 322 configured to welcome the patient and introduce the patient to goals of the intervention regimen delivered through the online system and client device. The First Section 322 is delivered by the system in an interactive format (e.g., with video and text content) that creates a feedback loop with users and processes user responses to tailor subsequent module delivery and content, in order to increase engagement. As such, goals can be set in coordination with user desires, with establishment of collaborative empiricism. Goals can be specific, in terms of detailed planning of what users will do, including frequency, intensity, duration, and context (e.g., where, when, how, with whom, etc.) of the goal(s). Furthermore, in relation to interactive content, introduction and education module 301 can determine topics having greater relevance to the user's current issues (e.g., in relation to comorbid conditions, such as anxiety and depression, in relation to health condition subtypes, such as subtypes of IBS, etc.). In some embodiments, the First Section 322 can include a description of how the program will involve regular practice (e.g., daily, every two days, every 3 days, etc.) of skills (e.g., core skills described above and below), with a guideline for program length (e.g., 8 weeks, less than 8 weeks, more than 8 weeks), and methods of identifying personal progress (e.g., feeling better with mastery of a subset of skills).

In one specific embodiment, introduction and education module 301 includes a Second Section 324 configured to allow the patient to submit information, through a user interface of the PDT system, regarding personal aspects of his/her health condition as an initial physical illness narrative, along with video content to which the patient can compare his/her experiences. Second Section 324 has goals of facilitating emotional awareness, establishing a physical illness narrative that can be revisited as the user gains mastery of skills, and helping the user to articulate and track his/her experiences.

In one specific embodiment, introduction and education module 301 includes a Third Section 326 configured for personalization of subsequent portions of the intervention regimen to the patient, by allowing the patient to indicate, through a user interface of the PDT system, which symptoms (e.g., fatigue, pain, nausea, vomiting, lack of appetite, weight loss, skin problems, eye problems, joint problems, diarrhea, bowel movement issues, cramping pains, bloody stool, medication side effects, other symptoms, etc.) are most bothersome. Third Section 326 can also include architecture for mapping the user's symptoms and health condition-induced factors to various impacts associated with the user's values. In various embodiments, one or more of the mappings can be created, such as, but not limited to: symptoms associated with diarrhea, abdominal pain, urgency, tenesmus, nocturnal bowel movements, rectal bleeding, physical fatigue, and other physical symptoms with mappings to aspects of life (e.g., relationships, work, school, hobbies, daily activities, etc.) that have been affected by such symptoms and the reason such aspects have been affected; medication side effects with mappings to aspects of life (e.g., relationships, work, school, hobbies, daily activities, etc.) that have been affected by such symptoms and the reason such aspects have been affected; social/relationship issues (e.g., stress on loved ones, impacts on friendships, etc.) with mappings to behaviors (e.g., relationships, work, school, hobbies, daily activities, etc.) that have been affected by such symptoms and the reason such aspects have been affected; and behavioral, mental, and emotional factors (e.g., exhaustion, lack of control, inability to perform activities, additional help needed for tasks, limitations in diet, limitations in travel, embarrassment, worry, disease progression, lack of confidence, dwelling thoughts, etc.) with mappings to aspects of life affected and lessons learned with onset of state change.

Third Section 326 has goals for providing education about health condition symptoms and psychological consequences (e.g., behavioral psychological consequences), as well as generating data for future personalization of the intervention regimen.

In one specific embodiment, introduction and education module 301 includes a Fourth Section 328 configured for personalization and values identification, with tools for allowing the user to provide data related to positive and negative changes in his/her life that are attributed to having the health condition, in relation to changes in relationships, levels of embarrassment, curiosity, being understood, stress to self and loved ones, confidence, energy levels, senses of lack of control, worry (e.g., about health issues experienced outside of a comfortable environment, about disease progression, and symptoms, about medication effects, about ability to conduct daily activities, about dietary constraints, about travel, etc.), and other aspects. Fourth Section 328 can also revisit aspects of the user's initial physical illness narrative, with ranking of: symptoms (e.g., physical fatigue, abdominal pain, diarrhea, urgency, tenesmus, bowel movements at night, rectal bleeding, medication side effects, etc.); social/interpersonal factors (e.g., changes to relationships, embarrassment, stress to loved ones, dealing with constant questions about illness, not being understood, etc.); emotional factors (e.g., lack of confidence, mental exhaustion, lack of control, etc.); cognitive factors (e.g., worry about health issues outside of places of comfort, worry about disease progression, catastrophizing, depression, anxiety, other comorbid conditions, etc.); and behavioral factors (e.g., not being able to conduct daily activities, needing to prepare for accidents, dietary restrictions, travel restrictions, etc.).

In one specific embodiment, introduction and education module 301 includes a Fifth Section 330 configured for allowing further customization, by providing the patient with interactive elements that allow the patient to prioritize the order in which content associated with interventions is received.

In one specific embodiment, introduction and education module 301 also includes a Sixth Section 332 configured for introducing subsequent portions/modules of the intervention according to user preferences indicated from outputs of the Fifth Section 330, where the goals of Sixth Section 332 include promotion of treatment credibility (e.g., through presentation of video content by patients having experiences similar to those of the user(s)).

In one specific embodiment, introduction and education module 301 includes a Seventh Section 334 configured for delivery of content for educating the patient about their condition, where the content includes an animated element and audio format content configured to actively interact with the user. The interactive elements function to gauge how well the patient understands the content provided, and to provide additional content to engage and inform the patient depending upon responses of the patient. The Seventh Section 334 has goals of shaping knowledge of symptoms and treatment components of the intervention regimen and enhancing motivation.

As one specific illustrative example, for patients suffering from GI health conditions, Seventh Section 334 can teach users of the system regarding the brain's role in proper gut functioning, and the connection between the mind and the gut. As such, the user can be primed to gain skills related to affecting gut functioning and regulation by changing behaviors, attentional biases, and automatic thought patterns. Seventh Section 334 can further gage internalization and understanding of the user, with provision of further content in this section and/or the eighth section to promote further understanding.

In one specific embodiment, introduction and education module 301 includes an Eighth Section 336 configured for delivery of content for educating the patient in a manner personalized to the patient, where the content includes video and audio format content configured to actively interact with the user, in order to aid the user in understanding influences on the perception of symptoms, based on symptom severity (e.g., related to a threshold level of severity of symptoms, related to fight-or-flight responses, etc.). Eighth Section 336 also provides interactive exercises for learning about physiological-cognitive pathways for perceiving and responding to experienced symptoms and implements architecture for assessing stress and other disease aspects, with implementation of therapeutic techniques for changing reactivity of the brain, thereby decreasing symptom severity.

In one specific embodiment, introduction and education module 301 includes a Ninth Section 338 configured for eliciting commitment from the patient, in relation to different set goals of the patient. The digital content of Ninth Section 338 includes interactive elements for creating a reminder system (according to personalized user preferences and formats for receiving reminders), and interactive elements for setting goals to improve one or more aspects of dealing with the patient's health condition (e.g., with a menu of choices as well as a field for custom user inputs and a field for prompting the user to confirm chosen goals, where example choices can include repeating of tasks, reviewing content, reflecting, identifying entities for social accountability, relocation of application icons on a home screen of a device in a manner that promotes regular use, identifying factors that may obstruct progress, etc.), where the interactive elements allow the patient to confirm when (e.g., specific times), how often, and where the patient will perform activities to meet such goals. The interactive elements further include fields for allowing the patient to set “plan B” options in the event the patient faces obstacles for meeting goals. Finally, Ninth Section 338 includes a brief introduction to subsequent modules of the intervention regimen that are customized to the patient. Ninth Section 338 has goals including setting of expectations, promoting therapeutic persuasiveness, eliciting commitment, increasing user engagement, providing reminders, providing instruction for performing behaviors (e.g., SMART goals).

As noted above, while the sections are described in a particular order above, variations of introduction and education module 301 can additionally or alternatively be arranged in another suitable order, omit sections as desired, and/or include additional sections as desired.

### Physical Illness Narrative Module 304

As noted above, and as used herein, the term “physical illness narrative,” “personal illness narrative,” and/or “patient illness narrative” may include a narrative expressed by a patient regarding the patient's personal experiences with a disease, disorder, and/or condition. An illness narrative is typically a narrative solicited from a patient, which enables a healthcare practitioner to build a more complete picture of the patient's past and present health state in the context of the patient's life, while providing the patient with an opportunity for self-reflection and validation.

In one embodiment of the intervention regimen, physical illness narrative module 304 provides a form of validation (being heard), highlights cognitive distortions/attentional biases and other clinically relevant processes to address, as well as begins the work of emotional exposure. It also provides a point of reference for reflection throughout and at the end of the program. Physical illness narrative module 304 promotes formation of a “personal disease model,” or “personal model” for users, such that they can identify patterns and/or cycles in their disease expression and/or progression, in relation to biology, behaviors, environment, stressors, emotions, and thoughts.

As noted above, and as used herein, the term “personal model,” and/or “personal disease model” may include a construction built based on patient input, which enables the patient to identify stressors, counter-productive behaviors, unhelpful thoughts, and negative emotions as associated with the patient's disease, disorder, and/or condition. A personal model may be utilized to help a patient identify such links, and to consider possible changes in their behavior that could be implemented to address their symptoms.

In various embodiments, during and/or after administration of the second interactive therapy module to the patient through the user interface of the PDT system, second patient response data representing the patient's responses to content provided to the patient through the second interactive therapy module is obtained, for example, through the user interface of the PDT system, or from a variety of patient devices, such as, but not limited to, sensors and/or biometric devices. In one embodiment, the PDT system then processes the second patient response data to generate physical illness narrative data, which can then be used for a variety of purposes.

As one example, in one embodiment, the patient illness narrative data can be utilized to personalize an intervention regimen for the patient, as will be discussed in additional detail below. The patient illness narrative data generated by physical illness narrative module 304 can also be utilized as the basis for formation of a personal model, such as personal model 303 of FIG. 3A, which can then be graphically represented to the user to aid in progression through the intervention regimen.

Referring now to FIG. 2A and FIG. 3A together, key functions of physical illness narrative module 304 can include creation of a patient's personal model 303, validation of a patient's experience, enhancement of self-understanding and illness comprehension, setting the stage for application of behavioral therapy skills to accept uncontrollable elements of physical illness and/or or increase proactivity to address controllable elements of physical illness, and generation of interest for patient engagement.

In some embodiments, prior to soliciting user input for creating a patient's own personal model 303, physical illness narrative module 304 introduces a patient to a process for creating a personal model 303, for example so as to orient them and provide content designed to offer helpful motivation. In some embodiments, graphical content representing educational material is displayed to a patient, for example to introduce them to concept of vicious cycles, and explain how symptoms, stress, and pain can create a feedback loop.

In some embodiments, graphical content corresponding to shared patient experiences and/or testimonials, e.g., in video, written, audio, etc. format, is displayed. Without wishing to be bound to any particular theory, in some embodiments, viewing shared experiences from other patients may help prime a patient to be receptive to therapy, provide motivation, and foster a particular sense of therapy. For example, among other things, such content can serve to reinforce skills that lesson modules present to a patient, by allowing the patient to hear benefits of various lessons and/or skill practice from real patients. In some embodiments, testimonial content can comprise stories from patients describing their experiences living with a particular health condition and which behavioral therapy skills and/or lesson they found particularly helpful. In some embodiments, patients providing videos may be loosely coached, e.g., to structure, direct, etc. their stories in a particular way, while still allowing them to provide authentic, ‘from the heart’ descriptions of their experiences. Additionally or alternatively, among other things, viewing relatable experiences from real patients (e.g., and not actors) can provide a patient with a helpful sense of not being alone in their experiences.

For example, a patient may be prompted to read about another patient's experiences with their condition and guided behavioral therapy approaches such as those described herein. In some embodiments, a patient may view exemplary personal models created by and shared by others. In some embodiments, screens comprising graphical content providing helpful encouragement are displayed within a GUI of the PDT system. Other lesson modules, for example any lesson modules described herein and/or additional lesson modules, providing for development of other behavioral therapy skills provided via the technologies described herein may also include content comprising patient experiences and/or testimonials.

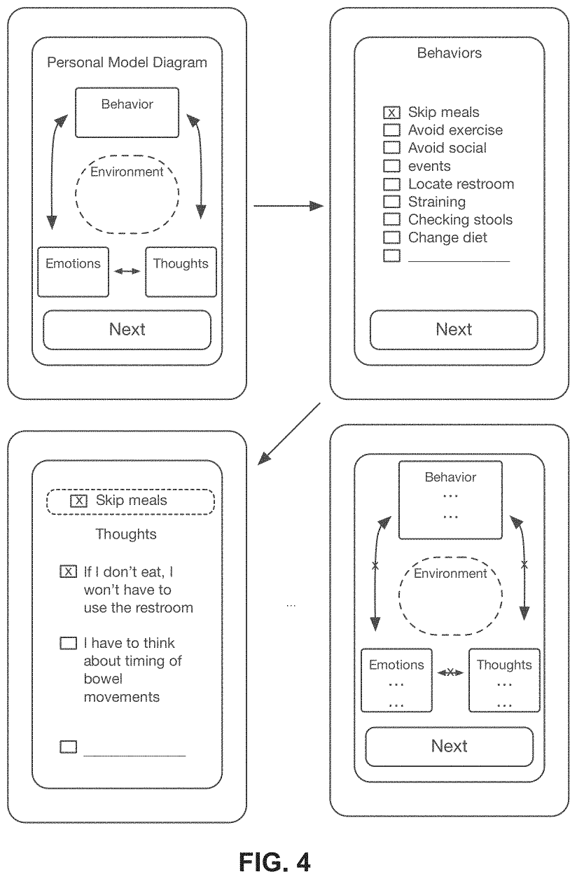

FIG. 4 depicts an example of formation of a personal disease model, according to one or more embodiments.

Referring now to FIG. 3A and FIG. 4 together, in one embodiment, physical illness narrative module 304 can receive patient report data (or other data) regarding the patient's illness history (e.g., painful experiences in a clinical setting, such as with a clinician or hospital environment), thoughts (e.g., thoughts of guilt or responsibility for condition and behaviors, etc.), emotions (e.g., in relation to helplessness, feeling worthless, in relation to embarrassment, etc.), in order to address cognitive distortions for emotional exposure throughout subsequent interactions with the system. In some embodiments, physical illness narrative module 304 is used to implement, via a GUI of the PDT system, a structured process for conveniently soliciting patient input of specific counter-productive behaviors, unhelpful thoughts, and negative emotions that they identify, e.g., in their life and/or as associated with their particular condition for use in creating a patient's own personal model 303.

Additionally or alternatively, physical illness narrative module 304 and/or other related modules can include architecture for prompting the patient to provide data and/or automatically receiving data (e.g., through API access of health monitoring systems, through receiving of sensor signals of devices of the patient, etc.) pertaining to one or more of: biological aspects (e.g., physiological symptoms); behavioral aspects (e.g., in relation to skipping meals, in relation to exercise avoidance, in relation to social event behavior, in relation to locating restrooms, in relation to straining, in relation to checking stools, in relation to other aspects); environmental aspects (e.g., in relation to stress, in relation to temperatures, in relation to diet, etc.); emotional aspects; and thoughts linked to behaviors (e.g., regarding anxiety around diet, regarding to anxiety around performing various activities, etc.),

In one embodiment, physical illness narrative module 304 can include architecture for prompting the patient to provide data and/or automatically receiving data (e.g., through API access of health monitoring systems, through receiving of sensor signals of devices of the patient, etc.) pertaining to one or more aspects such as, but not limited to: pain symptoms, stress symptoms, diarrhea and stool aspects, accidents incurred, constipation and stool aspects, amount of time straining, meals eaten/skipped and times of meals, behaviors and behavioral changes, and other aspects.

In various embodiments, physical illness narrative module 304 may automatically return an analysis summarizing the personal model 303 of the patient (e.g., in a visual format, etc.). Such personalization thus promotes interruption of vicious cycles for patients. As such, in relation to determining characterizations of the health condition of the patient in a personalized manner, the method 200A can include returning a mapping with a network of flows between a set of behaviors specific to the patient, a set of thought patterns specific to the patient, a set of physiological symptoms specific to the patient, a set of emotions specific to the patient, and environmental triggers specific to the patient, where returned outputs of models described can be configured to disrupt flows of the network contributing to deterioration of symptoms of the patient.

In some embodiments, a personal model is constructed as a graphical representation, which comprises text corresponding to patient-selected counter-productive behavior(s), unhelpful thought(s), and negative emotion(s), superimposed on a flow diagram illustrating links between each other. In some embodiments, a personal model graphical representation comprises text corresponding to causes and/or stressors of symptoms. Examples of personal model graphical representations will be provided below in the discussion of the PDT user interface.

As one illustrative example, in some embodiments, generation of a visual or graphical representation of a personal model 303 may include, retrieving, by the illness narrative module 304, stored information previously input by a patient. For example, a patient may have previously provided input identifying causes and/or stressors that impact their particular condition. In some embodiments, a patient provides input corresponding to causes and/or stressors associated with their particular condition via the physical illness narrative module 304. In some embodiments, a graphical representation of a partially completed personal model 303 is rendered, showing the patient identified causes and stressors superimposed on a flow diagram, with portions allocated for graphical representations of additional information such as counter-productive behaviors, unhelpful thoughts, and negative emotions, to be displayed.

Returning now to FIG. 3A, in some embodiments, the physical illness narrative module 304 includes graphical content prompting a patient to review their personal model 303. In particular, in some embodiments, a series of questions (e.g., from a predefined list of questions, e.g., based on a therapeutic protocol) are displayed and presented to the patient along with their personal model graphical representation, prompting the patient to consider their selections, identify links, consider possible changes in their behavior that could be implemented to address their symptoms, and the like. In some embodiments, graphical content, including passages of rendered text, mimicking conversation with a therapist can be displayed. In some embodiments, encouraging graphical content is displayed, and the patient is returned to a home screen.

In some embodiments, delivery methods for the physical illness narrative module 304 can include audio format content and/or textual content for guiding exercises. In some embodiments, physical illness narrative module 304 may be a subcomponent of multiple modules, such that its content can be revisited. For instance, upon development of core skills associated with the modules, the system can trigger revisitation of aspects of physical illness narrative module 304 within the PDT system, such that patients can solidify new skills, reflect on their initial versions of their physical illness narrative and what has changed, generalize skills, maintain skills, and implement cognitive flexibility.

Relaxation Module 306

In one embodiment of the intervention regimen, a relaxation module 306 provides a patient with understanding of what physiological stress feels like (e.g., with education on fight or flight responses) and recognition of the importance of actively optimizing their stress response, particularly because of the connection between stress reactivity, stress hormones and autonomic arousal, and flares in symptoms. Relaxation module 306 informs the patient that (1) stress is a natural reaction and it causes its own physical symptoms (2) the brain does not differentiate between an event that is actually happening to us and an event that we only think is happening, and (3) the connection between stress and flares and symptoms. In one embodiment, relaxation module 306 provides the patient with a rationale for each type of relaxation and how it is tailored for their specific stress symptoms, and provides guided relaxation exercises (e.g., through an application associated with the PDT system executing at the client device). Relaxation module 306 promotes mastery of at least one relaxation technique. Key functions of relaxation module 306 can include decreasing physiological reactivity associated with stress, worry, anxiety, and pain, activation (for depression symptoms), and stress management. Delivery methods for relaxation module 306 can include audio format and/or visual content for guiding exercises associated with targeted muscle groups for progressive muscle relaxation, video-guided demonstration of diaphragmatic breathing, and haptic feedback for exercise guidance.

In one specific embodiment, relaxation module 306 can include video format content that introduces the general concept of relaxation; educates the patient on the applicability of stress-reduction exercises to specific health conditions, with active text boxes that promote user engagement and personalization of the module to the patient's specific symptoms and contexts; addresses common doubts or concerns about relaxation; promotes a guided breathing exercise with a diaphragmatic breathing demonstration and corresponding animated graphic; promotes guided exercises for muscle relaxation using progressive muscle relaxation (PMR) techniques using graphical animations (e.g., of targeted muscle groups); provides information on how relaxation practices can be used (e.g., for abdominal pain, for anxiety, for other stressors, etc.), and encourages practice of exercises by including active interactive elements that the patient can use for scheduling and/or accountability in practicing exercises.

### Behavioral Change and Avoidance Module 308

In one embodiment of the intervention regimen, behavior change and avoidance module 308 provides content covering the importance of activation and approaching avoided situations/experiences in breaking the cycle of persistent pain symptoms and depressive and/or anxious mood. Specific action plans are developed for decreasing avoidance behavior. Key functions of this module can include linking behaviors and mood, mood monitoring (e.g. self-monitoring), activity scheduling, identifying and counteracting avoidance behavior, action planning, activity scheduling, creating anxiety hierarchies, self-monitoring, behavioral experiments, exposure (e.g., imaginal exposure, actual exposure to counteract anxiety) and systematic desensitization for anxiety, coping performance, confidence building, and routine building. Delivery methods for behavior change and avoidance module 308 can include use of automated tailoring for choosing topics that have greater relevance to a patient's current problems (e.g., if a patient reports anxiety, information about physiological responses of anxiety and their relationship with thoughts and behaviors would be more appropriate than information about the physiological symptoms of depression or generic stress).

### Problem Solving and Coping Module 310

In one embodiment of the intervention regimen, problem solving and coping module 310 provides content covering how to differentiate controllable vs. uncontrollable stressors, problem-focused coping (e.g., with problem identification, solution brainstorming, evaluation of solution options, etc.) vs. emotion-focused coping (e.g., with grounding exercises), as well as types of adaptive and maladaptive coping. Mood/anxiety/stress may be managed/ameliorated by using externally-focused coping to distressing and modifiable conditions and internally-focused coping to adjust one's expectations and interpretations for unmodifiable conditions. In various embodiments, problem solving and coping module 310 can include architecture and instructions for promoting practicing of problem solving and coping methods by the patient, such that the patient is better able to handle stronger symptoms (and milder symptoms).

Delivery methods for problem solving and coping module 310 can include digital content with explanations and testimonials of other patients and their uses of problem solving skills, peer support groups facilitated by the PDT system, and other delivery methods.

### Pain Management Module 312

In one embodiment of the intervention regimen, pain management module 312 focuses on awareness of the pain experience, discusses how pain influences mood and vice versa, promotes recognition of certain behaviors (e.g., overactivity, avoidance) and automatic thoughts that may influence pain as well as how to feel more in control of pain by also improving physical and role functioning though increasing adaptive behaviors/coping (attention) and decreasing avoidance/maladaptive behaviors. Key functions of pain management module 312 can include behavioral experimentation, behavior substitution, acceptance of pain, and self-monitoring, with one or more disease-or-syndrome-specific targets. In various embodiments, pain management module 312 can include architecture and content for educating patients regarding re-directing attention away from pain symptoms by focusing on parts of the body that are not in pain, and other methods. In more detail regarding this embodiment, the system can include a processor with instructions stored in non-transitory media that when executed, perform operations for identifying when a patient is in a state of pain, and triggering a response (e.g., verbal cues and instructions to modify attention and/or engage in various pain observation exercises, a change in the environment of the patient, by playing music, by activating a display and providing video or image content, by providing haptic stimulation to the patient, etc.).

In various embodiments, delivery methods for pain management module 312 can include audio format content and/or textual content for managing pain (e.g., with music, exercise, etc.) and/or for promoting attention reconstruction.

FIG. 3C depicts examples of individual sections that may make up a pain management module of an intervention regimen, according to one or more embodiments.

As shown in FIG. 3C, in one embodiment, pain management module 312 can include a First Section 340 that includes content focused on common types of pain (e.g., abdominal pain) associated with the patient's health condition.

In one embodiment, pain management module 312 can include a Second Section 342 focusing on facts about chronic pain associated with the patient's health condition, in relation to constant pain, flare ups of pain, pain signals for people with health conditions vs. without health conditions, factors affecting pain strength, and other factors. Second Section 342 can also include image and video content (e.g., including testimonials of patients similar to the user) and other interactive exercises.

In one embodiment, pain management module 312 can include a Third Section 344 describing differences between acute pain and chronic pain associated with health conditions, and therapies associated with each type of pain.

In one embodiment, pain management module 312 can include a Fourth Section 346 focused on pain volume attributed to specific nerves of the brain, with interactive exercises and content for re-training the brain to adjust pain volume (i.e., pain modulation).

In one embodiment, pain management module 312 can include a Fifth Section 348 focusing on factors that affect pain intensity/perceived pain intensity (e.g., loss of sleep, tense muscles, anxiety, worry, etc.) and methods for modulating pain intensity and duration (e.g., relaxation, distraction, positivity, exercise, medicine, etc.).

In one embodiment, pain management module 312 can include a Sixth Section 350 describing the importance of relaxation in modulating pain volume and creation of a pain management plan.

In one embodiment, pain management module 312 can include a Seventh Section 352 focused on the effects of pain on negative emotions, with architecture for including customized content from the patient's illness narrative (associated with other modules), in a textual, audio, and/or visual format, and allowing the patient to update his/her illness narrative.

In one embodiment, pain management module 312 can include an Eighth Section 354 focused on development of automatic habitual thinking patterns to interrupt and break these negative cycles.

In one embodiment, pain management module 312 can include a Ninth Section 356 with architecture for presenting a patient testimonial regarding a personal experience of catastrophizing thoughts and the effects on worsening mood, pain, and perpetuation of biased attentional processing.

In one embodiment, pain management module 312 can include a Tenth Section 358 focused on promoting a healthy lifestyle to protect the body against stress, pain flares, and other health condition symptoms.

In one embodiment, pain management module 312 can include an Eleventh Section 360, which provides architecture for helping the patient establish goals in various activities in his/her daily life (e.g., school, friendship, sports, etc.), as they relate to pain management.

In one embodiment, pain management module 312 can include a Twelfth Section 362 focused on activity pacing to prevent increases in pain, with interactive content (e.g., derived from patient testimonials, etc.).

In one embodiment, pain management module 312 can include a Thirteenth Section 364 focused on providing examples of activity pacing (e.g., taking breaks during physical exercise, setting limits in relation to pain thresholds, etc.), with interactive modules for setting goals specific to activities that the patient values and/or enjoys.

In one embodiment, pain management module 312 can also include a Fourteenth Section 366 focused on helping the patient to generate a pain management plan with respect to relaxation skills gained (e.g., diaphragmatic breathing, progressive muscle relaxation, etc.), cognitive flexibility skills (e.g., catastrophizing avoidance, etc.), eating and drinking habits (e.g., with respect to regular meals with respect to caffeine limitation, etc.), with respect to activity performing, and with respect to activity pacing.

Returning now to FIG. 3A, the following additional interactive therapy modules are contemplated by the present disclosure.

### Cognitive Restructuring and Flexibility Module 314

In one embodiment of the intervention regimen, cognitive restructuring and flexibility module 314 targets one's interpretations of events/experiences (e.g., how core thoughts influence our feelings and behavior). Cognitive restructuring and flexibility module 314 emphasizes connections between thoughts and physical sensations due to a variety of symptoms. The aim of cognitive restructuring and flexibility module 314 is to teach patients how to identify unhelpful automatic thinking patterns and develop a new pattern of realistic, balanced, and flexible thinking. A health behavior change is targeted in the area of sleep and worry by providing education about worry and how it might interfere with sleep. Strategies to manage worry before bedtime (e.g., use a relaxation practice) are provided as well as basic sleep hygiene. Key functions of cognitive restructuring and flexibility module 314 can include resetting of cognitive distortions (e.g., about self, others, and the world), identification of unhelpful thoughts, challenging of automatic thoughts, creating more balanced thoughts, re-attribution, appraisals of moods, and improving cognitive flexibility. Delivery methods for cognitive restructuring and flexibility module 314 can include a tool providing digital content for reassembling a traditional thought record in which patients enter an unhelpful automatic thought and select from a list of negative thoughts that best matched. After selecting from a list of most common automatic thoughts, the tool can generate a list of possible challenge/alternative thoughts. The patient can then input their own personalized challenge/alternative thought.

### Social Problem Solving and Communication Module 316

In one embodiment of the intervention regimen, social problem solving and communication module 316 provides content promoting effective social behaviors in the context of a variety of health conditions. Social problem solving and communication module 316 can provide tools for one or more of: action planning, social skills training, social support, exposure, and activation, with identification of oneself as a role model, and presentation of information regarding vicarious consequences. In relation to disease- or syndrome-specific targets involving social problem-solving, social problem solving and communication module 316 is intended to assist interactions between patients and their social environment in the context of their health condition(s), and how to communicate effectively about the medical condition/disease. Some examples include requesting support in college (disability services office) or at work; informing a patient that his/her behavior may be an example to others; coping with sense of urgency to use bathroom; and problem solving about bathroom/bowel related challenges and worries. Key functions of social problem solving and communication module 316 can include activation and action planning, problem solving by analysis of factors influencing the behavior and generating strategies to overcome barriers, demonstrating one's ability to cope, decreasing avoidance behaviors, ensuring practice of new coping skills, when symptoms are more severe (e.g., with behavioral rehearsal, etc.). Delivery methods for social problem solving and communication module 316 can include digital content with testimonials of other patients and their uses of problem solving, peer support groups facilitated by the PDT system, and other delivery methods.

In some embodiments social problem solving and communication module 316 can include architecture for triggering actions based on detected changes in symptoms. For instance, in one example, social problem solving and communication module 316 can process data generated by interactions between the user and the system (e.g., with sensor-based monitoring of symptom progression, with user input-based monitoring of symptom progression, etc.), and based upon the data, generate control instructions for recommended actions that would improve social problem solving ability. Examples of recommended actions can include one or more of: guidance for conducting a conversation regarding symptoms (e.g., example language for communicating pain, defecation, or other-related symptoms to an entity, so that the user can experience relief, etc.); triggering automatic communications between the patient and an entity (e.g., automatically sending a private message to a teacher so that the teacher can excuse the patient to manage pain-related, defecation-related, and/or other symptoms); and performing other suitable actions.

### Relapse Prevention and Skills Maintenance Module 318

In one embodiment of the intervention regimen, relapse prevention and skills maintenance module 318 encourages maintenance/continuation of treatment gains, and reinforces positive changes in thoughts and behavior that were accomplished during the active treatment time. Key functions of relapse prevention and skills maintenance module 318 can include skills generalization, skills maintenance, and adaptive monitoring to refresh skills learned. Additionally, relapse prevention and skills maintenance module 318 can perform one or more of: informing patients of signs of relapse into old patterns, development of specific proactive coping tools for future challenges, encouragement of proactive coping for mood regulation, explaining perseverance, education regarding sequential coping strategies, and identification of skills/techniques that were most effective for the user, based on analysis of user outcomes. Delivery methods for relapse prevention and skills maintenance module 318 can include digital content and/or notifications related to monitored states of the patient (e.g., related to relapse) as described in further detail below.

### Symptom Diary and Meal Tracking Module 321

In some embodiments, an intervention regimen may include a symptom diary module that introduces and familiarizes a patient with techniques for tracking their GI and/or inflammatory health condition symptoms. In particular, in some embodiments, technologies described herein provide a convenient interface that facilitates patient tracking and/or monitoring of their GI and/or inflammatory health condition symptoms on a regular basis. In one embodiment, for each day, a patient may use utilize the PDT system disclosed herein to rate pain and stress on a scale, provide ratings characterizing constipation and diarrhea, and provide information characterizing their daily meals and activity routines.

In some embodiments, symptom diary and meal tracking module 321 may include features providing options for setting goals, for example, goals pertaining to a regular timing and/or type of food (e.g., regular consumption of meals, e.g., of a particular and/or consistent size and/or at a particular and/or consistent time) and/or activity (e.g., physical exercise, meditation, breathing and/or relaxation exercises, reflection exercise, e.g., including skills introduced via lesson modules and/or practicable via interaction with one or more other practice modules, such as keeping a regular symptom diary, etc.).

In some embodiments, daily meals may be characterized via use of a streamlined meal-tracking interface. In particular, conventional meal tracking tools typically allow users to input a wide array of information about their particular meals, including detailed information characterizing types of food and/or food content. In contrast, in some embodiments, approaches described herein provide a streamlined meal tracking interface that expressly limits a range of user input and avoids allowing a user to input detailed information regarding meal content. Without wishing to be bound to any particular theory, while such information may be relevant to, for example, identifying allergies, certain food triggers, and ensuring adherence to specific diets, in some embodiments it may not necessarily be of utmost importance for GI and/or inflammatory health condition symptom tracking. Instead, in some embodiments, less finely grained information, such as a broad categorization of meal size (e.g., small, medium, large, as well as an option for whether or not a particular meal was consumed at all), and a particular time at which the meal was consumed can provide sufficient information for tracking meal habits and/or evaluating GI and/or inflammatory health condition symptoms, flare ups, etc. in context of meal habits and/or identifying patterns. Among other things, this approach may facilitate user input of meal information, and can improve adherence to meal-tracking. Moreover, beyond facilitating meal-tracking adherence, without wishing to be bound to any particular theory, this approach encompasses recognition that patients suffering from certain GI and/or inflammatory health conditions and associated mental health conditions, such anxiety and/or depression, can suffer from unhelpful thoughts and compulsive behavior, which conventional, overly complex meal-trackers may exacerbate. Accordingly, among other things, by providing, in some embodiments, a streamlined meal tracking approach, technologies described herein can address unique challenges associated with a particular patient population.

In some embodiments a meal tracking process may provide for display of a streamlined meal tracking interface, that allows for limited user input for each of a limited set of daily meals, such as breakfast, lunch, and dinner. For each meal, a user may be provided with options for input including, for example, an identification of a meal size on a meal size scale, and a meal time. In some embodiments, a meal size scale includes an option for indicating that a particular meal was not consumed at all. In some embodiments, a meal size scale includes four or fewer values—for example, values indicating small, medium, large, along with a value indicating that a meal was not consumed at all. Accordingly, among other things, a streamlined meal tracking interface can be used to solicit and receive user input corresponding to an indication of a meal size and user input corresponding to a meal time. In some embodiments, a streamlined GUI does not provide options for user input of food type, content, or other identification of food. In some embodiments, no other meal-related input is solicited. In some embodiments, once meal tracking data is received, it may be stored and/or provided for display and/or further processing. For example, user inputs may be stored, along with other data, in a daily symptom record populated via a symptom diary.

In some embodiments, the present disclosure provides methods for facilitating meal tracking by a subject suffering from a GI and/or inflammatory health condition, for example including steps of: causing, by a processor of a computing device, display of a streamlined meal-tracking user interface comprising one or more meal tracking features, wherein, for each of one or more particular daily meals, the meal tracking features provide for user input of meal data, including one or more of: binary user input indicating consumption of the particular meal of a day, a size of the particular meal [e.g., via selection of a value on a scale comprising a finite, ordered list of members (e.g., small, medium, and large)], and a time at which the particular meal was consumed. In some embodiments, the meal data input by the user via the meal tracking features is received by the processor stored for display and/or further processing, by the processor, the meal input data.

### Examples of Behavioral Performance Tasks and Assessments

In some embodiments, exercises associated with the intervention regimen can include one or more of: a card sorting task to identify a patient's reinforcers/motivators (e.g., in relation to social reinforcers, reminders, accountability, gaming/competition, responsiveness to quantitative summary feedback, monetary incentives, altruism, learning, elimination of symptoms, etc.); computerized performance tasks (e.g., delayed discounting) to measure/identify salient reinforcers and/or learning style; and performance tasks (e.g., validated distress tolerance computer tasks, tasks associated with mimicked social interactions, etc.) to measure emotional awareness and ability to tolerate various types of distress (psychological, physical, etc.). Aspects of the embodiments and variations described herein can be implemented in coordination with performing a patient pre-assessment (e.g., in relation to non-survey data used for assessments), as described in further detail below.

### Additional or Alternative Interventions

While some intervention types and associated content are described above, in some embodiments, administration of an intervention regimen can further include administration of other interventions, by way of the online system in coordination with other devices, where monitoring of performance of activities with such interventions is described below. Such interventions can include one or more of: anti-inflammatory pharmacologic therapies (e.g., 5-aminosalicylic acid derivatives), corticosteroids, immunomodulators, biologics, nutritional therapies (e.g., enteral nutrition), natural products, whole system medicine (e.g., Eastern Medicine, Ayurveda), mind-body interventions (e.g., yoga, clinical hypnosis), psychotherapy, acceptance and mindfulness-based therapies, biofeedback (e.g., for control of the autonomic nervous system, for control of the cardiovascular system) using biofeedback devices for treating abdominal pain and other symptoms, and other interventions that can be delivered using associated devices.

While the above modules are described in a particular order, it should be noted that the modules can be performed in any other suitable sequence, omit operations, and/or include additional operations (e.g., based on refinement and training of models described below, and/or based on other factors). Furthermore, aspects of the modules can overlap with each other in any suitable manner.

Referring again to FIG. 2A, in one embodiment, once one or more interactive therapy modules are administered to the patient at operation 214, method flow proceeds to operation 216. In one embodiment, at operation 216, the patient's interactions with the content of the interactive therapy modules are monitored remotely in near real-time to generate patient interaction data.

In relation to system components described above, an embodiment of the online system, in coordination with the network and a client device, can monitor the patient's interactions with the content of the interactive therapy modules contemporaneously with administration of the intervention regimen. Monitoring patient interactions functions to provide intimate understanding of progress of the patient in achieving health goals, and to provide further personalization of and administration of intervention content at appropriate times, in order to maintain or improve progress of the patient. Monitoring is preferably performed in near-real time or real time, such that actions can be taken to adjust interventions to patient states according to just-in-time adaptive intervention (JITAI) protocols. However, monitoring can be performed with any suitable delay (e.g., in relation to achieving better accuracy of assessed states of the patient).

In some embodiments, monitoring can be performed using survey components, such as the patient reported outcome instruments described above, which may be delivered with interactive interventions of the intervention regimen, where the patient is prompted and provided with interactive elements that allow the patient to provide self-report data indicating progress statuses. Monitoring can additionally or alternatively be performed with processing of other non-survey data streams, where the non-survey data streams are associated with system or device usage metrics, social networking behavior extracted from usage of social networking platforms and communication platforms, sensor-derived data, and/or other data. Monitoring can thus occur with any frequency and/or level of intrusiveness.

In some embodiments, operation 216 can process monitoring data (e.g., real time data, non-real time data, dynamic data, static data) with a predictive model that outputs indications of one or more of symptom severity predictions, predictions of patient states, indications of predicted success of the patient in achieving goals, and/or other predictions, where training of the predictive model with training sets of data is described in additional detail below.

In one embodiment, ecological momentary assessments of the patient can be used for monitoring. Additionally or alternatively, in one embodiment, client device usage parameters can be used for monitoring. Examples of client device usage parameters can include frequency of application switching, duration of time spent in association with each application login, screen time parameters, data usage associated with different applications and/or types of applications (e.g., social networking, creative, utility, travel, activity-related, etc.) executing on the client device of the patient, time of day of application usage, location of device usage, and other client device usage parameters.

Additionally or alternatively, in one embodiment, the system can process voice data and/or text communication data of the patient for monitoring and modifying interventions and program aspects. Examples of voice data can include voice sampling data from which emotional states can be extracted using voice processing models. In a related manner, natural language processing of textual data (e.g., from communication platforms, from social networking platforms) of the client device can be used to provide context for behaviors of the patient and/or assess emotional or cognitive states of the patient.

Additionally or alternatively, in one embodiment, electronic health record data can be used for monitoring. For instance, if the patient receives medical care, the online system can be configured to receive a notification providing information regarding the type of care the patient has received, and to use this data for monitoring statuses of the patient.

Additionally or alternatively, the system can include architecture for processing data from other sensors of the client device, devices in the environment of the patient, and/or wearable computing devices can be used for monitoring. Such device data can include activity data, location data, motion data, biometric data, and/or other data configured to provide context to behaviors associated with the health condition of the patient. In one example, motion data from motion of sensors of the client device can indicate that the user is sedentary, and may be experiencing symptoms that can be addressed with components of the intervention regimen. In another example, device usage data can indicate that the patient has been using a particular device (e.g., a tablet device in proximity to the patient, where use does not require extensive motion of the patient), in a fixed location (e.g., from GPS data), and in a prone position (e.g., from motion chip data), and may be experiencing health condition symptoms that can be addressed with components of the intervention regimen.

Thus, in various embodiments, monitoring the patient's interactions with the content of the interactive therapy modules includes obtaining one or more of: patient physiological health data; patient psychological health data; patient condition data; patient symptoms data; patient medications data; patient medication adherence data; patient progress report data; patient system usage data; patient device usage data; patient social networking behavior data; patient voice data; patient textual data; patient activity data; patient location data; patient motion data; and patient biometric data.

As such, active monitoring of patient states can be used to adjust administration of intervention regimen modules in order to appropriately meet the needs of the patient. Other data and combinations of data can, however, be used for monitoring.

In one embodiment, once at least part of the patient's interactions with the content of the interactive therapy modules is monitored to generate patient interaction data at operation 216, method flow proceeds to operation 218. In one embodiment, at operation 218, the patient's progression through the interactive therapy modules is dynamically and remotely controlled at least partly based on the patient interaction data.

As one illustrative example, in one embodiment, a first lesson module is initially displayed to a patient, and the patient interacts with graphical content and/or graphical widgets, for example, reading prepared and rendered text, viewing videos, practicing particular exercises etc. In some embodiments, lesson modules may include a variety of graphical content and/or graphical widgets. Examples of graphical content include, without limitation, rendered text, images, and videos, and may be static and/or dynamic. In some embodiments, graphical widgets may include control elements such as selectable (e.g., clickable, tap-able) buttons (e.g., allowing a user to cycle through screens of graphical content, select particular options, etc.), scroll bars, audiovisual media control elements, input elements, such as sliders, selectable lists (e.g., drop-down lists, lists comprising selectable tiles), radio buttons, text boxes for receiving free-form text input from a user, and the like. In some embodiments, content of lesson modules may also include other content, such as audio content, for example providing for guided meditation, controlled breathing, peaceful background noise, etc. In some embodiments, once a user (e.g. patient) has progressed through a first lesson module, the PDT system may determine that the user has completed the first lesson module. In some embodiments, a user confirms that they have completed a lesson module (e.g., the first lesson module), for example by clicking or tapping a selectable graphical button. In some embodiments, a user progresses through screens of content in the lesson module, in a sequential fashion, eventually reaching a final screen. Accordingly, the PDT system may determine that the user has completed a lesson module based at least in part on their having progressed through each screen of the lesson module. In some embodiments, a user may select a final selectable graphical button, on a final screen, to confirm completion. Alternatively and/or additionally, other inputs suitable, e.g., for touch screen interfaces may be received, such as swipes, flicks etc.

In some embodiments, user interaction with graphical content and/or graphical widgets is monitored to determine completion of the first lesson module, for example in order to evaluate whether and/or an extent to which a user has interacted with certain portions (e.g., particular subsets of graphical content and/or graphical widths, e.g., up to all,) of the lesson module. For example, in some embodiments, graphical widgets such as scroll bars may be monitored, e.g., to evaluate whether and/or an extent to which a user scrolls through an entire block of text, thereby indicating that they have fully read it. In some embodiments, a user may be restricted from progressing through a lesson module if they have not entered input into at least a specific portion (e.g., a particular subset, e.g., up to all) of available graphical widgets.

In some embodiments, once a user has completed a first lesson module, as determined by the PDT system, a second, subsequent lesson module is unlocked and made accessible to the user. Prior to completion of the first lesson module, access to the second lesson module may be restricted, preventing the user from viewing and interacting with its content. Following completion of the first lesson module, the user may choose (e.g., via selection of an icon representing the second lesson module) to begin the second lesson module, and its content may be displayed to the user. Thus, among other things, guided behavioral therapy technologies as described herein may provide for control of progression by a user from one lesson module to a next, for example ensuring the user moves through them in a prescribed order.

In some embodiments, all available lesson modules (e.g., provided by the PDT system) are arranged into a single sequence, such that initially only a single, initial lesson module is accessible, and the user steps through lesson modules one by one, to progress through the single sequence. In some embodiments, lesson modules are arranged into multiple sub-sequences. In some embodiments, such sub-sequences themselves may be ordered with respect to each other (i.e., with respect to other sub-sequences), with accessibility of each sub-sequence (e.g., apart from a first, initial sub-sequence) dependent on the user's completion of all lesson modules of preceding sub-sequences. In some embodiments, multiple lesson modules may be accessible in parallel. For example, arrangement of lesson modules into multiple sub-sequences may provide multiple (e.g., parallel and/or branching) paths that a user may complete. In some embodiments, progression through lesson modules may be arranged in a hierarchical, yet not necessarily entirely sequential fashion. For example, in some embodiments, access to a particular higher level lesson module is restricted until a user has completed one or more lower level lesson modules, for example such that certain lower level lesson modules serve as pre-requisites for one or more higher-level lesson modules.

Accordingly, guided behavioral therapy technologies may provide, among other things, a variety of approaches for controlling user/patient progression through behavioral therapy lessons/skill development. In some embodiments, controlling progression in this fashion can facilitate user adherence to a particular therapeutic protocol that defines a particular order, combination, hierarchical structure, etc. of lessons. Without wishing to be bound to any particular theory, this approach may, in some embodiments, improve and/or be designed to improve user benefit from their practice of behavioral therapy, for example by ensuring that the user is introduced to appropriate foundational techniques before progressing onto more advanced ones.

In some embodiments, guided behavioral therapy technologies described herein include features that allow management of a rate at which a user progresses through a sequence of interactive lesson modules. In particular, in some embodiments, a GUI of the PDT system may present additional screens and content and/or require that additional criteria (e.g., beyond completion of a preceding lesson module) be satisfied before allowing a user to progress on from one lesson module to a next. This additional content and/or criteria to be satisfied, accordingly, can act as a gate, temporarily impeding and/or restricting a user's progress. Accordingly, in some embodiments, guided behavioral therapy technologies as describe herein may slow user progress through lesson modules, for example to avoid rushed completion of modules, facilitate and/or improve retention, etc.

In some embodiments, a gate as described herein may be a soft-gate, which impedes, but does not entirely restrict, a user's progress from one lesson to a next. For example, a soft-gate may allow for continued user progression from one lesson module to a next based on additional received input. A soft-gate may, accordingly, among other things, introduce additional friction that may prompt a user to, for example, take a break, but allow a user to continue onto subsequent lesson modules if they desire. In some embodiments, a gate may be a hard-gate that does not provide the user with an option to progress to a desired lesson module and, accordingly, forces a user to delay progressing on to a next lesson module.

In some embodiments, presence of a gate prior to a second lesson module is based on one or more pre-defined criteria for continued progression. These criteria may indicate a time and/or rate of use, and can be implemented to, for example, prevent a user from racing through lesson module too fast and/or manage amount of content to which they are exposed in a particular period of time. For example, criteria may include a measure of time with respect to a time of completion of a prior lesson module. Such a measure of time may be an elapsed time, or a determination of whether the user attempts to complete more than a predefined number of lesson modules within a particular time window, for example, if a user tries to complete more than one lesson module in a single day. In some embodiments, criteria may include an aggregate usage time by a user of the PDT system, for example so as to prevent a user from excessive continued use of an app. In some embodiments, the pre-defined criteria may be dynamically adjusted in real-time based on analysis of user interactions with the content of the lesson modules. For example, if a user is struggling to complete particular exercises, the pre-defined criteria may be adjusted to require that the user spends additional time on particular lesson modules. In some embodiments, a machine learning module is used to evaluate a user's usage habits with respect to certain criteria for continued progression and determine whether to, for example, insert a soft- and/or hard-gate.

In some embodiments, dynamically controlling patient progression through the interactive therapy modules includes: defining criteria for continued progression through the interactive therapy modules; processing patient interaction data representing the patient's interactions with the content of the interactive therapy modules to determine whether the criteria for continued progression should be dynamically adjusted for the patient; and processing patient interaction data representing the patient's interactions with the content of the interactive therapy modules to determine whether the patient has met the criteria for continued progression through the interactive therapy modules. In on embodiment, upon a determination that the patient has not met the criteria for continued progression through the interactive therapy modules, implementing a soft-gate, wherein the soft-gate introduces friction to impede the patient's progress from one lesson module to a subsequent lesson module. In one embodiment, upon a determination that the patient has not met the criteria for continued progression through the interactive therapy modules, implementing a hard-gate, wherein the hard-gate prevents the patient from progressing from one therapy module to a subsequent therapy module until the patient has met the defined criteria.

In various embodiments, the criteria for continued progression through the interactive therapy modules are based on one or more of: an elapsed time from completion of one therapy module to the next therapy module; an aggregate usage time of the PDT system; the number of therapy modules completed within a specific period of time; an evaluation of the patient's understanding of the content provided in the therapy modules; an evaluation of the patient's psychological state while the therapy modules are being administered; and an evaluation of the patient's physiological state while the therapy modules are being administered.

In one embodiment, the patient may be monitored by any suitable means for monitoring a patient, as discussed herein, and the patient monitoring data may be analyzed to determine whether the patient is in a particular psychological or physiological state. As one illustrative example, biometric sensor data may be analyzed to determine that a patient is particularly anxious, and thus, the criteria for continued progression may be modified, for example, to slow down the pacing of the progression, or any other suitable action may be taken to modify the criteria for progression.

In one embodiment, a machine learning module is utilized to evaluate a patient's usage habits with respect to criteria for continued progression to determine whether to implement a soft-gate or a hard-gate. In one embodiment, user/patient progression through the interactive therapy modules is dynamically controlled in real-time.

In some embodiments, gates may be represented and/or implemented via one or more inter-lesson gate screens that are displayed to a user after they complete a first lesson module and attempt to progress onto a second lesson module. An inter-lesson gate screen may appear after the user completes a first lesson module and before a second module is displayed, thereby impeding and/or restricting progression to the second lesson module.

In some embodiments, inter-lesson gate screen(s) comprise graphical content and/or graphical widgets. Graphical content and/or graphical widgets of inter-lesson gate screens may, for example, be used to encourage a particular user action (e.g., such as taking a break) solicit specific input, or prompt the user to interact with their PDT system in a different manner, for example by reflecting on previous lessons and/or practicing previously learned techniques via one or more practice modules, rather than moving along to a new lesson. Examples of inter-lesson gate screens will be provided below in the detailed discussion of the user interface.

In one embodiment, in addition to the patient's progression through the interactive therapy modules being dynamically and remotely controlled at operation 218, method flow may also concurrently or subsequently proceed to operation 220. In one embodiment, at operation 220, the patient's personalized intervention regimen may be dynamically updated at least party based on the patient interaction data.

In relation to system components described above, an embodiment of the online system, in coordination with the network and a client device can, based on the patient's interactions with the content of the interactive modules, perform an action to dynamically update the patient's personalized intervention regimen. For example, in one embodiment, operation 220 functions to provide further customization of the intervention regimen, in order to improve personalization of delivered content to meet the needs of the patient, in an adaptive manner. Operation 220 can also function to increase engagement between the patient and the intervention regimen, in order to improve effectiveness of provided treatments and increase success of the patient in achieving his/her goals.

In various embodiments, updating the patient's personalized intervention regimen can include one or more of: adjusting order of and/or content of intervention modules provided, where intervention types and content are described above; updating electronic health records (EHRs), personal health records (PHRs), and/or open medical records, for instance by writing to or modifying records whenever new information is generated regarding the user/patient. In some embodiments, operation 220 can additionally or alternatively include functionality for increasing engagement of the patient with respect to interactions with content of the intervention regimen.

In various embodiments, updating the patient's personalized intervention regimen can include providing features for increasing engagement and optimal learning. For example, specific descriptions self-reported by the patient can be used in subsequent portions of the intervention regimen to increase personalization of the intervention to drive engagement. Additionally or alternatively, features for increasing engagement and optimal learning can include features that mimic therapist/healthcare provider, or social group interactions (e.g., patient testimonials, clinician video content, etc.). Additionally or alternatively, features for increasing engagement and optimal learning can include features that link the patient's specific current problems (e.g., from operation 232) and/or challenges faced the patient as a trigger to notify the patient to interact with content of the intervention regimen and recommend appropriate skill for improving health states.

Additionally or alternatively, in some embodiments, updating the patient's personalized intervention regimen to promote patient engagement can be done using one or more of: artificial reality tools (e.g., augmented reality platforms, virtual reality platforms) for reducing depression, anxiety, pain, and/or other symptoms; artificial intelligence-based coaching elements for driving interactions with the patient; smart assistants (e.g., Alexa™, Siri™, Google™ Assistant, etc.) for assisting the patient in relation to task management, gamification elements within intervention regimen-associated applications executing on the client device; gamification elements of other devices (e.g., smart toilet devices having interactive elements, such as buttons that control flushing and other subsystems, for promoting triggering of stool sample tracking in relation to various symptoms); smart pill devices and/or medication-dispensing devices that provide insights in an engaging manner in coordination with intervention regimen modules; adjustment of reinforcement schedules (e.g., in relation to reward sensitivity, positive reinforcement, negative reinforcement, etc.) for providing intervention regimen content to the patient; and other elements for increasing engagement.

Thus, in one embodiment, dynamically updating the patient's personalized intervention regimen includes one or more of: adjusting the order of administration of the interactive therapy modules; adjusting the frequency of administration of the interactive therapy modules; adjusting the mode of administration of the interactive therapy modules; adjusting the content of the interactive therapy modules; adjusting the content size of the interactive therapy modules; adjusting the presentation of the interactive therapy modules; adjusting the layout of the interactive therapy modules; updating the patient's electronic health records; updating the patient's personal health records; updating the patient's open medical records; and increasing personalization of the intervention regimen. In one embodiment, the patient's personalized intervention regimen is updated in near-real time based on the patient's interactions with the content of the interactive therapy modules.

As noted above, in various embodiments, features for updating and personalization of the intervention regimen, as well as for promoting engagement with the intervention regimen can be delivered within modules of the intervention regimen before, during, and/or after monitoring of the patient at operation 216.

In one embodiment, upon update of a patient's personalized intervention regimen at operation 220, method flow may return to operation 214 to continue administering the intervention according to the updated personalized intervention regimen. In one embodiment, upon completion of the intervention regimen, method flow proceeds to END operation 222, and the method 200A for treating gastrointestinal and inflammatory health conditions using prescription digital therapeutics is exited to await new instructions.

### Method 200B

As noted above, FIG. 2B depicts a flowchart of a method 200B for providing adaptive interventions for gastrointestinal and inflammatory health conditions, according to one or more embodiments.

As shown in FIG. 2B, in one embodiment, method 200B begins at BEGIN 224, and method flow proceeds to operation 226. In one embodiment, at operation 226, a pre-assessment of a patient exhibiting one or more GI and/or inflammatory health condition symptoms is performed.

In relation to system components described in FIG. 1A above, an embodiment of the online system, in coordination with the network and a client device, can perform operation 226, performing a pre-assessment of a patient exhibiting one or more GI and/or inflammatory health condition symptoms, contemporaneously with executing an onboarding process with the patient with the online system. Operation 226 functions to retrieve data describing characteristics of the patient, preferences of the patient, goals of the patient and/or any other suitable patient features that can be used to provide adaptive interventions in a customized and personalized manner, in order to promote user engagement with the intervention regimen(s) described in subsequent operations of the method 200B.

In relation to patients, in various embodiments, operation 226 can include pre-assessing and onboarding patients and assessing characteristics including one or more of: demographics (e.g., genders, ages, familial statuses, residential location, ethnicities, nationalities, socioeconomic statuses, sexual orientations, etc.), household situations (e.g., living alone, living with family, living with a caregiver, etc.), dietary characteristics (e.g., omnivorous, vegetarian, pescatarian, vegan, reduced carbohydrate consumption, reduced acid consumption, gluten-free, simple carbohydrate, or other dietary restrictions, etc.), levels of activity, levels of alcohol consumption, levels of drug use, psychological symptom severity, levels of mobility (e.g., in relation to distance traveled in a period of time), biomarker statuses (e.g., fecal calprotectin, cholesterol levels, lipid states, blood biomarker statuses, etc.), weight, height, body mass index, genotypic factors, durations of mindfulness (e.g., mindful minutes), and any other suitable characteristic associated with GI and/or inflammatory health conditions.

In relation to GI health conditions, in various embodiments the pre-assessment and/or onboarding process performed in operation 226 can identify the patient as having GI health condition symptoms associated with GI health, such as, but not limited to, one or more of: irritable bowel syndrome (IBS), inflammatory bowel disease (IBD, such as associated with Crohn's disease or ulcerative colitis), lactose intolerance, gastroesophageal reflux disease (GERD), ulcers (e.g., peptic ulcer disease, gastric ulcers, etc.), functional dyspepsia, hernias, celiac disease, diverticulitis, malabsorption, short bowel syndrome, intestinal ischemia, pancreatitis, cysts, gastroparesis, gastritis, esophagitis, achalasia, strictures, anal fissures, hemorrhoids, proctitis, prolapse, gall stones, cholecystitis, cholangitis, GI-associated cancers, bleeding, bloating, constipation (e.g. chronic idiopathic constipation (CIC)), diarrhea, heartburn, fecal incontinence/encopresis, nausea, cyclic vomiting syndrome, abdominal pain, swallowing issues, weight maintenance issues, and/or any other suitable symptoms.

In relation to inflammatory health conditions, in various embodiments, the pre-assessment and/or onboarding process performed in operation 226 can identify the patient as having inflammatory health condition symptoms associated with chronic inflammation, such as, but not limited to, one or more of: diabetes, cardiovascular diseases (e.g., hypertension), liver diseases, arthritis and joint diseases (e.g., rheumatoid arthritis), various allergies and asthma, and chronic obstructive pulmonary disease (COPD), and/or any other suitable symptoms.

In one embodiment, a set of signals can encode physiological data, behavioral data, environmental stress data, emotional data, and cognitive data of the user, from the pre-assessment, health record access, API access of health monitoring systems, and/or biometric sensors. Furthermore, such signals can be collected repeatedly throughout performance of the methods described, as will be discussed in additional detail below.

In more detail with respect to IBS, in various embodiments, the pre-assessment can be configured to receive information regarding (or automatically detect, or automatically extract, based upon symptoms, etc.) the subtype(s) of IBS (e.g., IBS-C with predominant constipation, IBS-D with predominant diarrhea, IBS-M with mixed bowel habits) a patient has, in order to prioritize relevant content provided to the patient, in the interests of customizing the program. For instance, if the pre-assessment operation 226 identifies that the patient is predominantly subtype IBS-C, subsequent portions of the method 200B can prioritize content associated more highly with IBS-C. Subtype identification can, however, be assessed outside of the pre-assessment of operation 226. Furthermore, in relation to subtype identification, prescription digital therapeutics (PDT) provided by the method 200B and system 100AA can be provided as monotherapies, or as complementary therapies. In more detail, complementary therapies for IBS-C can include one or more therapies, such as, but not limited to: antibiotics, antidepressants, antispasmodics, 5-hydroxytryptamine 4 agonists, over-the-counter laxatives, probiotics, selective C-2 chloride channel activators, and other therapies. In more detail, complementary therapies for IBS-D can include one or more therapies, such as, but not limited to: antibiotics, antidepressants, anti-diarrheal medications, antispasmodics, 5-hydroxytryptamine 4 agonists, probiotics, and other therapies. In more detail, complementary therapies for IBS-M can include one or more of: antibiotics, antidepressants, antispasmodics, probiotics, and other therapies. Complementary therapies can further include one or more of: psychological treatments, hypnotherapy, acupuncture, herbal therapies, oils, and other therapies.

In one embodiment, operation 226 of method 200B includes a method of determining severity of a gastrointestinal (GI) and/or inflammatory health condition.

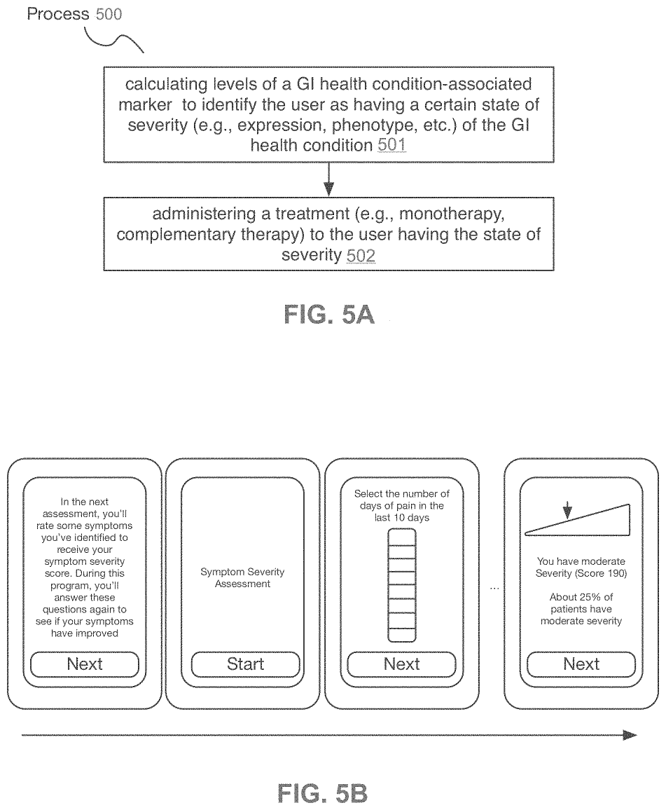

FIG. 5A depicts a flowchart of a process for determining severity of a gastrointestinal and/or inflammatory health condition, according to one or more embodiments.

FIG. 5B depicts examples of a process for determining severity of a gastrointestinal and/or inflammatory health condition, according to one or more embodiments.

Referring to FIG. 5A and FIG. 5B together, in embodiments related to monotherapies and complementary therapies, the process 500, as shown in FIG. 5A, can include operation 501 for calculating levels of a GI and/or inflammatory health condition-associated marker (e.g., from a sample from the user, such as a stool sample or a breath sample, from interactions with the system, etc.) to identify the user (e.g. the patient), as having a certain state of severity (e.g., expression, phenotype, etc.) of the GI and/or inflammatory health condition. As one illustrative example, operation 501 of FIG. 5A can be implemented through a PDT system executing on a mobile device or other device associated with the user, where a user interface of the PDT system prompts inputs from the user pertaining to various symptoms (e.g., pain, defecation, abdominal distension, digestive issues, cognitive symptoms, behavioral effects, etc.) and generates a report indicating severity of the GI and/or inflammatory health condition (e.g., IBS, IBD, etc.), as shown in FIG. 5B.

The process 500 shown in FIG. 5A can then include operation 502, administering a treatment (e.g., monotherapy, complementary therapy) to the user having the state of severity, where the treatment comprises one or more of the therapies described.

Referring back to FIG. 2B, in relation to behavioral therapies vs. other types of therapies, in various embodiments, the process 200B can include adjusting (e.g., decreasing, increasing, maintaining) an amount of a non-behavioral therapy treatment provided to the user based upon the state of severity, and/or correspondingly adjusting (e.g., decreasing, increasing, maintaining) an amount of a behavioral therapy treatment provided to the user, thereby titrating relative treatment types provided to the user based upon returned outputs of models associated with the methods described. As such, a treatment cocktail can include prescription digital therapeutic aspects and non-prescription digital therapeutic aspects.

In relation to mental health associated with GI and/or inflammatory health condition symptoms, in various embodiments, the pre-assessment and/or onboarding process performed in operation 226 can identify mental health statuses of the patient, in relation to comorbid or non-comorbid conditions (e.g., associated with anxiety, associated with depression, associated with social behavior, etc.), where the intervention regimen described in more detail above can be configured to improve mental health states of the patient in a timely and adaptive manner.

In various embodiments, related data can include psychological and/or disease symptom/clinical profile data that informs selection of high priority therapy components, where examples include data such as, but not limited to: illness-related ruminations being predominant; symptoms triggered by anticipatory anxiety; aspects adapted for types of reinforcement based on level of anhedonia, as assessed from system-provided tools associated with depression assessment (e.g., upon identification of anhedonia characteristics of the patient, promoting behavioral activation content by the system and response chaining, where response chaining involves linking of effortful avoided tasks to those that are neutral or slightly rewarding); sources of motivation; reward sensitivity (e.g., sensitivity associated with drive and reward responsiveness (e.g., using a BIS/BAS assessment tool); and threat sensitivity. These types of reward processing can then inform a user's responsivity to progress and failure in goal-pursuit. As such, the method 200B can include receiving a reward sensitivity dataset characterizing motivation and reinforcement behavior of the user, and modulating aspects of the treatment upon processing the reward sensitivity dataset with one or more models described. Mental health, reward tendencies and sensitivity, and motivational aspect identification can, however, be assessed outside of the pre-assessment of operation 226.

In relation to user preferences (e.g., with respect to receiving transmissions associated with the intervention regimen), in various embodiments, the pre-assessment and/or onboarding process performed in operation 226 can identify user preferences associated with scheduling of content delivery (e.g., in relation to frequencies of content delivery described above) associated with one or more aspects of the intervention regimen, preferred formats (e.g., visual formats, audio formats, haptic formats, etc.) of content delivery, frequency of content delivery, location of user when content is delivered, specific device(s) to which content is delivered, and/or any other suitable user preferences.

In relation to assessing goals of the patient, in various embodiments, the pre-assessment and/or onboarding process performed in operation 226 can identify user goals for improving health, in relation to the intervention regimen. Such goals can include one or more goals, such as, but not limited to: reduction of anxiety, reduction of negative emotions, reduction of depression symptoms, improvement of sleep behavior, improvement in socialization, improvement of GI and/or inflammatory health condition symptoms, improvement of medication adherence, improvement in GI and/or inflammatory-related quality of life, improvement of other health condition symptoms, and/or any other suitable goals. Goals can be organized at a high level of abstraction (e.g., improve sleep behavior), and/or at lower levels of abstraction (e.g., improve quality of sleep, reduce number of symptom-induced disturbances to sleep, etc.).

In relation to performing the pre-assessment and/or onboarding process, in various embodiments, the online system and/or other system components can implement surveying tools (e.g., for self-report of data from the patient) and/or non-survey-based tools for acquisition of data. Survey tools can be delivered through an application associated with the PDT system executing on the client device of the patient and/or through another suitable method, where the survey tools can implement architecture for assessing the patient in relation to mental health, pain, GI and/or inflammatory health symptom severity or disease activity (e.g. IBS-symptom severity scale), types of GI and/or inflammatory health condition symptoms, and/or other statuses. In examples the surveying tools can be derived from one or more tools such as, but not limited to: a patient health questionnaire (e.g., PHQ-9), an anxiety disorder questionnaire (e.g., GAD-7, PC-PTSD, SCARED), a work and social adjustment scale (WSAS)-derived tool, a pain assessment questionnaire (e.g., numerical rating scale, Wong-Baker faces scale, FLACC scale, CRIES scale, COMFORT scale, McGill scale, Color Analog scale, etc.), a clinical disease activity measurement (e.g., CDAI, PUCAI, Mayo Score) and any other tool or instrument. Survey components can be implemented during pre-assessment of a patient and/or within modules of the intervention regimen, as described in more detail above. As such, the system can include architecture for receiving data derived from the patient (e.g., through sensor components, through survey components, associated with pain characteristics, digestive characteristics, defecation characteristics, and other characteristics), processing the data with one or more models, and returning scores (e.g., measures of symptom severity, etc.). Scores can also be used for tagging user data with symptom severity, in relation to model aspects and model training/refinement described below.

In relation to performing the pre-assessment and/or onboarding process at operation 226, in various embodiments, the online system and/or other system components can implement data from devices (e.g., non-survey data). For instance, embodiments of the system can perform pre-assessment with implementation of data from devices including one or more devices, such as, but not limited to: electronic health record-associated devices; wearable devices (e.g., wrist-borne wearable devices, head-mounted wearable devices, etc.) for monitoring behavior and activities (e.g., related to physiological/cognitive stress, related to respiration activity, related to sedentary and active states, etc.) of the user; non-invasive torso-coupled devices (e.g., abdominal or stomach sensors configured to detect GI or digestive activity); ingestible smart-pill devices; smart toilet devices and/or other devices for analyzing stool and/or urine samples from the patient; and other devices. Non-survey-derived data can additionally or alternatively include data derived from API access of social networking platforms, other communication platforms (e.g., for extracting social behavior characteristics associated with text, voice, and other communications of the users), location-determining platforms, and/or other platforms, in order to assess social behaviors of the user.

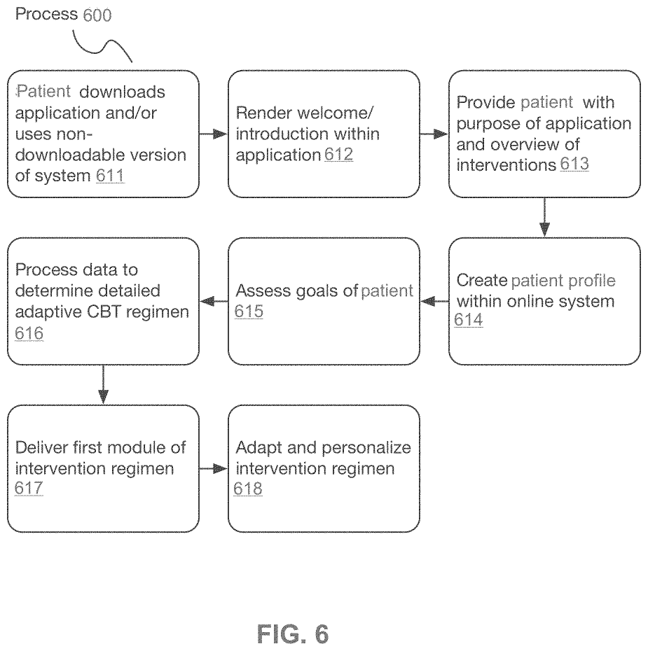

FIG. 6 depicts a flowchart of a pre-assessment and onboarding process of a method for providing adaptive interventions, according to one or more embodiments.

In the illustrative example shown in FIG. 6, the pre-assessment and onboarding process 600 can include operation 611, which facilitates downloading of an application associated with the system and/or using of a non-downloadable version of the system (e.g., via web application, etc.) for delivering the intervention regimen by a client device of the patient; operation 612, which renders a welcome/introduction screen within the application associated with the system; operation 613, which delivers content within the application for educating the patient regarding the purpose of the system and provides an overview of the intervention regimen; operation 614, which creates a patient profile within the online system, resulting in a first tier of personalization by implementing survey and non-survey based tools (e.g., to assess gender, age, preferences for scheduling of content delivery, specific GI and/or inflammatory health condition symptoms of the patient, etc.); and operation 615, which, within the application associated with the system, assesses goals of the patient, resulting in a second tier of personalization. In various embodiments, the second tier of personalization can operate by assessing goals related to anxiety reduction, depression reduction, reduction of IBS and/or IBD or other gastrointestinal (GI) and/or inflammatory disease or syndrome symptoms, improvement of sleep, improvement of socialization, and other goals. In relation to subsequent operations of the method 200B, FIG. 6 further depicts operation 616, which, in one embodiment, processes the data from operations 614 and 615 with an intervention-determining model to output a personalized intervention regimen with adaptive behavioral therapy tools and exercises for improving health and wellbeing of the patient, in relation to his/her specific goals. FIG. 6 also depicts operation 617 where a first module of the intervention regimen is delivered to the patient within the application associated with the system, and operation 618, which provides further adaptation of modules of the intervention regimen as the patient progresses through the intervention regimen and interacts with content.

While the operations of FIG. 6 are shown in a particular order, the operations can be performed in another suitable sequence, omit operations, and/or include additional operations (e.g., based on refinement and training of models, as described below, as well as based on other factors).

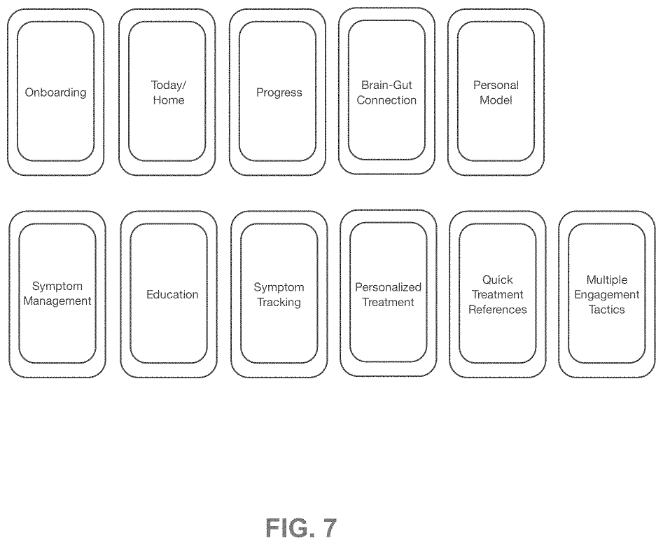

FIG. 7 depicts examples of system aspects of a program for personalized health condition monitoring and improvement, according to one or more embodiments.

As shown in FIG. 7, in one embodiment, components delivered through a PDT system may include content such as, but not limited to: onboarding material, daily (or other time scale) review, progress summaries, brain-gut connection content, personal model analyses, symptom management material, educational material, symptom tracking analyses, personalized treatment analyses, quick references, and multiple engagement tactics material.

Returning now to FIG. 2B, in one embodiment, once a pre-assessment of a patient is performed at operation 226, process flow proceeds to operation 228. In one embodiment, at operation 228, an intervention regimen for the patient is generated upon processing data from the pre-assessment with an intervention-determining model.

In one embodiment, in relation to system components described above, one embodiment of the online system, in coordination with the network and a client device, can process data from the pre-assessment with an intervention-determining model. Operation 228 functions to generate an intervention regimen for the patient upon processing pre-assessment data, in order to design a customized intervention regimen to address specific symptoms and needs of the patient. While operation 228 is described in relation to pre-assessment data, model architecture and associated algorithms can additionally or alternatively be applied to assessment of patient data as the patient interacts with content of the intervention regimen, in order to adaptively modify delivery of intervention regimen components to the patient, with processing of incoming data.

In some embodiments, the intervention-determining model contemporaneously processes data associated with patient goals, user GI and/or inflammatory health condition symptoms, patient mental health states, other characteristics, and interactions with content of the PDT system, providing the intervention regimen as inputs, in order to output a customized and modulatable intervention regimen to improve the health and/or wellbeing of the patient. The intervention-determining model can include architecture for one or more of: conditional decision making (e.g., with conditional branching structure that processes input data in stages and determines an output at each node of the branching structure); ranking (e.g., with ranking algorithms configured to rank candidate intervention regimen components according to appropriateness, based on the input data); matching (e.g., with performance of best match operations between input data and different groups representing modules of the intervention regimen, with centroid-based approaches, etc.); correlation (e.g., correlation functions that process input data to generate outputs associated with different intervention regimen components); and/or any other suitable architecture. Training of models is further described below.

In one embodiment, once an intervention regimen for the patient has been generated at operation 228, method flow proceeds to operation 230. In one embodiment, at 230 the online system, in coordination with other system components (e.g., the client device, external systems, network, etc.) delivers the intervention regimen to the patient, for instance, through an application associated with the PDT system executing at the client device of the patient.

As described in relation to the system above, in various embodiments content associated with the intervention regimen can be of visual (e.g., image format, video format), textual, audio, haptic, and/or other formats, through connected devices (e.g., mobile computing devices, wearable devices, audio output devices, displays, temperature control devices, lighting control devices, etc.) and generated in a manner that promotes user engagement. Furthermore, the system, in providing the interventions (e.g., such as interventions described in more detail below), can coordinate with and/or provide instructions for control of other devices, for intervention delivery. In variations, the system can coordinate with environmental control devices (e.g., connected audio output devices, connected temperature control devices, connected lighting control devices, connected pill dispensing devices, connected smart pill devices, etc.) to change aspects of the patient's environment in association with provision of the intervention regimen.

In one example of an intervention regimen component for reducing anxiety, the intervention regimen can provide a grounding exercise to reduce anxiety regarding GI and/or inflammatory health condition symptoms, where the user is prompted to observe aspects of the environment with multiple senses, and the system can coordinate with environmental control devices to adjust one or more of lighting (e.g., colors, intensity, etc.), sounds (e.g., through audio output devices), and/or temperature in the patient's environment. In another example, the intervention regimen can provide a relaxation exercise to reduce pain associated with GI and/or inflammatory health condition symptoms, and coordinate with an audio output device to play music pleasing to the patient. In another example, the intervention regimen can provide an exercise activity involving movements or dancing, to reduce bloating and depression associated with GI and/or inflammatory health condition symptoms, and coordinate with an audio output device to play dance music to the user, while reducing environmental temperature with a smart thermostat device. The system can provide coordinated interventions, however, in any other suitable manner, where details of interventions are provided in more detail above.

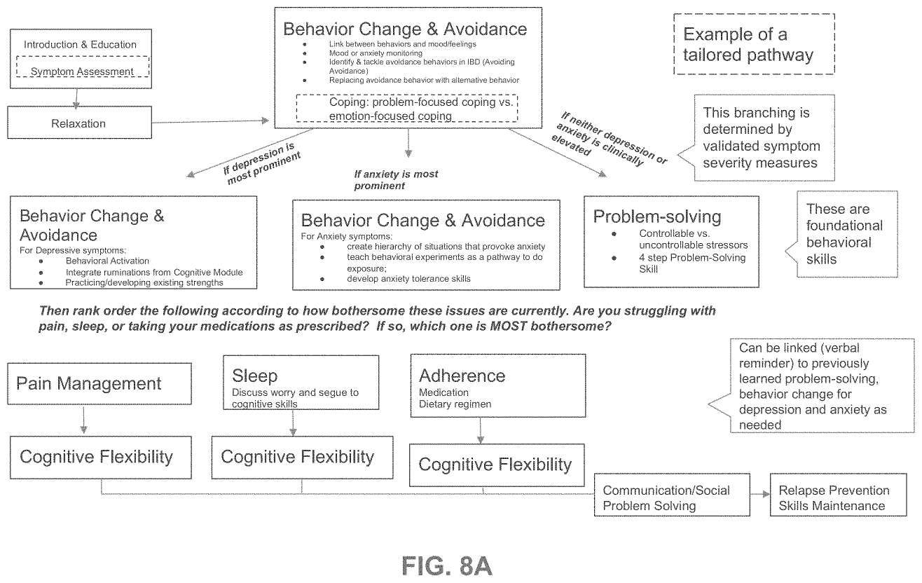

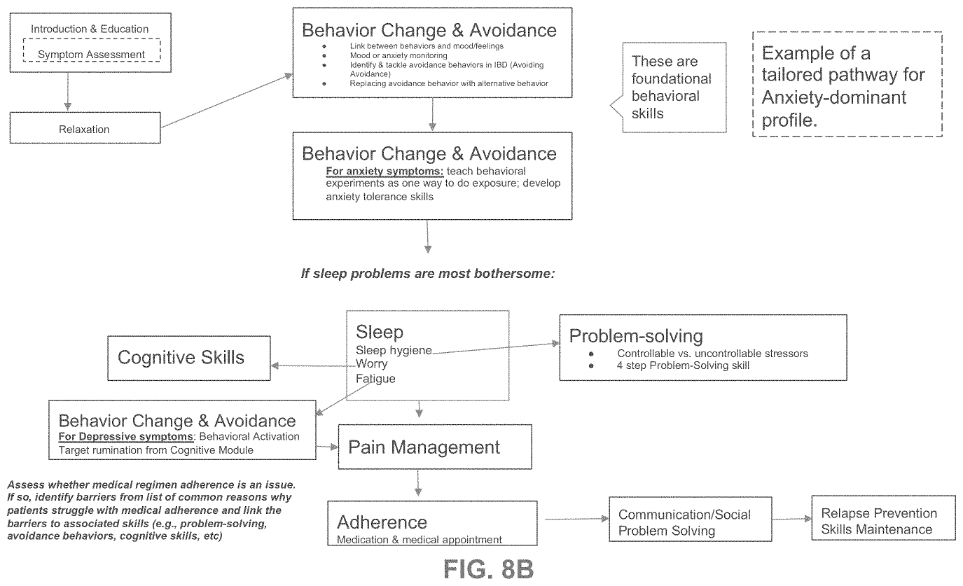

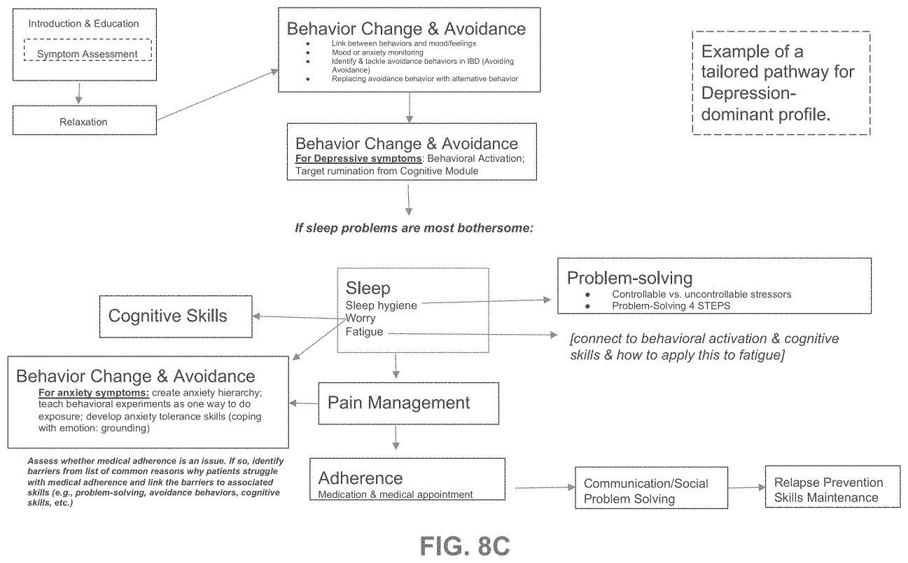

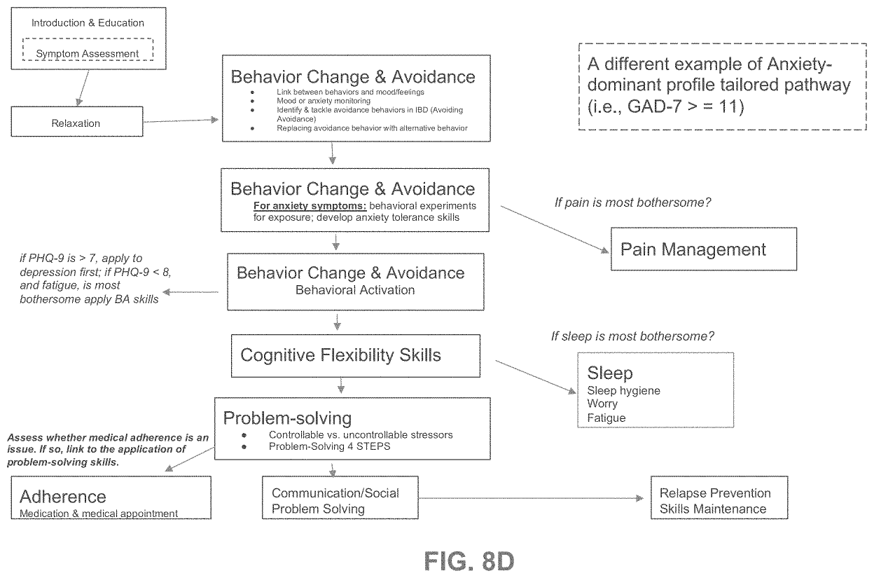

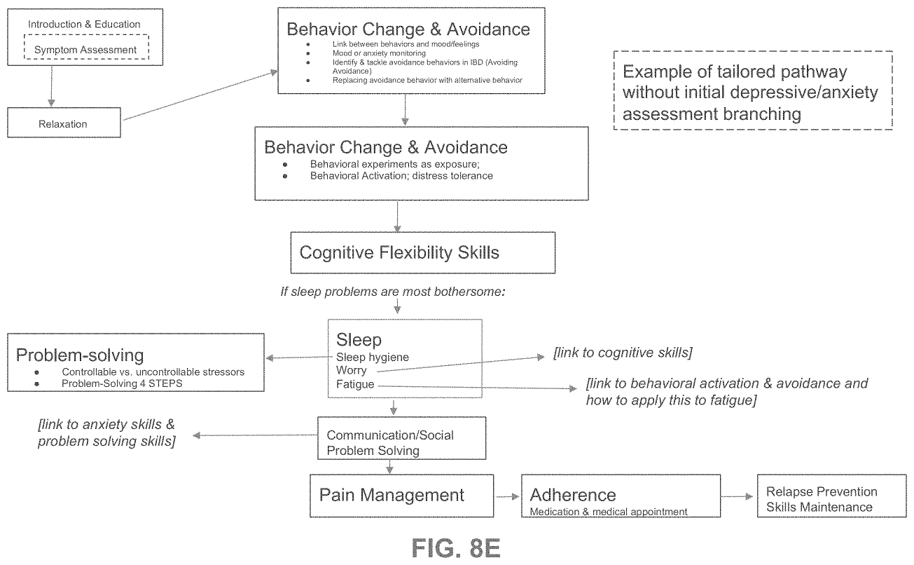

FIG. 8A, FIG. 8B, FIG. 8C, FIG. 8D, and FIG. 8E depict example schematics of conditional branching architecture implemented for delivery of intervention regimen components, according to one or more embodiments.

In various embodiments, as shown in FIG. 8A through FIG. 8E, the intervention-determining model includes architecture for processing input data (e.g., from the pre-assessment and in real-time as the patient interacts with content of the intervention regimen), with a conditional branching model (e.g., with if-then branches coupled to nodes associated with outputs) that processes input data to tailor individual psychological interventions to the patient in an individualized manner. The conditional branching model thus includes decision rules linking characteristics of the patient (e.g., clinical and symptom presentation, demographics, etc.) to different components of the intervention regimen, as an adaptive intervention.

The illustrative embodiment of FIG. 8A depicts architecture of the conditional branching model for a generalized pathway where, based on severity of physical illness symptoms exhibited by a patient, the model guides (e.g., through an application associated with the PDT system executing at the client device) the patient through foundational behavioral skills appropriate to the state and goals of the patient. After a core set of skill building exercises is provided to the patient, the order of modules can vary from patient to patient. Decisions (within app) about which modules to prioritize first are based on patient's presentation and needs (e.g., symptom patterns, etc). For example, if abdominal pain is what is most bothersome to the patient, the digital therapeutic will recommend the pain management module after completing one of the modules (e.g. the relaxation module).

In more detail, based on demonstrated symptoms, in various embodiments the conditional branching model shown in FIG. 8A selects a Behavior Change and Avoidance module for delivery, where the module informs the patient of links between behaviors and moods/feelings, and actively coaches the patient with respect to addressing avoidance behaviors in relation to GI health condition symptoms, in order to replace avoidance behaviors with alternative healthier behaviors. The behavioral therapy techniques implemented in the selected intervention can address problem-focused coping tools and/or emotion-focused coping tools, with additional tailoring for different mental health issues associated with the GI health condition symptoms of the patient. For instance, if the patient's depression is most prominent, the conditional branching model outputs behavioral activation exercises, cognitive reframing techniques, talent practicing and reinforcement exercises, and/or other exercises to mitigate depression symptoms. Additionally or alternatively, if the patient's anxiety is most prominent, the conditional branching model outputs exposure-based exercises associated with anxiety sources, anxiety tolerance skill-building exercises, grounding exercises, and/or other exercises to mitigate anxiety symptoms. Alternatively, if neither anxiety nor depression are elevated, the conditional branching model outputs problem-solving exercises with respect to controllable vs. uncontrollable stressors, and other exercises to mitigate problem-solving issues. The conditional branching model further receives inputs (e.g., rankings of symptom severity) related to symptoms that the patient wishes to improve (e.g., related to pain management, related to sleep, related to adherence, related to communication, related to social problem solving, related to relapse prevention, etc.), and then based upon the inputs, guides the user through additional cognitive skills tailored to improve symptoms in the manner that the patient desires.

The illustrative embodiment of FIG. 8B depicts architecture of the conditional branching model for an anxiety-specific pathway where, based on severity of physical illness symptoms exhibited by a patient, the model guides (e.g., through an application associated with the PDT system executing at the client device) the patient through foundational behavioral skills appropriate to the state and goals of the patient. In more detail, based on demonstrated symptoms, the conditional branching model selects a Behavior Change and Avoidance module for delivery, where the module informs the patient of links between behaviors and moods/feelings, and actively coaches the patient with respect to addressing avoidance behaviors in relation to GI and/or inflammatory health condition symptoms, in order to replace avoidance behaviors with alternative healthier behaviors. For a patient having prominent anxiety symptoms, the conditional branching model of FIG. 8B outputs exposure-based desensitization exercises associated with anxiety sources, anxiety tolerance skill-building exercises, grounding exercises, and/or other exercises to mitigate anxiety symptoms. The conditional branching model of FIG. 8B further receives inputs (e.g., rankings of symptom severity) related to sleep and/or other symptoms (e.g., fatigue, sleep hygiene, worry, etc.) that the patient wishes to improve, and then based upon the inputs, guides the user through additional cognitive skills, problem-solving exercises, and behavior change exercises, tailored to improve sleep symptoms related to his/her GI and/or inflammatory health condition.

The illustrative embodiment of FIG. 8C depicts architecture of the conditional branching model for a depression-specific pathway where, based on severity of physical illness symptoms exhibited by a patient, the model guides (e.g., through an application associated with the PDT system executing at the client device) the patient through foundational behavioral skills appropriate to the state and goals of the patient. In more detail, based on demonstrated symptoms, the conditional branching model selects a Behavior Change and Avoidance module for delivery, where the module informs the patient of links between behaviors and moods/feelings, and actively coaches the patient with respect to addressing avoidance behaviors in relation to GI and/or inflammatory health condition symptoms, in order to replace avoidance behaviors with alternative healthier behaviors. For a patient having prominent depression symptoms, the conditional branching model of FIG. 8C outputs behavioral activation exercises, cognitive reframing techniques, and reinforcement exercises, and/or other exercises to mitigate depression symptoms. The conditional branching model of FIG. 8C further receives inputs (e.g., rankings of symptom severity) related to sleep and/or other symptoms (e.g., fatigue, sleep hygiene, worry, etc.) that the patient wishes to improve, and then based upon the inputs, guides the user through additional cognitive skills, problem-solving exercises, and behavior change exercises, tailored to improve sleep symptoms related to his/her GI and/or inflammatory health condition.

The illustrative embodiment of FIG. 8D depicts architecture of the conditional branching model for a pathway targeted to anxiety and depression (e.g., with a GAD-7 score greater than or equal to 11) where, the model guides (e.g., through an application associated with the PDT system executing at the client device) the patient through foundational behavioral skills appropriate to the state and goals of the patient. In more detail, based on demonstrated symptoms, the conditional branching model selects a Behavior Change and Avoidance module for delivery, where the module informs the patient of links between behaviors and moods/feelings, and actively coaches the patient with respect to addressing avoidance behaviors in relation to GI and/or inflammatory health condition symptoms, in order to replace avoidance behaviors with alternative healthier behaviors. For a patient having prominent anxiety symptoms, the conditional branching model of FIG. 8D outputs exposure-based desensitization exercises associated with anxiety sources, anxiety tolerance skill-building exercises, grounding exercises, and/or other exercises to mitigate anxiety symptoms. The model also determines if the patient is suffering from pain symptoms, and provides the patient with pain management exercises. The model also then sequentially determines if the user is exhibiting symptoms of depression (e.g., if PHQ-9 score is greater than or less than 10), and addresses depression symptoms sequentially relative to other symptoms (e.g., sleep, communication, medication adherence) based upon symptom severity.

The illustrative embodiment of FIG. 8E depicts architecture of the conditional branching model for a pathway that is not specific to anxiety or depression where, based on severity of physical illness symptoms exhibited by a patient, the model guides (e.g., through an application associated with the PDT system executing at the client device) the patient through foundational behavioral skills appropriate to the state and goals of the patient. In more detail, based on demonstrated symptoms, the conditional branching model selects a Behavior Change and Avoidance module for delivery, where the module informs the patient of links between behaviors and moods/feelings, and actively coaches the patient with respect to addressing avoidance behaviors in relation to GI and/or inflammatory health condition symptoms, in order to replace avoidance behaviors with alternative healthier behaviors. For a patient having no initial anxiety/depression symptoms, the conditional branching model of FIG. 8E outputs problem-solving exercises with respect to controllable vs. uncontrollable stressors, and other exercises to mitigate problem-solving issues. The conditional branching model of FIG. 8E further receives inputs (e.g., rankings of symptom severity) related to sleep and/or other symptoms (e.g., fatigue, sleep hygiene, worry, etc.) that the patient wishes to improve, and then based upon the inputs, guides the user through additional cognitive skills, problem-solving exercises, and behavior change exercises, tailored to improve sleep symptoms related to his/her GI and/or inflammatory health condition.

In one embodiment, once the intervention regimen is delivered to the patient at operation 230, method flow proceeds to operation 232. In one embodiment, at operation 232, a set of interactions between the patient and modules of the intervention regimen and a health status progression of the patient are monitored contemporaneously with delivery of the intervention regimen.

In relation to system components described above, an embodiment of the online system, in coordination with the network and a client device, can monitor a set of interactions between the patient and modules of the intervention regimen and a health status progression of the patient contemporaneously with delivery of the intervention regimen. Monitoring interactions functions to provide intimate understanding of progress of the patient in achieving health goals, and to provide further personalization of and delivery of intervention content at appropriate times, in order to maintain or improve progress of the patient. Monitoring is preferably performed in near-real time or real time, such that actions can be taken to adjust interventions to user states according to just-in time adaptive intervention (JITAI) protocols. However, monitoring can be performed with any suitable delay (e.g., in relation to achieving better accuracy of assessed states of the patient).

Monitoring can be performed using survey components delivered with interactive interventions of the intervention regimen, where the user is prompted and provided with interactive elements that allow the patient to provide self-report data indicating progress statuses. Monitoring can additionally or alternatively be performed with processing of other data streams, where the data streams are associated with system or device usage metrics, social networking behavior extracted from usage of social networking platforms and communication platforms, sensor-derived data, and/or other data. Monitoring can thus occur with any frequency and/or level of intrusiveness.

In some embodiments, operation 232 can process monitoring data (e.g., real time data, non-real time data, dynamic data, static data) with a predictive model that outputs indications of one or more of symptom severity predictions, predictions of patient states, indications of predicted success of the patient in achieving goals, and/or other predictions, where training of the predictive model with training sets of data is described in additional detail below.

In one embodiment, ecological momentary assessments of the patient can be used for monitoring. Additionally or alternatively, in one embodiment, client device usage parameters can be used for monitoring. Examples of client device usage parameters can include frequency of application switching, duration of time spent in association with each application login, screen time parameters, data usage associated with different applications and/or types of applications (e.g., social networking, creative, utility, travel, activity-related, etc.) executing on the client device of the patient, time of day of application usage, location of device usage, and other client device usage parameters.

Additionally or alternatively, in one embodiment, the system can process voice data and/or text communication data of the patient for monitoring and modifying interventions and program aspects. Examples of voice data can include voice sampling data from which emotional states can be extracted using voice processing models. In a related manner, natural language processing of textual data (e.g., from communication platforms, from social networking platforms) of the client device can be used to provide context for behaviors of the patient and/or assess emotional or cognitive states of the patient.

Additionally or alternatively, in one embodiment, electronic health record data can be used for monitoring. For instance, if the patient receives medical care, the online system can be configured to receive a notification providing information regarding the type of care the patient has received, and to use this data for monitoring statuses of the patient.

Additionally or alternatively, in one embodiment, the system can include architecture for processing data from other sensors of the client device, devices in the environment of the patient, and/or wearable computing devices can be used for monitoring. Such device data can include activity data, location data, motion data, biometric data, and/or other data configured to provide context to behaviors associated with the health condition of the patient. In one example, motion data from motion of sensors of the client device can indicate that the user is sedentary, and may be experiencing symptoms that can be addressed with components of the intervention regimen. In another example, device usage data can indicate that the patient has been using a particular device (e.g., a tablet device in proximity to the patient, where use does not require extensive motion of the patient), in a fixed location (e.g., from GPS data), and in a prone position (e.g., from motion chip data), and may be experiencing GI health condition symptoms that can be addressed with components of the intervention regimen.

As such, active monitoring of patient states can be used to adjust delivery of intervention regimen modules in order appropriately meet the needs of the patient. Other data and combinations of data can, however, be used for monitoring.

In one embodiment, once a set of interactions between the patient and modules of the intervention regimen and a health status progression of the patient are monitored at operation 232, process flow proceeds to operation 234. In one embodiment at operation 234, in response to at least one of the set of interactions and the health status progression, an action configured to improve wellbeing of the patient with respect to the GI and/or inflammatory health condition is performed.

In relation to system components described above, an embodiment of the online system, in coordination with the network and a client device can, in response to at least one of the set of interactions and the health status progression, perform an action configured to improve health and wellbeing of the patient with respect to the GI and/or inflammatory health condition. Operation 234 functions to provide further customization of the intervention regimen, in order to improve personalization of delivered content to needs of the patient, in an adaptive manner. Operation 234 can also function to increase engagement between the patient and the intervention regimen, in order to improve effectiveness of provided treatments and increase success of the patient in achieving his/her goals.

In various embodiments, the action performed according to operation 234 can include one or more of: adjusting order of and/or content of intervention modules provided, where intervention types and content are described above; updating electronic health records (EHRs), personal health records (PHRs), and/or open medical records, for instance by writing to or modifying records whenever new information is generated regarding the user/patient/patient; providing and/or facilitating provision of supplemental interventions (e.g., hypnotherapy, physical exercises, medications, supplements, etc.) beyond standard content of the intervention regimen, for instance, under physician-guidance or treatment recommendations; generating and/or providing notifications to the patient regarding changes in behavior or health statuses; generating and/or providing notifications to entities (e.g., relatives, acquaintances having permission of the patient, health care providers, etc.) associated with the patient regarding changes in behavior or health statuses; and/or any other suitable action.

In some embodiments, operation 234 can additionally or alternatively include functionality for increasing engagement of the patient with respect to interactions with content of the intervention regimen.

In various embodiments, features for increasing engagement and optimal learning can include text-based functionality for self-monitoring and symptom tracking, where the system can process real time text interactions with provision of interactive tasks, which increases likelihood of patient responses. In more detail, specific descriptions self-reported by the patient can be used in subsequent portions of the intervention regimen to increase personalization of the intervention to drive engagement. Additionally or alternatively, features for increasing engagement and optimal learning can include features that mimic therapist/healthcare provider, or social group interactions (e.g., patient testimonials, clinician video content, etc.). Additionally or alternatively, features for increasing engagement and optimal learning can include features that link the patient's specific current problems (e.g., from operation 232) and/or challenges faced by the patient as a trigger to notify the patient to interact with content of the intervention regimen and recommend appropriate skill for improving health states.

Additionally or alternatively, in some embodiments, engagement can be promoted using one or more of: artificial reality tools (e.g., augmented reality platforms, virtual reality platforms) for reducing depression, anxiety, pain, and/or other symptoms; artificial intelligence-based coaching elements for driving interactions with the patient; smart assistants (e.g., Alexa™ Siri™ Google™ Assistant, etc.) for assisting the patient in relation to task management, gamification elements within intervention regimen-associated applications executing on the client device; gamification elements of other devices (e.g., smart toilet devices having interactive elements, such as buttons that control flushing and other subsystems, for promoting triggering of stool sample tracking in relation to various symptoms); smart pill devices and/or medication-dispensing devices that provide insights in an engaging manner in coordination with intervention regimen modules; adjustment of reinforcement schedules (e.g., in relation to reward sensitivity, positive reinforcement, negative reinforcement, etc.) for providing intervention regimen content to the patient; and other elements for increasing engagement.

As noted above, features for personalization and promoting engagement can be delivered within modules of the intervention regimen before and/or after monitoring of the patient according to operation 232.

In one embodiment, once an action is performed to improve the wellbeing of the patient at operation 234, method flow proceeds to END operation 236, and the method 200B for providing adaptive interventions for gastrointestinal and inflammatory health conditions is exited to await new instructions

### Method 200C

As noted above, FIG. 2C is a flowchart depicting a method 200C for providing adaptive interventions for gastrointestinal and inflammatory health conditions, in accordance with one embodiment.

Referring now to the exemplary embodiment of FIG. 2C, in one embodiment, method 200C begins at BEGIN 238, and method flow proceeds to operation 240. In one embodiment, at operation 240, an interface between a device and a user is established.

In one embodiment, once an interface between a device and a user is established at operation 240, method flow proceeds to operation 242. In one embodiment, at operation 242, a set of signals associated with a gastrointestinal (GI) and/or inflammatory health condition of the user is received from the interface, wherein the set of signals encodes physiological data, behavioral data, environmental stress data, emotional data, and cognitive data of the user.

In one embodiment, once a set of signals associated with a gastrointestinal (GI) and/or inflammatory health condition of the user is received from the interface at operation 242, method flow proceeds to operation 244. In one embodiment, at operation 244, a characterization of the GI and/or inflammatory health condition is determined upon processing the set of signals with a model.

In one embodiment, once a characterization of the GI and/or inflammatory health condition is determined at operation 2244, method flow proceeds to operation 246. In one embodiment, at operation 246, based upon the characterization, content of a treatment comprising a set of components is modulated, wherein the set of components comprises a subset of cognitive behavioral therapy (CBT) components for improving a state of the user.

In one embodiment, once content of a treatment is modulated at operation 246, method flow proceeds to operation 248. In one embodiment, at operation 248, the treatment is administered to the user.

In one embodiment, once the treatment is administered to the user at operation 248, method flow proceeds to END operation 250, and the method 200C for providing adaptive interventions for gastrointestinal and inflammatory health conditions is exited to await new instructions

### Additional Method Aspects

As noted above, in various embodiments, the methods 200A, 200B, and/or 200C can further include operations for detecting performance of activities associated with the intervention regimen, by the patient; reinforcing user performance or engagement with the intervention regimen; determining undesired levels of performance or engagement with the intervention regimen; and driving improved engagement with the intervention. For instance, in relation to various activities of the intervention regimen, in one embodiment, the methods 200A, 200B, and/or 200C can include functionality for detecting performance or non-performance of activities (e.g., based on system engagement, based upon sensor-detected measures of activity, etc.). If the patient performs activities of the intervention regimen appropriately, the methods 200A, 200B, and/or 200C can include functionality for reinforcing performance through provision of various rewards (e.g., rests, rewards of monetary value, etc.). If the patient does not perform activities appropriately, the methods 200A, 200B, and/or 200C can include functionality for determining causes of non-performance (e.g., non-engaging content, external factors associated with the patient's life, etc.) and adjust content delivery, provide modified interventions, and/or adjust reinforcement schedules accordingly.

Furthermore, as indicated above, in various embodiments, the methods 200A, 200B, and/or 200C can include functionality for developing and training predictive models for predicting states of the patient during the course of the intervention regimen, in order to improve chances of success in outcomes. The methods 200A, 200B, and/or 200C can thus include functionality for aggregation of training datasets from various data sources described above, and processing training datasets with one or more types of model architecture in order to improve predictions and/or selection of appropriate modules of the intervention regimen for delivery to the patient. Models associated with the methods 200A, 200B, and/or 200C can be defined within architecture of computing systems described above, and include elements for statistical analysis of data and/or machine learning.

In some embodiments, input to a machine learning module comprises one or more textual words, phrases, or lengthier strings. In some embodiments, the input comprises various data elements, such as numerical values corresponding to user responses to a series of questions in a questionnaire (e.g., ranking various symptom severities on a scale). In some embodiments, one or more output values of a machine learning module comprise values representing a classification of a particular condition of a user. In some embodiments, machine learning modules implementing machine learning techniques are trained, for example using curated and/or manually annotated datasets. Such training may be used to determine various parameters of machine learning algorithms implemented by a machine learning module, such as weights associated with layers in neural networks. In some embodiments, once a machine learning module is trained, e.g., to accomplish a specific task such as classifying a condition subtype, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, for example to dynamically update the machine learning module. In some embodiments, a trained machine learning module is a classification algorithm with adjustable and/or fixed (e.g., locked) parameters, e.g., a random forest classifier. In some embodiments, two or more machine learning modules may be combined and implemented as a single module and/or a single software application. In some embodiments, two or more machine learning modules may also be implemented separately, e.g., as separate modules or applications. A machine learning module may be software and/or hardware. For example, a machine learning module may be implemented entirely as software, or certain functions of a ANN module may be carried out via specialized hardware (e.g., via an application specific integrated circuit (ASIC)).

In more detail related to model training, in various embodiments, the method can include: generating a combined dataset upon applying a first set of transformations to an aggregate dataset including physiological data, behavioral data, environmental stress data, emotional data, and cognitive data from a set of users exhibiting a form of the GI and/or inflammatory health condition; collecting a treatment dataset comprising treatment outcome labels (e.g., quantitative or qualitative labels describing efficacy of individual treatment components) associated with the subset of behavioral therapy components applied to the set of users; creating a first training dataset comprising the combined dataset and the treatment dataset; and training the model with the first training dataset. As such, the model can be structured and ultimately refined for receiving data objects associated with at least one of: physiological data, behavioral data, environmental stress data, emotional data, and cognitive data of the user, and returning a set of outputs comprising a selection of treatment subcomponents tagged with efficacy indicators.

Statistical analyses and/or machine learning algorithm(s) can be characterized by a learning style including any one or more of: supervised learning (e.g., using back propagation neural networks), unsupervised learning (e.g., K-means clustering), semi-supervised learning, reinforcement learning (e.g., using a Q-learning algorithm, using temporal difference learning, etc.), and any other suitable learning style.

Furthermore, any algorithm(s) can implement any one or more of: a regression algorithm, an instance-based method (e.g., k-nearest neighbor, learning vector quantization, self-organizing map, etc.), a regularization method, a decision tree learning method (e.g., classification and regression tree, chi-squared approach, random forest approach, multivariate adaptive approach, gradient boosting machine approach, etc.), a Bayesian method (e.g., naïve Bayes, Bayesian belief network, etc.), a kernel method (e.g., a support vector machine, a linear discriminate analysis, etc.), a clustering method (e.g., k-means clustering), an associated rule learning algorithm (e.g., an Apriori algorithm), an artificial neural network model (e.g., a back-propagation method, a Hopfield network method, a learning vector quantization method, etc.), a deep learning algorithm (e.g., a Boltzmann machine, a convolution network method, a stacked auto-encoder method, etc.), a dimensionality reduction method (e.g., principal component analysis, partial least squares regression, etc.), an ensemble method (e.g., boosting, boot strapped aggregation, gradient boosting machine approach, etc.), and any suitable form of algorithm.

### User Interface

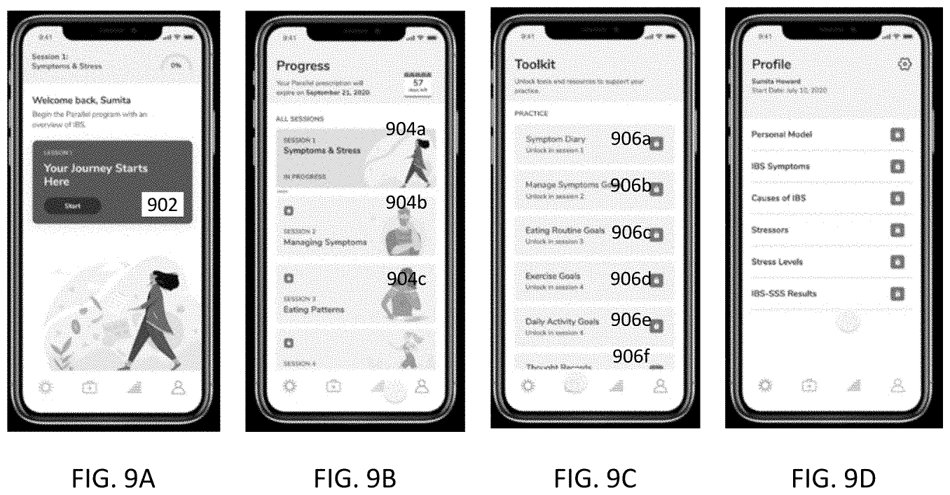

FIG. 9A, FIG. 9B, FIG. 9C, and FIG. 9D are screenshots of several portions of an exemplary GUI for a system for treating gastrointestinal and/or inflammatory health conditions using digital therapeutics, according to one or more embodiments. Screenshots in FIG. 9A, FIG. 9B, FIG. 9C, and FIG. 9D show a GUI in an initial state, corresponding to a starting point for a user that has just begun to use the system.

FIG. 9A shows a home screen that provides user access to an initial, first, lesson module. As shown in the figure, a selectable icon 902 representing the initial lesson module may be displayed, which a user may select to begin.

FIG. 9B shows another screen of a GUI, through which a user may track their progress through a sequence of interactive lesson modules. In the example shown, lesson modules are grouped into sub-sequences referred to as sessions, with each session comprising lessons that are related, e.g., thematically. For example, FIG. 9B shows a first icon 904a representing a first session directed to symptoms and stress, a second icon 904b representing a second session comprising lessons that introduce a user to tools for managing symptoms, and a third icon 904c representing a third session comprising lessons related to managing eating patterns. As shown in FIG. 9B, in some embodiments, a GUI restricts user access to subsequent sessions (i.e., sessions two and above in the example of FIG. 9B, which fall after session one) until the user has completed the first session. A GUI may include visual cues indicating which sessions are accessible and which are locked including, for example, graphical indicators (e.g., a picture of a lock), shading, colorization, etc., as shown in FIG. 9B.

Turning to FIG. 9C, in some embodiments, certain lesson modules are associated with practice modules that allow a user to use and practice particular skills on a regular and/or as needed basis, outside of a sequence of lesson modules. In some embodiments, access to a particular practice module is restricted until the user has completed a lesson module with which it is associated. For example, as shown in FIG. 9C, practice modules indicated via icons 906a, 906b, 906c, 906d, 906e, and 906f are initially locked, but may be unlocked via completion of various associated lessons. In some embodiments, lesson modules may also be associated with resources, such as, but not limited to, video guides to particular breathing techniques and/or relevant articles. As with practice modules, access to resources may also be restricted such that a particular resource is locked (e.g., inaccessible) until a lesson module with which it is associated has been completed.

Turning to FIG. 9D, a GUI may also comprise a stored profile of the user. A user profile may be populated via various lesson modules that solicit input from the user, for example regarding personal characteristics, thoughts and feelings, symptom logging, identification of stressors, stress level tracking, and completion of diagnostic assessments aimed at characterizing their condition. In some embodiments, various patient reported outcome instruments, which, for example, measure condition symptom severity, quality of life, etc., can be used. As one illustrative example, in some embodiments, a patient/user suffering from IBS symptoms may complete an IBS symptom severity scale (IBS-SSS) evaluation, and/or other evaluations based on other patient reported outcome instruments, described in further detail above.

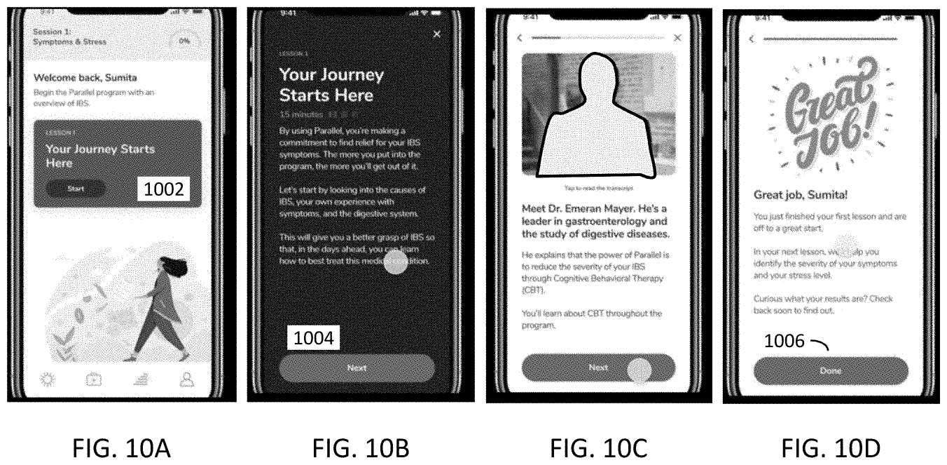

FIG. 10A, FIG. 10B, FIG. 10C, and FIG. 10D are screenshots of example user interactions with an initial lesson module for content tailored for a patient with IBS, according to one or more embodiments.

As shown in FIG. 10A, a user may select icon 1002 representing the initial lesson module from their home-screen to begin the initial lesson module. As shown in FIG. 10B and FIG. 10C, in some embodiments, a user may step through content of a lesson module by tapping a selectable graphical button 1004, until they have viewed all screens comprising all graphical content and widgets of the lesson module. As shown in FIG. 10D, in some embodiments, a user may be presented with a final screen that provides an indication that they have completed a particular lesson module. The user may select a graphical button 1006 to confirm completion of the particular lesson module. Following completion of an initial lesson module, the user may progress onto a subsequent one.

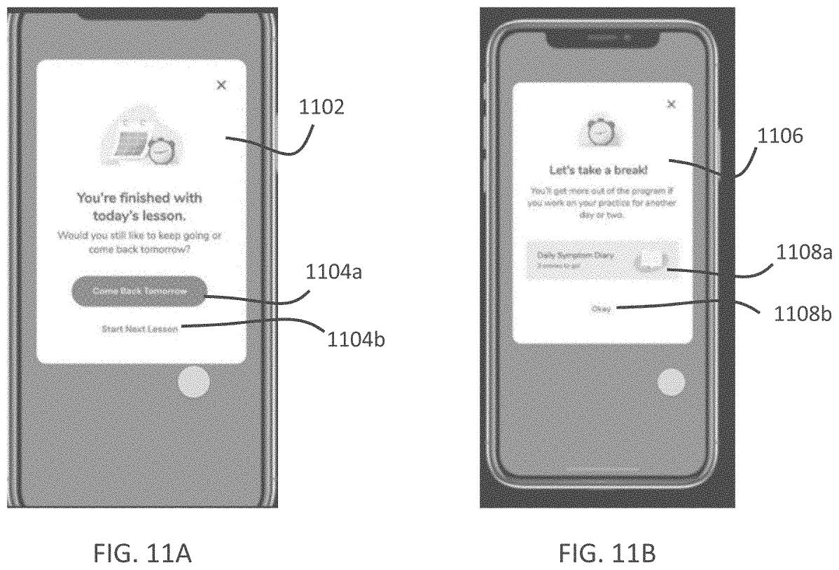

FIG. 11A and FIG. 11B are screenshots showing gate features of an exemplary GUI for a system for treating gastrointestinal and/or inflammatory health conditions using digital therapeutics, in accordance with one or more embodiments.

In some embodiments, progression onto a next lesson module is not necessarily instantaneous and/or direct. Instead, as described above, gate features may be used to introduce friction and/or to control a rate of progression from one lesson module to a next. FIG. 11A shows an example soft-gate, wherein a user is not fully prevented from beginning a second lesson module, but is encouraged to delay moving on, and required to provide additional input to do so. Inter-lesson gate screen 1102 represents a soft-gate, and includes graphical content prompting the user to delay progressing onto the second lesson until the next day. Example screen 1104 also includes delay button 1104a and continue button 1104b graphical widgets, wherein selection of the delay button 1104a graphical widget returns the user to the home-screen. The user may, if they desire, progress to the second lesson module by selecting the continue button 1104b graphical widget. As shown in FIG. 11A, in some embodiments, delay button 1104a and continue button 1104b graphical widgets are rendered graphically so as to visually emphasize delay button 1104a, and de-emphasize continue button 1104b, thereby encouraging the user to delay moving on to the second lesson module.

As described herein, evaluation of various criteria may be used as control gate features. In the example shown in FIG. 11A, a soft-gate is based on time-relationship criteria with respect to a user's time of completion of the first lesson module. In particular, in the example of FIG. 11A, a soft-gate is used to encourage a user to wait until a next day to begin the second lesson module.

Turning to FIG. 11B, in some embodiments, gates may be used to encourage user practice of particular behavioral skills. For example, as shown in FIG. 11B, inter-lesson gate screen 1106 represents a hard-gate and does not allow for continued user progression onto the second lesson module. Instead, inter-lesson gate screen 1106 includes graphical content that encourages the user to take a break and practice particular behavioral skills via a practice module identified by graphical icon 506a. In some embodiments, graphical icon 1108a is a graphical widget—e.g., a selectable graphical button. Accordingly, in some embodiments, a graphical icon 1108a provides a link to a particular practice module, such that user selection of the graphical icon 1108a causes initiation (e.g., display) of a particular practice module to which it links (e.g., a symptom diary practice module, as shown in the example screen of FIG. 11B, or other practice modules). In some embodiments, for example as shown in FIG. 11B, an inter-lesson gate screen may comprise a graphical widget that returns a user to a home screen. For example, as shown in FIG. 11B, inter-lesson gate screen 1106 comprises graphical widget 1108b, displaying text “Okay,” whereupon a user selection of graphical widget 1108b, they are returned to a home screen shown.

It should be noted here that the above described screenshots of several portions of an example GUI for a system providing guided behavioral therapy are given for illustrative purposes only and are not intended to limit the scope of the invention as claimed below and as disclosed herein.

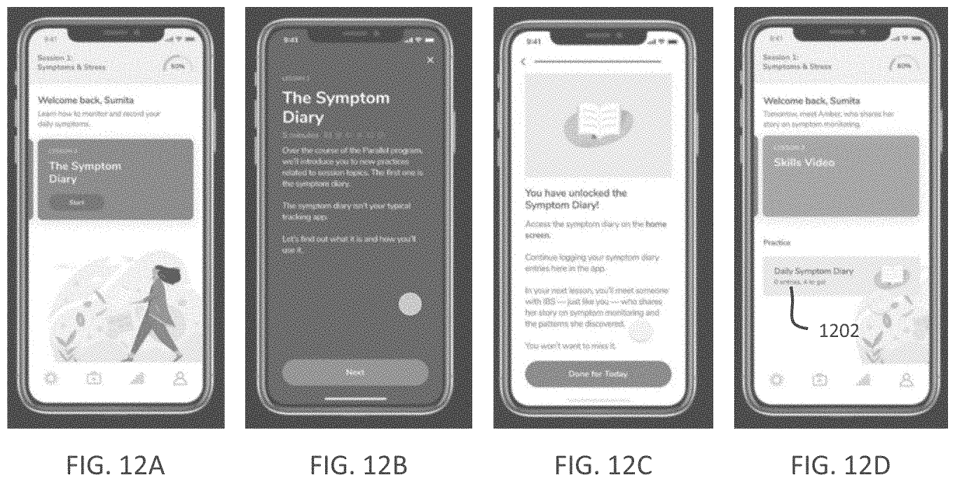

FIG. 12A, FIG. 12B, FIG. 12C, and FIG. 12D are screenshots of an exemplary GUI for a symptom diary lesson module, according to one or more embodiments.

In some embodiments, a sequence of interactive lesson modules may include a symptom diary lesson module that introduces and familiarizes a user with techniques for tracking their GI and/or inflammatory health condition symptoms. In particular, in some embodiments, technologies described herein provide a convenient GUI that can (e.g., be demonstrated and/or designed to) facilitate user tracking and/or monitoring of their GI and/or inflammatory health symptoms on a regular basis. In some embodiments, such a tracking GUI may be included within a same system that provides, and controls user progression through interactive lesson modules, for example as a symptom diary practice module associated with a symptom diary lesson module. In some embodiments, access to a symptom diary practice module may be unlocked following completion of a symptom diary lesson module by the user, for example, as shown in FIG. 12C. As shown in FIG. 12D, once the user has unlocked the symptom diary practice module, a selectable icon 1202 representing, and providing access to, the symptom diary practice module may be displayed on a user home-screen.

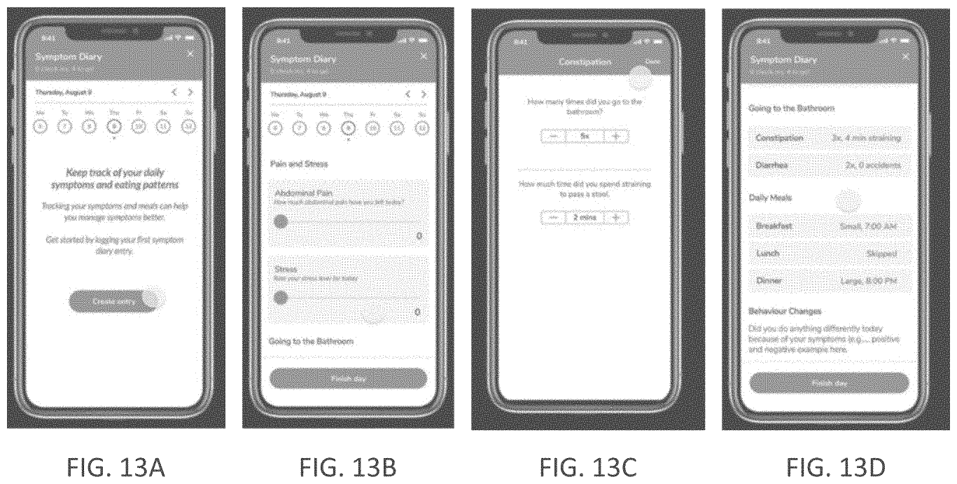

FIG. 13A, FIG. 13B, FIG. 13C, and FIG. 13D are screenshots of example user interactions with a symptom diary practice module, in accordance with one or more embodiments. In various embodiments, a symptom diary practice module may be a stand-alone module, or it may be part of a different module, such as, but not limited to, the introduction and education module discussed above.

In various embodiments, for each day, a user may use graphical widgets to provide input associated with one or more aspects of their disease, disorder, and/or condition. Examples of patient input include, but are not limited to, rating pain and stress on a scale (FIG. 13B), providing ratings characterizing additional symptoms specific to their disease, disorder, and/or condition (FIG. 13C) and providing information characterizing their daily meals (FIG. 13D).

It should be noted again here that the above described screenshots of several portions of an example GUI for a system providing guided behavioral therapy are given for illustrative purposes only and are not intended to limit the scope of the invention as claimed below and as disclosed herein.

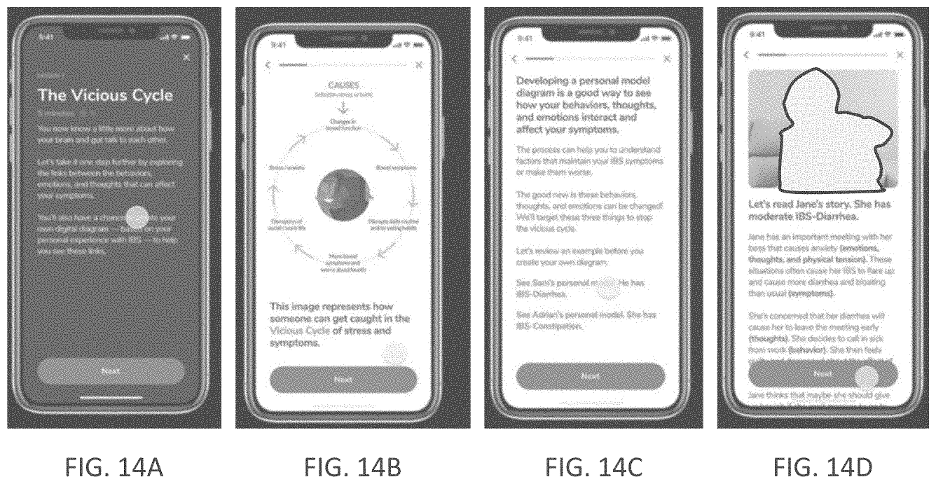

FIG. 14A, FIG. 14B, FIG. 14C, and FIG. 14D are screenshots of an exemplary GUI for introducing a personal model lesson module, in accordance with one or more embodiments. In various embodiments, a personal model lesson module may be a stand-alone module, or it may be part of a different module, such as, but not limited to, the physical illness narrative module discussed above.

As noted above, in some embodiments a sequence of interactive lesson modules includes a personal model lesson module that allows a user to identify cycles of behaviors, thoughts, emotions, and stressors that influence symptoms associated with their particular condition (e.g., from which they are suffering). In some embodiments, a personal model lesson module is used to implement, via a GUI, a structured process for conveniently soliciting user input of specific counter-productive behaviors, unhelpful thoughts, and negative emotions that they identify, e.g., in their life and/or as associated with their particular condition. In some embodiments, a particular condition from which the user is suffering is a GI and/or inflammatory health condition, such as, but not limited to, IBS.

In some embodiments, prior to soliciting user input and creating a user's own personal model, a personal model lesson module introduces a user to process for creating a personal model, for example so as to orient them and provide content designed to offer helpful motivation.

As shown in FIG. 14A, FIG. 14B, and FIG. 14C, in some embodiments, graphical content representing educational material is displayed to a user, for example to introduce them to concept of vicious cycles, and explain how symptoms, stress, and pain can create a feedback loop.

In some embodiments, graphical content corresponding to shared user experiences and/or testimonials, e.g., in video, written, audio, etc. format, is displayed. For example, as shown in FIG. 14D, a user may be prompted to read about another user's experiences with their GI and/or inflammatory health condition and guided behavioral therapy approaches such as those described herein. In some embodiments, a user may view exemplary personal models created by and shared by others. Other lesson modules, for example any lesson modules described herein and/or additional lesson modules, providing for development of other behavioral therapy skills provided via the technologies described herein may also include content comprising patient experiences and/or testimonials.

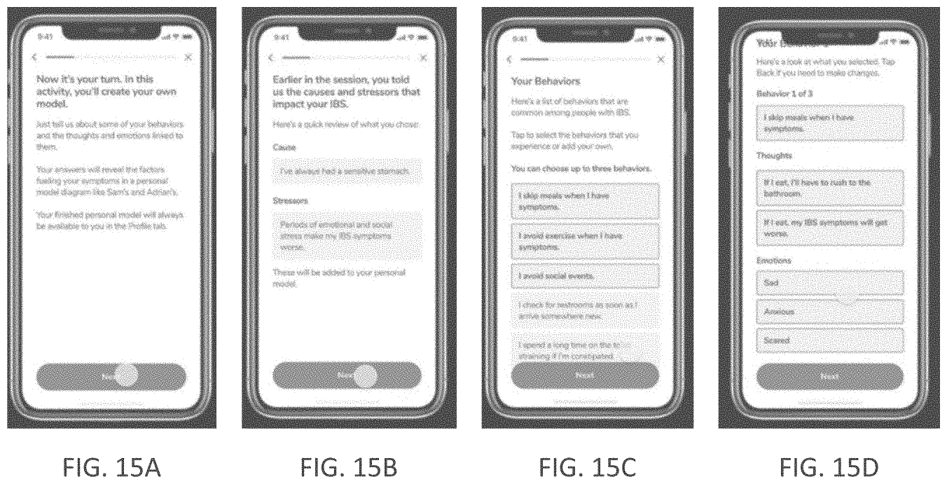

FIG. 15A, FIG. 15B, FIG. 15C, and FIG. 15D are screenshots of an exemplary GUI for a personal model lesson module, according to one or more embodiments. In various embodiments, a personal model lesson module may be a stand-alone module, or it may be part of a different module, such as, but not limited to, the physical illness narrative module discussed above.

In some embodiments, a personal model lesson module may retrieve stored information, previously input by a user. For example, in various embodiments, a user may have previously provided input identifying causes and/or stressors that impact their particular GI and/or inflammatory health condition, and previously input user identifications of causes and stressors that impact the user's GI and/or inflammatory health condition may be retrieved and displayed, as shown in FIG. 15B. In some embodiments, a user provides input corresponding to causes and/or stressors associated with their particular GI and/or inflammatory health condition via a personal model lesson module. In some embodiments, a user may be provided with a graphical list of selectable elements. In some embodiments, the user is prompted to select a pre-defined number of counter-productive behaviors, as shown in FIG. 15C. As shown in FIG. 15D, in some embodiments, for each user selected counter-productive behavior, the user is prompted to select one or more unhelpful thoughts related to the counter-productive behavior. In some embodiments, unhelpful thoughts are selected from a list of pre-defined thoughts. In some embodiments, a user may provide free-form textual input, for example via a text box. In some embodiments, following a user selection of one or more unhelpful thoughts, a user is prompted to select one or more negative emotions. In some embodiments, negative emotions are selected from a list of pre-defined emotions. In some embodiments, a user may provide free-form textual input, for example via a text box.

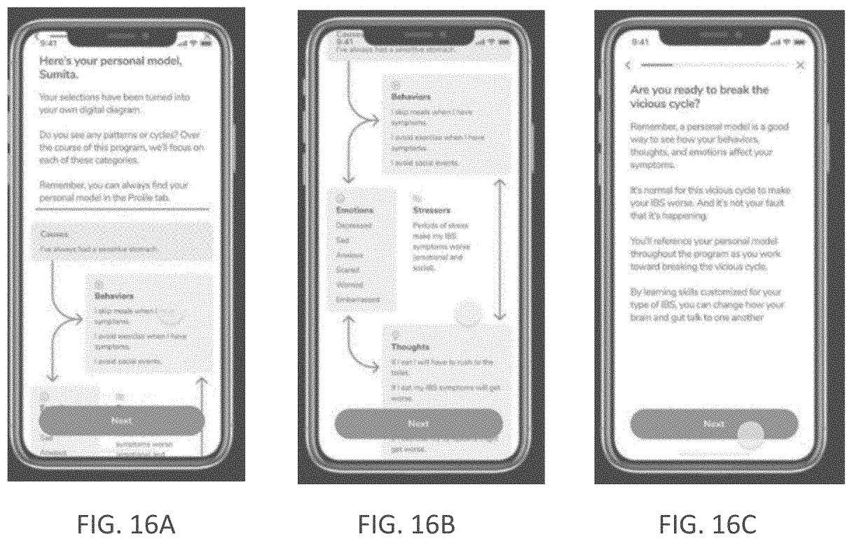

FIG. FIGS. 16A, 16B, and 16C are screenshots of an exemplary personal model graphical representation, according to one or more embodiments.

In some embodiments, once a user has completed entry of counter-productive behaviors, unhelpful thoughts, and negative emotions, received user input is used to create a personal model graphical representation, which can be displayed on a user computing device. In some embodiments, a personal model graphical representation comprises text corresponding to user selected counter-productive behavior(s), unhelpful thought(s), and negative emotion(s), superimposed on a flow diagram illustrating links between each other, as shown in FIG. 16A and FIG. 16B. In some embodiments, a personal model graphical representation comprises text corresponding to causes and/or stressors of symptoms, previously input by the user and retrieved via the personal model lesson module and/or input within the personal model lesson module, as described herein.

In some embodiments, a personal model graphical representation is rendered to have a form factor fitting one or more mobile device screens. For example, as shown in FIGS. 16A and 16B, in some embodiments, a personal model graphical representation can be rendered in a narrow, rectangular format, allowing a user to scroll through the rendered diagram to view its content. In some embodiments, a personal model graphical representation can be displayed as a zoom-able diagram, such that a user may zoom in and out to view portions of the diagram. In some embodiments, a personal model graphical representation can be displayed so as to allow a user to navigate through its content by panning, for example in a two-dimensional fashion.

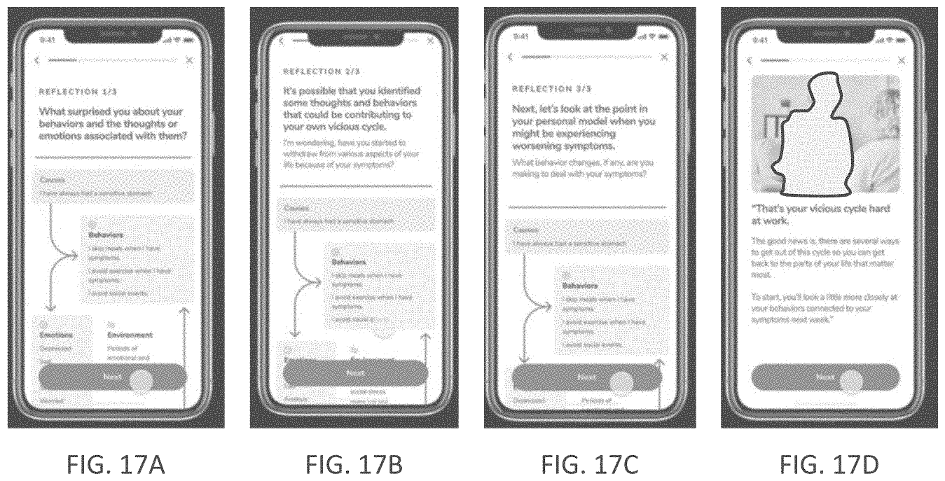

FIG. 17A, FIG. 17B, FIG. 17C, and FIG. 17D are screenshots of an exemplary GUI for a reflections section of a personal model lesson module, according to one or more embodiments.

In some embodiments, a personal model lesson module includes graphical content prompting a user to review their personal model. In particular, in some embodiments, a series of questions (e.g., from a predefined list of questions, e.g., based on a therapeutic protocol) are displayed and presented to the user along with their personal model graphical representation, prompting the user to consider their selections, identify links, consider possible changes in their behavior that could be implemented to address their symptoms, and the like. For example, in FIG. 17A, FIG. 17B, and FIG. 17C, a sequence of user questions are presented. Graphical content, including passages of rendered text, mimicking conversation with a therapist can be displayed. In some embodiments, encouraging graphical content is displayed, and the user is returned to a home screen.

It should be noted again here that the above described screenshots of several portions of an example GUI for a system providing guided behavioral therapy are given for illustrative purposes only and are not intended to limit the scope of the invention as claimed below and as disclosed herein.

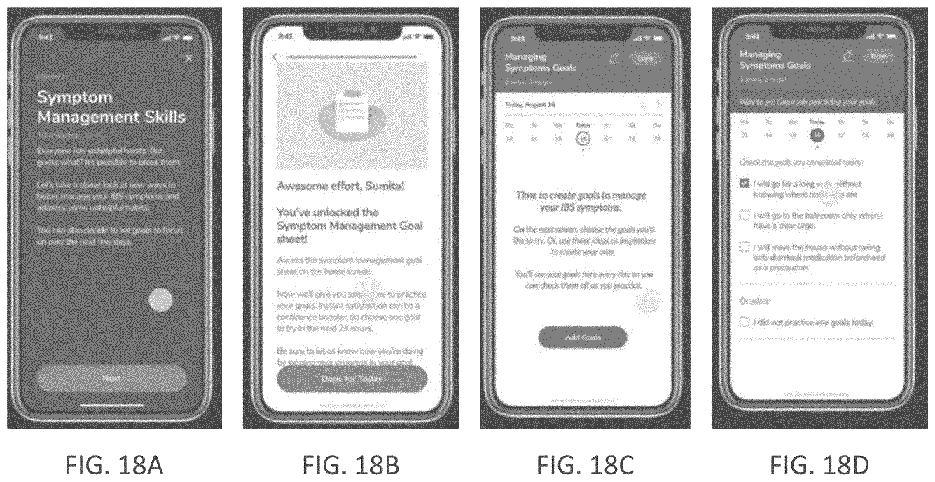

FIG. 18A, FIG. 18B, FIG. 18C, and FIG. 18D are screenshots of an exemplary GUI for a symptom management lesson module, according to one or more embodiments. In various embodiments, a symptom management lesson module may be a stand-alone module, or it may be part of a different module, such as, but not limited to, the pain management module discussed above.

FIG. 18A, FIG. 18B, FIG. 18C, and FIG. 18D show an example symptom management lesson module and an associated symptom management goals practice module. In one embodiment, upon user completion of a symptom management lesson module, as shown in FIG. 18A, a symptom management goals practice module can be unlocked and made accessible to the user, as shown in FIG. 18B. As shown in FIG. 18C and FIG. 18D, a user may access a symptom management goals module to create goals to manage their GI and/or inflammatory health condition symptoms. A user may, e.g., regularly, use a goals module to set goals such as goals pertaining to a regular timing and/or type of food and/or activity (e.g., physical exercise, meditation, breathing and/or relaxation exercises, reflection exercise, e.g., including skills introduced via lesson modules and/or practicable via interaction with one or more other practice modules, such as keeping a regular symptom diary, etc.). In some embodiments, for example as described in further detail herein, a goals module may provide for setting goals pertaining to medication adherence.

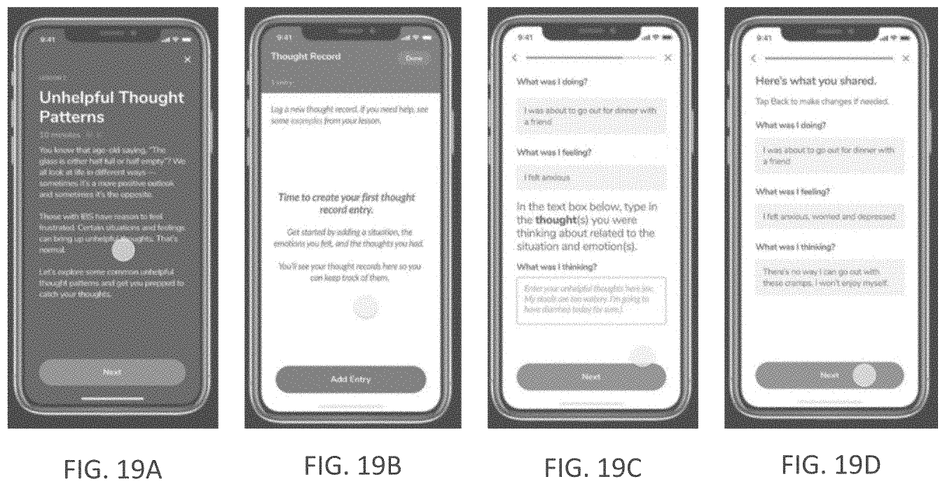

FIG. 19A, FIG. 19B, FIG. 19C, and FIG. 19D are screenshots of an exemplary GUI for a unhelpful thought pattern lesson module, according to one or more embodiments. In various embodiments, an unhelpful thought pattern lesson module may be a stand-alone module, or it may be part of a different module, such as, but not limited to, the cognitive restructuring and flexibility module discussed above.

FIG. 19A, FIG. 19B, FIG. 19C, and FIG. 19D show an example unhelpful thought pattern lesson module and an associated thought record practice module. In one embodiment, upon user completion of an unhelpful thought pattern lesson module, as shown in FIG. 19A, a thought record practice module can be unlocked and made accessible to the user. As shown in FIG. 19B, FIG. 19C, and FIG. 19D, a user may access a thought record practice module to create thought entries tracking their thoughts and associated activities and feelings.

In some embodiments, following completion of various lesson modules and sessions comprising lesson module sequences, completed sessions may be indicated visually to a user. In some embodiments, completion of certain lesson modules unlocks various associated practice modules, which are then made accessible to the user via a screen of a GUI. In some embodiments, completion of lesson modules may cause population of portions of a user profile, which a user may review via a GUI. For example, in one embodiment, completion of a user personal model module as described herein provides for creation of a personal model that identifies a particular user's individual vicious cycle of related stressors, behaviors, emotions, and thoughts. In some embodiments, the data corresponding to a previously created personal model is stored and may be rendered for review and reflection by a user, via a profile screen.

In some embodiments, interactive lesson modules, associated practice modules, user profile content, and the like, may include a variety of other lesson modules, additionally or alternatively to those described herein. In some embodiments, various lesson modules and content thereof as described herein may be combined with other content, for example from other lesson modules described herein.

### CONCLUSION

As noted above, embodiments of the present disclosure provide a technical solution to the technical problem of effectively, efficiently, and remotely treating gastrointestinal and inflammatory health conditions using prescription digital therapeutics in order to ensure that patients receive adequate care, support, and treatment for their GI and/or inflammatory health condition. The inventions covered by the system and method disclosed herein can confer several benefits over conventional systems and methods, and such inventions are further implemented into many practical applications related to improvement of user health.

In one embodiment, a computing system implemented method for treating gastrointestinal and inflammatory health conditions using prescription digital therapeutics comprises: providing a patient with a user interface to a therapeutics system; performing, by the therapeutics system, a pre-assessment of a patient exhibiting one or more health condition symptoms; generating, by the therapeutics system, patient profile and pre-assessment data based on the results of the patient pre-assessment; processing, by the therapeutics system, the patient profile and pre-assessment data to generate patient condition data; processing, by the therapeutics system, the patient profile and pre-assessment data and the patient condition data to generate a personalized intervention regimen for the patient wherein the personalized intervention regimen for the patient defines one or more interactive therapy modules to be administered to the patient;

In one embodiment, a computing system implemented method for treating gastrointestinal and inflammatory health conditions using prescription digital therapeutics further comprises: administering the one or more interactive therapy modules to the patient through the user interface of the therapeutics system according to the personalized intervention regimen generated for the patient; monitoring the patient's interactions with the content of the one or more interactive therapy modules to generate patient interaction data representing the patient's interactions with the content of the one or more interactive therapy modules; at least partly based on the patient interaction data, dynamically controlling patient progression through the one or more interactive therapy modules; at least partly based on the patient interaction data, dynamically updating the patient's personalized intervention regimen; and administering one or more interactive therapy modules to the patient through the user interface of the therapeutics system according to the updated personalized intervention regimen generated for the patient.

In one embodiment, the therapeutics system is a prescription digital therapeutics (PDT) system. In one embodiment, the patient health condition symptoms are associated with one or more of: a gastrointestinal (GI) health condition; and an inflammatory health condition.

In one embodiment, performing a pre-assessment of a patient includes utilizing surveying tools derived from one or more patient reported outcome instruments selected from the group of patient reported outcome instruments consisting of: a Functional Bowel Disorder Severity Index (FBDSI) instrument; a Gastrointestinal Symptom Rating Scale-IBS (GSRS-IBS) instrument; an IBS-Adequate Relief (IBS-AR) instrument; an IBS Global Assessment of Improvement Scale (IBS-GAI) instrument; an IBS Symptom Severity Score (IBS-SSS) instrument; an IBS Quality of Life (IBS-QOL) instrument; a Patient Reported Outcome Measurement Information System (PROMIS) instrument; a Visual Analogue Scale for Irritable Bowel Syndrome (VAS-IBS); a work and social adjustment scale (WSAS)-derived instrument; a clinical disease activity measurement instrument; a PHQ-9 patient health questionnaire; a GAD-7 anxiety disorder questionnaire; and a pain assessment questionnaire.

In one embodiment, performing a pre-assessment of a patient includes utilizing non-survey data obtained from one or more devices selected from the group of devices consisting of: electronic health record-associated devices; torso-coupled devices; wearable devices; ingestible devices; implanted devices; smart-pill devices; smart toilet devices; location monitoring devices; and social networking tracking devices.

In one embodiment, generating the patient condition data includes of one or more of: identifying the patient condition as a gastrointestinal (GI) health condition; identifying a subtype of the gastrointestinal (GI) health condition; identifying a severity of the gastrointestinal (GI) health condition; identifying the patient condition as an inflammatory health condition; identifying a subtype of the inflammatory health condition; and identifying a severity of the inflammatory health condition.

In one embodiment, the one or more interactive therapy modules administered to the patient according to the personalized intervention regimen for the patient are part of a guided behavioral therapy treatment, further wherein the guided behavioral therapy treatment is administered remotely through a user interface of the therapeutics system. In one embodiment, the one or more interactive therapy modules of the guided behavioral therapy treatment include components of therapies selected from the group of therapies consisting of: psychotherapy; cognitive behavioral therapy (CBT); acceptance commitment therapy (ACT); dialectical behavioral therapy (DBT); exposure therapy; mindfulness-based cognitive therapy (MCBT); hypnotherapy; experiential therapy; and psychodynamic therapy.

In one embodiment, a method for providing guided behavioral therapy and skills training comprises: obtaining patient profile and pre-assessment data; generating patient condition data based on the patient profile and pre-assessment data; processing the patient profile and pre-assessment data and the patient condition data to generate a personalized intervention regimen for the patient wherein the personalized intervention regimen for the patient defines one or more interactive therapy modules to be administered to the patient; administering the one or more interactive therapy modules to the patient according to the personalized intervention regimen generated for the patient; monitoring the patient's interactions with the content of the one or more interactive therapy modules to generate patient interaction data representing the patient's interactions with the content of the one or more interactive therapy modules; at least partly based on the patient interaction data, dynamically controlling patient progression through the interactive therapy modules; at least partly based on the patient interaction data, dynamically updating the patient's personalized intervention regimen; and administering one or more interactive therapy modules to the patient according to the updated personalized intervention regimen generated for the patient.

In one embodiment, a system comprises one or more processors and one or more physical memories, the one or more physical memories having stored therein data representing instructions which when processed by the one or more processors perform the above described methods/processes.

The systems and methods disclosed herein allow behavioral therapy to be administered to patients suffering from gastrointestinal (GI) and/or inflammatory health conditions in a convenient and flexible, yet structured fashion, via a prescription digital therapeutics (PDT) system. The invention(s) can employ non-traditional systems and methods for providing interventions to patients exhibiting symptoms associated with one or more health conditions. In some embodiments, the invention(s) can deliver psychological-based interventions, such as behavioral therapy-based interventions and other interventions (described in more detail above) to users/patients, by way of a platform having components implemented in a mobile device environment and/or other computer or internet-based architecture. Thus, in various embodiments, the invention(s) use components of the platform to process large amounts of user data, remotely deliver personalized interventions, and remotely monitor user interactions with such interventions in near real-time in a manner that cannot be practically implemented by the human mind.

Consequently, the embodiments disclosed herein are not an abstract idea, and are well-suited to a wide variety of practical applications. Further, many of the embodiments disclosed herein require processing and analysis of billions of data points and combinations of data points, and thus, the technical solution disclosed herein cannot be implemented solely by mental steps or pen and paper, is not an abstract idea, and is, in fact, directed to providing technical solutions to long-standing technical problems associated with effectively, efficiently, and remotely treating gastrointestinal and inflammatory health conditions using prescription digital therapeutics in order to ensure that patients receive adequate care, support, and treatment.

Additionally, the disclosed method and system for effectively, efficiently, and remotely treating gastrointestinal and inflammatory health conditions using prescription digital therapeutics requires specific processes that utilize components of the platform disclosed herein to process user data, deliver interventions, and monitor user interactions with such interventions, and as such, does not encompass, embody, or preclude other forms of innovation in the area of healthcare technologies. Further, the disclosed embodiments of systems and methods for dynamically, efficiently, and remotely treating gastrointestinal and inflammatory health conditions using prescription digital therapeutics are not abstract ideas for at least several reasons.

First, effectively, efficiently, and remotely treating gastrointestinal and inflammatory health conditions using prescription digital therapeutics is not an abstract idea because it is not merely an idea in and of itself. For example, the process cannot be performed mentally or using pen and paper, as it is not possible for the human mind to utilize components of the platform disclosed herein, to process large amounts of user data, remotely deliver personalized interventions, and remotely monitor user interactions with such interventions in near real-time, even with pen and paper to assist the human mind and even with unlimited time.

Second, effectively, efficiently, and remotely treating gastrointestinal and inflammatory health conditions using prescription digital therapeutics is not a fundamental economic practice (e.g., is not merely creating a contractual relationship, hedging, mitigating a settlement risk, etc.).

Third, effectively, efficiently, and remotely treating gastrointestinal and inflammatory health conditions using prescription digital therapeutics is not merely a method of organizing human activity (e.g., managing a game of bingo). Rather, in the disclosed embodiments, the method and system for effectively, efficiently, and remotely treating gastrointestinal and inflammatory health conditions using prescription digital therapeutics provides a tool that significantly improves the fields of medical and mental health care for patients suffering from a wide variety of diseases and/or conditions. Through the disclosed embodiments, health practitioners are provided with a tool to help them generate personalized and adaptive intervention regimens for use in treating gastrointestinal and inflammatory health conditions using prescription digital therapeutics, which ensures that patients are provided with personalized and effective assistance, treatment, and care. As such, the method and system disclosed herein is not an abstract idea, and also serves to integrate the ideas disclosed herein into practical applications of those ideas.

Fourth, although mathematics may be used to implement the embodiments disclosed herein, the systems and methods disclosed and claimed herein are not abstract ideas because the disclosed systems and methods are not simply a mathematical relationship/formula.

It should be noted that the language used in the specification has been principally selected for readability, clarity, and instructional purposes, and may not have been selected to delineate or circumscribe the inventive subject matter. Accordingly, the disclosure of the present invention is intended to be illustrative, but not limiting, of the scope of the invention, which is set forth in the claims below.

The present invention has been described in particular detail with respect to specific possible embodiments. Those of skill in the art will appreciate that the invention may be practiced in other embodiments. For example, the nomenclature used for components, capitalization of component designations and terms, the attributes, data structures, or any other programming or structural aspect is not significant, mandatory, or limiting, and the mechanisms that implement the invention or its features can have various different names, formats, or protocols. Further, the system or functionality of the invention may be implemented via various combinations of software and hardware, as described, or entirely in hardware elements. Also, particular divisions of functionality between the various components described herein are merely exemplary, and not mandatory or significant. Consequently, functions performed by a single component may, in other embodiments, be performed by multiple components, and functions performed by multiple components may, in other embodiments, be performed by a single component.

In the discussion above, certain aspects of one embodiment include process steps and/or operations and/or instructions described herein for illustrative purposes in a particular order and/or grouping. However, the particular order and/or grouping shown and discussed herein are illustrative only and not limiting. Those of ordinary skill in the art will recognize that other orders and/or grouping of the process steps and/or operations and/or instructions are possible and, in some embodiments, one or more of the process steps and/or operations and/or instructions discussed above can be combined and/or deleted. In addition, portions of one or more of the process steps and/or operations and/or instructions can be re-grouped as portions of one or more other of the process steps and/or operations and/or instructions discussed herein. Consequently, the particular order and/or grouping of the process steps and/or operations and/or instructions discussed herein do not limit the scope of the invention as claimed below.

As discussed in more detail above, using the above embodiments, with little or no modification and/or input, there is considerable flexibility, adaptability, and opportunity for customization to meet the specific needs of various parties under numerous circumstances.

Some portions of the above description present the features of the present invention in terms of algorithms and symbolic representations of operations, or algorithm-like representations, of operations on information/data. These algorithmic or algorithm-like descriptions and representations are the means used by those of skill in the art to most effectively and efficiently convey the substance of their work to others of skill in the art. These operations, while described functionally or logically, are understood to be implemented by computer programs or computing systems. Furthermore, it has also proven convenient at times to refer to these arrangements of operations as steps or modules or by functional names, without loss of generality.

Unless specifically stated otherwise, as would be apparent from the above discussion, it is appreciated that throughout the above description, discussions utilizing terms such as, but not limited to, “activating”, “accessing”, “adding”, “aggregating”, “alerting”, “applying”, “analyzing”, “associating”, “calculating”, “capturing”, “categorizing”, “classifying”, “comparing”, “creating”, “defining”, “detecting”, “determining”, “distributing”, “eliminating”, “encrypting”, “extracting”, “filtering”, “forwarding”, “generating”, “identifying”, “implementing”, “informing”, “monitoring”, “obtaining”, “posting”, “processing”, “providing”, “receiving”, “requesting”, “saving”, “sending”, “storing”, “substituting”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

The present invention also relates to an apparatus or system for performing the operations described herein. This apparatus or system may be specifically constructed for the required purposes, or the apparatus or system can comprise a system selectively activated or configured/reconfigured by a computer program stored on a non-transitory computer readable medium for carrying out instructions using a processor to execute a process, as discussed or illustrated herein that can be accessed by a computing system or other device.

Those of ordinary skill in the art will readily recognize that the algorithms and operations presented herein are not inherently related to any particular computing system, computer architecture, computer or industry standard, or any other specific apparatus. Various systems may also be used with programs in accordance with the teaching herein, or it may prove more convenient/efficient to construct more specialized apparatuses to perform the required operations described herein. The required structure for a variety of these systems will be apparent to those of ordinary skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the invention as contemplated by the inventors at the time of filing.

The present invention is well suited to a wide variety of computer network systems operating over numerous topologies. Within this field, the configuration and management of large networks comprise storage devices and computers that are communicatively coupled to similar or dissimilar computers and storage devices over a private network, a LAN, a WAN, a private network, or a public network, such as the Internet.

It should also be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below.

In addition, the operations shown in the figures, or as discussed herein, are identified using a particular nomenclature for ease of description and understanding, but other nomenclature is often used in the art to identify equivalent operations.

Therefore, numerous variations, whether explicitly provided for by the specification or implied by the specification or not, may be implemented by one of skill in the art in view of this disclosure.

What is claimed is:

1. A computing system implemented method comprising:
providing a patient with a user interface to a therapeutics system;
performing, by the therapeutics system, a pre-assessment of a patient exhibiting one or more health condition symptoms;
generating, by the therapeutics system, patient profile and pre-assessment data based on the results of the patient pre-assessment;
processing, by the therapeutics system, the patient profile and pre-assessment data to generate patient condition data;
processing, by the therapeutics system, the patient profile and pre-assessment data and the patient condition data to generate a personalized intervention regimen for the patient wherein the personalized intervention regimen for the patient defines one or more interactive therapy modules to be administered to the patient;
administering the one or more interactive therapy modules to the patient through the user interface of the therapeutics system according to the personalized intervention regimen generated for the patient;
monitoring the patient's interactions with the content of the one or more interactive therapy modules to generate patient interaction data representing the patient's interactions with the content of the one or more interactive therapy modules;
at least partly based on the patient interaction data, dynamically controlling patient progression through the one or more interactive therapy modules;
at least partly based on the patient interaction data, dynamically updating the patient's personalized intervention regimen; and
administering one or more interactive therapy modules to the patient through the user interface of the therapeutics system according to the updated personalized intervention regimen generated for the patient.

2. The computing system implemented method of claim 1, wherein the therapeutics system is a prescription digital therapeutics (PDT) system.

3. The computing system implemented method of claim 1 wherein the patient health condition symptoms are associated with one or more of:
a gastrointestinal (GI) health condition; and
an inflammatory health condition.

4. The computing system implemented method of claim 1, wherein performing a pre-assessment of a patient includes utilizing surveying tools derived from one or more patient reported outcome instruments selected from the group of patient reported outcome instruments consisting of:
a Functional Bowel Disorder Severity Index (FBDSI) instrument;
a Gastrointestinal Symptom Rating Scale-IBS (GSRS-IBS) instrument;
an IBS-Adequate Relief (IBS-AR) instrument;
an IBS Global Assessment of Improvement Scale (IBS-GAI) instrument;
an IBS Symptom Severity Score (IBS-SSS) instrument;
an IBS Quality of Life (IBS-QOL) instrument;
a Patient Reported Outcome Measurement Information System (PROMIS) instrument;
a Visual Analogue Scale for Irritable Bowel Syndrome (VAS-IBS);
a work and social adjustment scale (WSAS)-derived instrument;
a clinical disease activity measurement instrument;
a PHQ-9 patient health questionnaire;
a GAD-7 anxiety disorder questionnaire; and
a pain assessment questionnaire.

5. The computing system implemented method of claim 1, wherein performing a pre-assessment of a patient includes utilizing non-survey data obtained from one or more devices selected from the group of devices consisting of:
electronic health record-associated devices;
torso-coupled devices;
wearable devices;
ingestible devices;
implanted devices;
smart-pill devices;
smart toilet devices;
location monitoring devices; and
social networking tracking devices.

6. The computing system implemented method of claim 1 wherein generating the patient condition data includes of one or more of:
identifying the patient condition as a gastrointestinal (GI) health condition;
identifying a subtype of the gastrointestinal (GI) health condition;
identifying a severity of the gastrointestinal (GI) health condition;
identifying the patient condition as an inflammatory health condition;
identifying a subtype of the inflammatory health condition; and
identifying a severity of the inflammatory health condition.

7. The computing system implemented method of claim 1, wherein the one or more interactive therapy modules administered to the patient according to the personalized intervention regimen for the patient are part of a guided behavioral therapy treatment, further wherein the guided behavioral therapy treatment is administered remotely through a user interface of the therapeutics system.

8. The computing system implemented method of claim 7, wherein the one or more interactive therapy modules of the guided behavioral therapy treatment include components of therapies selected from the group of therapies consisting of:
psychotherapy;
cognitive behavioral therapy (CBT);
acceptance commitment therapy (ACT);
dialectical behavioral therapy (DBT);
exposure therapy;
mindfulness-based cognitive therapy (MCBT);
hypnotherapy;
experiential therapy; and
psychodynamic therapy.

9. The computing system implemented method of claim 8, further wherein the components of the one or more interactive therapy modules include one or more of:
relaxation techniques training;
behavioral change and avoidance training;
problem solving and coping training;
pain management techniques training;
cognitive restructuring and flexibility training;
social problem solving and communication training; and
relapse prevention and skills maintenance training.

10. The computing system implemented method of claim 1 wherein generating a personalized intervention regimen includes one or more of:
defining a plurality of interactive therapy modules;
processing, through the therapeutics system, the patient profile and pre-assessment data and the patient condition data to identify one or more of the plurality of interactive therapy modules to be administered to the patient;
defining a plurality of therapeutic protocols to be utilized in administration of one or more interactive therapy modules;
processing, through the therapeutics system, the patient profile and pre-assessment data and the patient condition data to identify one or more of the plurality of therapeutic protocols to utilize while administering the identified one or more interactive therapy modules to the patient; and
processing, by the therapeutics system, the identified one or more interactive therapy modules and the identified one or more therapeutic protocols to generate a personalized intervention regimen for the patient.

11. The computing system implemented method of claim 1 wherein monitoring the patient's interactions with the content of the interactive therapy modules includes obtaining one or more of:
patient physiological health data;
patient psychological health data;
patient condition data;
patient symptoms data;
patient medications data;
patient medication adherence data;
patient progress report data;
patient system usage data;
patient device usage data;
patient social networking behavior data;
patient voice data;
patient textual data;
patient activity data;
patient location data;
patient motion data; and
patient biometric data.

12. The computing system implemented method of claim 1 wherein the patient's interactions with the content of the interactive therapy modules are monitored in near-real time, contemporaneously with administration of the interactive therapy modules.

13. The computing system implemented method of claim 1 wherein dynamically controlling patient progression through the interactive therapy modules includes:
defining criteria for continued progression through the interactive therapy modules;
processing patient interaction data representing the patient's interactions with the content of the interactive therapy modules to determine whether the criteria for continued progression should be dynamically adjusted for the patient;
processing patient interaction data representing the patient's interactions with the content of the interactive therapy modules to determine whether the patient has met the criteria for continued progression through the interactive therapy modules;
upon a determination that the patient has not met the criteria for continued progression through the interactive therapy modules, implementing one of:
a soft-gate, wherein the soft-gate introduces friction to impede the patient's progress from one therapy module to a subsequent therapy module; or
a hard-gate, wherein the hard-gate prevents the patient from progressing from one therapy module to a subsequent therapy module until the patient has met the defined criteria.

14. The computing system implemented method of claim 13, wherein the criteria for continued progression through the interactive therapy modules are based on one or more of:
an elapsed time from completion of one therapy module to the next therapy module;
an aggregate usage time of the therapeutics system;
the number of therapy modules completed within a specific period of time;
an evaluation of the patient's understanding of the content provided in the therapy modules;
an evaluation of the patient's psychological state while the therapy modules are being administered; and
an evaluation of the patient's physiological state while the therapy modules are being administered.

15. The computing system implemented method of claim 13, wherein a machine learning module is utilized to evaluate a patient's usage habits with respect to criteria for continued progression through the interactive therapy modules, further wherein the machine learning module determines whether to implement a soft-gate or a hard-gate.

16. The computing system implemented method of claim 13 wherein patient progression through the interactive therapy modules is dynamically controlled in near real-time.

17. The computing system implemented method of claim 1 wherein dynamically updating the patient's personalized intervention regimen includes one or more of:
adjusting the order of administration of the interactive therapy modules;
adjusting the frequency of administration of the interactive therapy modules;
adjusting the mode of administration of the interactive therapy modules;
adjusting the content of the interactive therapy modules;
adjusting the content size of the interactive therapy modules;
adjusting the presentation of the interactive therapy modules;
adjusting the layout of the interactive therapy modules;
updating the patient's electronic health records;
updating the patient's personal health records;
updating the patient's open medical records; and
increasing personalization of the intervention regimen.

18. The computing system implemented method of claim 1 wherein the patient's personalized intervention regimen is updated in near-real time.

19. A system comprising:
one or more processors; and
one or more physical memories, the one or more physical memories having stored therein data representing instructions which when processed by the one or more processors perform a process, the process comprising:
providing a patient with a user interface to a therapeutics system;
performing, by the therapeutics system, a pre-assessment of a patient exhibiting one or more health condition symptoms;
generating, by the therapeutics system, patient profile and pre-assessment data based on the results of the patient pre-assessment;
processing, by the therapeutics system, the patient profile and pre-assessment data to generate patient condition data;
processing, by the therapeutics system, the patient profile and pre-assessment data and the patient condition data to generate a personalized intervention regimen for the patient wherein the personalized intervention regimen for the patient defines one or more interactive therapy modules to be administered to the patient;
administering the one or more interactive therapy modules to the patient through the user interface of therapeutics system according to the personalized intervention regimen for the patient;
monitoring the patient's interactions with the content of the one or more interactive therapy modules to generate patient interaction data representing the patient's interactions with the content of the one or more interactive therapy modules;
at least partly based on the patient interaction data, dynamically controlling patient progression through the interactive modules;
at least partly based on the patient interaction data, dynamically updating the patient's personalized intervention regimen; and
administering one or more interactive therapy modules to the patient through a user interface of the therapeutics system according to the updated personalized intervention regimen for the patient.

20. The system of claim 19, wherein the therapeutics system is a prescription digital therapeutics (PDT) system.

21. The system of claim 19 wherein the patient health condition symptoms are associated with one or more of:
a gastrointestinal (GI) health condition; and
an inflammatory health condition.

22. The system of claim 19, wherein performing a pre-assessment of a patient includes utilizing surveying tools derived from one or more patient reported outcome instruments selected from the group of patient reported outcome instruments consisting of:
a Functional Bowel Disorder Severity Index (FBDSI) instrument;
a Gastrointestinal Symptom Rating Scale-IBS (GSRS-IBS) instrument;
an IBS-Adequate Relief (IBS-AR) instrument;
an IBS Global Assessment of Improvement Scale (IBS-GAI) instrument;
an IBS Symptom Severity Score (IBS-SSS) instrument;
an IBS Quality of Life (IBS-QOL) instrument;
a Patient Reported Outcome Measurement Information System (PROMIS) instrument;
a Visual Analogue Scale for Irritable Bowel Syndrome (VAS-IBS);
a work and social adjustment scale (WSAS)-derived instrument;
a clinical disease activity measurement instrument;
a PHQ-9 patient health questionnaire;
a GAD-7 anxiety disorder questionnaire; and
a pain assessment questionnaire.

23. The system of claim 19, wherein performing a pre-assessment of a patient includes utilizing non-survey data obtained from one or more devices selected from the group of devices consisting of:
electronic health record-associated devices;
torso-coupled devices;
wearable devices;
ingestible devices;
implanted devices;
smart-pill devices;
smart toilet devices;
location monitoring devices; and
social networking tracking devices.

24. The system of claim 19 wherein generating the patient condition data includes of one or more of:
identifying the patient condition as a gastrointestinal (GI) health condition;
identifying a subtype of the gastrointestinal (GI) health condition;
identifying a severity of the gastrointestinal (GI) health condition;
identifying the patient condition as an inflammatory health condition;
identifying a subtype of the inflammatory health condition; and
identifying a severity of the inflammatory health condition.

25. The system of claim 19, wherein the one or more interactive therapy modules administered to the patient according to the personalized intervention regimen for the patient are part of a guided behavioral therapy treatment, further wherein the guided behavioral therapy treatment is administered remotely through a user interface of the therapeutics system.

26. The system of claim 25, wherein the one or more interactive therapy modules of the guided behavioral therapy treatment include components of therapies selected from the group of therapies consisting of:
psychotherapy;
cognitive behavioral therapy (CBT);
acceptance commitment therapy (ACT);
dialectical behavioral therapy (DBT);
exposure therapy;
mindfulness-based cognitive therapy (MCBT);
hypnotherapy;
experiential therapy; and
psychodynamic therapy.

27. The system of claim 26, further wherein the components of the one or more interactive therapy modules include one or more of:
relaxation techniques training;
behavioral change and avoidance training;
problem solving and coping training;
pain management techniques training;
cognitive restructuring and flexibility training;
social problem solving and communication training; and
relapse prevention and skills maintenance training.

28. The system of claim 19 wherein generating a personalized intervention regimen includes one or more of:
defining a plurality of interactive therapy modules;
processing, through the therapeutics system, the patient profile and pre-assessment data and the patient condition data to identify one or more of the plurality of interactive therapy modules to be administered to the patient;
defining a plurality of therapeutic protocols to be utilized in administration of one or more interactive therapy modules;
processing, through the therapeutics system, the patient profile and pre-assessment data and the patient condition data to identify one or more of the plurality of therapeutic protocols to utilize while administering the identified one or more interactive therapy modules to the patient; and
processing, by the therapeutics system, the identified one or more interactive therapy modules and the identified one or more therapeutic protocols to generate a personalized intervention regimen for the patient.

29. The system of claim 19 wherein monitoring the patient's interactions with the content of the interactive therapy modules includes obtaining one or more of:
patient physiological health data;
patient psychological health data;
patient condition data;
patient symptoms data;
patient medications data;
patient medication adherence data;
patient progress report data;
patient system usage data;
patient device usage data;
patient social networking behavior data;
patient voice data;
patient textual data;
patient activity data;
patient location data;
patient motion data; and
patient biometric data.

30. The system of claim 19 wherein the patient's interactions with the content of the interactive therapy modules are monitored in near-real time, contemporaneously with administration of the interactive therapy modules.

31. The system of claim 19 wherein dynamically controlling patient progression through the interactive therapy modules includes:
defining criteria for continued progression through the interactive therapy modules;
processing patient interaction data representing the patient's interactions with the content of the interactive therapy modules to determine whether the criteria for continued progression should be dynamically adjusted for the patient;
processing patient interaction data representing the patient's interactions with the content of the interactive therapy modules to determine whether the patient has met the criteria for continued progression through the interactive therapy modules;
upon a determination that the patient has not met the criteria for continued progression through the interactive therapy modules, implementing one of:
a soft-gate, wherein the soft-gate introduces friction to impede the patient's progress from one therapy module to a subsequent therapy module; or
a hard-gate, wherein the hard-gate prevents the patient from progressing from one therapy module to a subsequent therapy module until the patient has met the defined criteria.

32. The system of claim 31, wherein the criteria for continued progression through the interactive therapy modules are based on one or more of:
an elapsed time from completion of one therapy module to the next therapy module;
an aggregate usage time of the therapeutics system;
the number of therapy modules completed within a specific period of time;
an evaluation of the patient's understanding of the content provided in the therapy modules;
an evaluation of the patient's psychological state while the therapy modules are being administered; and
an evaluation of the patient's physiological state while the therapy modules are being administered.

33. The system of claim 31, wherein a machine learning module is utilized to evaluate a patient's usage habits with respect to criteria for continued progression through the interactive therapy modules, further wherein the machine learning module determines whether to implement a soft-gate or a hard-gate.

34. The system of claim 19 wherein patient progression through the interactive therapy modules is dynamically controlled in near real-time.

35. The system of claim 19 wherein dynamically updating the patient's personalized intervention regimen includes one or more of:
adjusting the order of administration of the interactive therapy modules;
adjusting the frequency of administration of the interactive therapy modules;
adjusting the mode of administration of the interactive therapy modules;
adjusting the content of the interactive therapy modules;
adjusting the content size of the interactive therapy modules;
adjusting the presentation of the interactive therapy modules;
adjusting the layout of the interactive therapy modules;
updating the patient's electronic health records;
updating the patient's personal health records;
updating the patient's open medical records; and
increasing personalization of the intervention regimen.

36. The system of claim 19 wherein the patient's personalized intervention regimen is updated in near-real time.

37. A method comprising:
obtaining patient profile and pre-assessment data;
generating patient condition data based on the patient profile and pre-assessment data;
processing the patient profile and pre-assessment data and the patient condition data to generate a personalized intervention regimen for the patient wherein the personalized intervention regimen for the patient defines one or more interactive therapy modules to be administered to the patient;
administering the one or more interactive therapy modules to the patient according to the personalized intervention regimen generated for the patient;
monitoring the patient's interactions with the content of the one or more interactive therapy modules to generate patient interaction data representing the patient's interactions with the content of the one or more interactive therapy modules;
at least partly based on the patient interaction data, dynamically controlling patient progression through the interactive therapy modules;
at least partly based on the patient interaction data, dynamically updating the patient's personalized intervention regimen; and
administering one or more interactive therapy modules to the patient according to the updated personalized intervention regimen generated for the patient.
